(19) United States
(12) Patent Application Publication
(10) Pub. No.: US 2018/0036375 A1
(43) Pub. Date: Feb. 8, 2018

(54) Spatiotemporal Delivery Vehicle and Related Methods

(71) Applicant: University of Pittsburgh - Of the Commonwealth System of Higher Eduction, Pittsburgh, PA (US)

(72) Inventors: Hassan K. Awada, Pittsburgh, PA (US); Yadong Wang, Bradford Woods, PA (US)

(21) Appl. No.: 15/555,277

(22) PCT Filed: Mar. 4, 2016
(86) PCT No.: PCT/US2016/020935

§ 371 Date: Sep. 1, 2017

Related U.S. Application Data
(60) Provisional application No. 62/129,298, filed on Mar. 6, 2015.

(51) Int. Cl. A61K 38/18; A61K 47/42; A61K 47/36; A61K 38/19; A61K 38/55

(52) U.S. Cl. CPC A61K 38/18; A61K 38/19; A61K 38/55; A61K 47/36; A61K 47/42; A61K 9/0019

(57) ABSTRACT

A drug delivery composition comprising a coacervate embedded in a hydrogel is provided. Methods of making and using the drug delivery composition also are provided, including a treatment composition and method of treating myocardial infarction.

### CROSS REFERENCE TO RELATED APPLICATIONS

This application claims the benefit of U.S. Provisional Patent Application No. 62/129,298, filed Mar. 6, 2015, which is incorporated herein by reference in its entirety.

### STATEMENT REGARDING FEDERAL FUNDING

This invention was made with government support under Grant No. EB003392 awarded by the National Institutes of Health, and Grant No. DMR1005766 awarded by the National Science Foundation. The government has certain rights in the invention.

### BACKGROUND

### 1. Field of the Invention

Provided herein is a composition useful for spatiotemporal control of drug delivery. Also provided herein are methods of making and using the drug delivery composition.

### 2. Description of Related Art

Approximately 720,000 Americans experience a heart attack each year, or one American every 44 seconds. Ischemic heart disease is a leading cause of morbidity and mortality in the United States. Approximately, 15% of the people who experience a heart attack (myocardial infarction) in a given year will die of it. Myocardial infarction leads to the prolonged starvation of a portion of the heart muscle (infarct zone) of blood flow, oxygen, and nutrients due to an occlusion in one of the two coronary arteries. This leads to defects in the contractile function of cardiomyocytes and alterations in extracellular matrix (ECM) and the left ventricle (LV) geometry. Briefly, the ischemic cardiac tissue starts experiencing necrosis and cell apoptosis, perivascular fibrosis, and fibrillar collagen deposition around myocytes. As a result of all these pathological changes, a scar tissue forms and a pathological remodeling of the ventricle starts, eventually leading to congestive heart failure.

Current treatments for MI patients, such as reperfusion, β-blockers, and ACE inhibitors, do not suffice. They are able to delay further damage to the heart, but have not been successful at inducing significant cardiac repair and regeneration. Therefore, new more comprehensive therapies that can reduce the damage of infarction, prevent or reverse the multiple pathologies developed by MI, regenerate the myocardium, and restore cardiac function are urgently needed.

Therapeutic angiogenesis aims to restore blood flow to the affected ischemic heart muscles by new blood vessel formation from existing vasculature. Revascularization by pro-angiogenic therapies has thus far failed to provide satisfactory outcomes in clinical trials. Bolus injections of single growth factors led to limited efficacy because of loss of bioactivity, missing critical signals in the cascade of events that lead to stable angiogenesis, among others. An effective angiogenesis-based therapy is needed, and can be developed when a comprehensive understanding of angiogenic mechanisms becomes available. Repair and regeneration strategies should focus on utilizing the growth factors that play vital roles in the process of angiogenesis, as well as the need to administer them spatiotemporally and in bioactive conformations.

Many studies have shown that growth factors such as fibroblast growth factor-2 (FGF-2), vascular endothelial growth factor (VEGF), angiopoietin-2 (Ang-2) are key factors in triggering angiogenesis, but these factors alone may result in leaky and immature blood vessels that are susceptible to early regression. Other growth factors such as platelet-derived growth factor (PDGF) and angiopoietin-1 (Ang-1) help stabilize neovessels. Among potential angiogenic candidates, VEGF and PDGF are promising due to their potency, specificity, and cardioprotective roles. VEGF, an endothelial-specific factor, triggers the process through endothelial cell (EC) sprouting, proliferation, migration, and lumen formation, and is thus primarily needed in the first few days of angiogenesis. After lumenal formation, mural cells are recruited by PDGF to cover the neovessels and provide stabilization; therefore PDGF is required at a later stage of angiogenesis to prevent vessel regression or the formation of aberrant and leaky vessels. It has been shown that early-stage angiogenic factors can have antagonistic effects on late-stage factors and vice versa, when present simultaneously. Therefore, it appears imperative to sequentially administer these two growth factors to imitate their physiological presence during angiogenesis. Effective delivery compositions are needed in order to administer the two growth factors. More generally, effective delivery platforms are needed to temporally and spatially control drug delivery.

### SUMMARY

Provided herein is a controlled delivery system made of a combination of fibrin gel and a coacervate system. Fibrin gel is made by the polymerization of fibrinogen into fibrin, mediated by thrombin. The coacervate is formed by the mixing of an active agent, such as a drug or protein with heparin and a custom-made polycation (e.g., PEAD). Complex coacervates are formed by mixing oppositely charged polyelectrolytes resulting in spherical droplets of organic molecules held together non-covalently and apart from the surrounding liquid. This combinatorial approach provides a higher level of control over the release of drugs from a delivery system, and is shown herein to be effective at controlled spatial and temporal delivery of biologics. Embedding a drug in fibrin gel leads to a quick release of this drug over few days, while embedding a drug into the coacervate, and distributing it in the same fibrin gel leads to a relatively longer sustained release over many weeks. This sequential release of drugs is important in processes where the temporal component is a factor. For example, in angiogenesis, there are proteins needed early on, to trigger the formation of new blood vessels, and there are other proteins needed at late stage to stabilize the newly formed vessels.

The delivery system and methods described herein provide the capability of sequential controlled release of drugs, or more specifically, proteins. This existing coacervate system can sustain the release of drugs for weeks. The addition of fibrin gel and embedding proteins in the gel, and not in the coacervate, leads to a temporal separation of drugs, providing a new level of control over the release of drugs not available by either coacervate or fibrin gel alone.

### BRIEF DESCRIPTION OF THE DRAWINGS

FIG. 1. Chemical structure of poly(ethylene argininylaspartate diglyceride), PEAD.

FIG. 2. Binding ability of PEAD to heparin (FIG. 10A) zeta potential measurement (FIG. 10B) DMB binding assay

FIG. 3. Scanning electron microscopic images of PEAD/heparin complex (FIG. 11A) low magnification (2,000×) (FIG. 11B) high magnification (10,000×).

FIG. 4. Release profiles of PEAD/heparin-complexed growth factor (FIG. 12A) FGF-2 (FIG. 12B) NGF.

FIG. 5. HAECs proliferation promoted by FGF-2: (1) control, (2) EC culture supplement, (3) bolus FGF-2 and (4) PEAD/heparin-complexed FGF-2.

FIG. 6. PC-12 differentiation stimulated by NGF: (1) control, (2) bolus NGF, (3) heparin-stabilized NGF and (4) PEAD/heparin-complexed NGF (FIG. 14A) quantification of neurite lengths (FIG. 14B) phase contract images after 7 days.

FIGS. 7(A-F). (FIG. 7(A)) Chemical structure of poly(ethylene argininylaspartate diglyceride) (PEAD). The backbone of PEAD composed of aspartic acid and ethylene glycol diglyceride is linked together by ester bonds. The conjugation of arginine renders the polymer two positive charges, ammonium and guanidinium moieties, per repeating unit at the physiological condition. (FIG. 7(B)) Heparin has high solubility in the aqueous solution. Once addition of PEAD, the solution becomes turbid due to neutralization of negative charges. [PEAD:Heparin] complex forms coacervate and precipitates to the bottom after 24 h incubation. (FIG. 7(C)) SEM micrograph revealed the fibrous and globular features of [PEAD:Heparin] complex. Scale bar, 1 μm. (FIG. 7(D)) Schematic representation of the interaction between the heparin-binding growth factors and [PEAD:Heparin] complex. The Coulombic attraction between PEAD (yellow) and heparin (blue) is able to incorporate heparin-binding growth factors (red) into the complex. (FIG. 7(E)) The loading efficiency examined by western blot suggested that [PEAD:Heparin] can incorporate the majority of FGF-2 in the solution. Continuing increasing the ionic strength of the solution to 5 folds of the normal saline condition disrupted the interaction between PEAD, heparin and FGF-2. Therefore, FGF-2 was not precipitated after centrifugation. (FIG. 7(F)) In vitro tube formation of HUVECs in the 3D fibrin gel. HUVECs mixed with bolus FGF-2 or the delivery matrix were encapsulated in the fibrin gel. After incubation for 3 days, the image of the delivery matrix group revealed an evident tube network connected by differentiated cells. On the contrary, bolus FGF-2 only induced sparse cells scattering in the gel. Scale bar: 100 μm.

FIG. 8. Release of VEGF and PDGF from the coacervate. The coacervate was formed by mixing 100 ng of each GF together, then with heparin, then with PEAD polycation. Release of each GF was detected by ELISA. The coacervate released 57% of PDGF compared to 35% of VEGF by 3 weeks. Data are presented as means±SD (n=3).

FIG. 9. Sequential delivery of VEGF and PDGF using a fibrin gel-coacervate system. (A) The delivery system was comprised of a fibrin gel embedding free VEGF and PDGF-loaded coacervate droplets. The coacervate was formed through electrostatic interactions by combining PDGF with heparin then with PEAD polycation (B) The delivery system described achieved sequential quick release of VEGF followed by a sustained release of PDGF. Data are presented as means±SD (n=3 per group).

FIG. 10. PDGF coacervate promotes smooth muscle cell (SMC) chemotaxis and proliferation. (A) After 12 h, images show more migrated SMC through the cell culture insert membrane towards PDGF coacervate compared to other groups. (B) Although free PDGF significantly induced migration compared to control, it was less than PDGF coacervate which significantly enhanced migration compared to all other groups. (C) After 48 h, free PDGF induced significantly more SMC proliferation than controls, while PDGF coacervate induced significantly more proliferation than all groups. Proliferation values were normalized to basal media average. Data are presented as means±SD (n=3 per group). **P<0.01. Scale bar=250 μm.

FIG. 11. Sequential delivery of VEGF and PDGF promotes endothelial cell proliferation and vessel sprouting. (A) After 48 h, free GFs (VEGF+PDGF) induced significantly more endothelial proliferation than basal media, while sequential delivery of VEGF and PDGF induced significantly more proliferation than all groups. Proliferation values were normalized to basal media average. (B) After 6 days, rat aortic ring assay shows that free GFs induced significantly larger microvasculature sprouting area than basal media. Sequential delivery induced significantly larger sprouting areas compared to all groups. (C) Representative images show microvasculature formation around rat aortic rings, with more sprouting observed in the sequential delivery group. Data are presented as means±SD (n=3 per group). *P<0.05, **P<0.01. Scale bar=500 μm.

FIG. 12. Sequential delivery of VEGF and PDGF improves cardiac function after MI. (A) End-systolic area (ESA) and (B) End-diastolic area (EDA) showed no statistical difference between groups at all time points suggesting no effect on ventricular dilation. (C) % Fractional area change (FAC) reflected a significantly improved cardiac contractility at 2 wks and maintained at 4 wks in the sequential delivery group compared to all groups. In comparison, sequential delivery group displayed a 68% improvement over saline and 60% over free GFs at 2 wks. Data are presented as means±SD (n=7 per group). **P<0.01.

FIG. 13. Sequential delivery of VEGF and PDGF improves ventricular wall thickness and reduces fibrosis 4 wks after MI. (A) H&E staining showed ventricular wall thinning with damaged cardiac muscle surrounded by scar tissue in saline, empty vehicle, and free GFs groups. However, these damages were apparently alleviated in the sequential delivery group. Quantitative analysis showed (B) significantly increased ventricular wall thickness and (C) significantly reduced collagen deposition in the sequential delivery group compared to all groups. (D) Picosirius red staining images show the vast collagen deposition areas along the LV wall and infarct zone in saline, empty vehicle, and free GFs groups. Collagen deposition was reduced in the sequential delivery group indicating less fibrotic tissue and scar formation. Data are presented as means±SD (n=5-6 per group). *P<0.05, **P<0.01. Scale bar=1000 μm.

FIG. 14. Sequential delivery of VEGF and PDGF improves angiogenesis 4 wks after MI. (A) Representative images show co-staining of VWF (red) and α-SMA (green) that reflect the level of neovessel formation, their functionality and maturity, with noticeable improved angiogenesis in the sequential delivery group. (B) Saline and empty vehicle groups showed little angiogenesis with few VWF-positive vessels. While free GFs induced significantly more VWF-positive vessels than controls, sequential delivery induced significantly more than all groups. (C) Sequential delivery induced significantly more α-SMA-positive vessels than all groups. Data are presented as means±SD (n=4-5 per group). *P<0.05, **P<0.01. Scale bar=200 μm.

FIG. 15. Sequential delivery of VEGF and PDGF improves cardiac muscle viability and reduces inflammation 4 wks after MI. (A) Cardiac troponin I (cTnI) staining (green) showed few viable cardiomyocytes in saline, empty vehicle, and free GFs groups, while sequential delivery group showed a larger area of viable cardiac muscle in the infarct zone. Scale bar=500 μm. (B) Quantitative analysis revealed that the sequential delivery group showed a significantly larger cTnI-positive area fraction in the infarct region compared to all groups. (C) Staining of inflammatory marker CD68 showed large numbers of CD68-positive cells in saline and empty vehicle groups, while significantly less cells were found in free GFs group and even less in sequential delivery group, with no significant difference between them. (D) Representative images of CD68 staining show less positive (red) cells in free GFs and sequential delivery groups. Scale bar=250 μm. Data are presented as means±SD (n=4-5 per group). *P<0.05.

FIG. 16. Protein roles in cardiac repair. The four proteins FGF-2, SDF-1α, IL-10, and TIMP-3 have relatively distinct but complementary cardiac functions. FGF-2 promotes angiogenesis by endothelial sprouting and pericyte recruitment and also cardiomyocyte survival. SDF1-α has the critical role of recruiting cardiac, endothelial, hematopoietic, and mesenchymal stem and progenitor cells to the infarcted area, while also promoting angiogenesis and cardiomyocyte survival. IL-10 reduces inflammation by inhibiting the infiltration of immune cells into the myocardium and also reduces cardiomyocyte death. TIMP-3 helps preserve the cardiac ECM structure by inhibiting the activity and reducing the expression levels of MMPs and also promotes anti-inflammatory activities and cardiomyocyte survival.

FIG. 17A. The release system was comprised of a fibrin gel embedding TIMP-3 and IL-10 aimed for early release; and FGF-2-loaded and SDF-1α-loaded coacervate droplets distributed within the same gel aimed for late release. The coacervate was formed through electrostatic interactions by combining FGF-2 or SDF-1α with heparin then with PEAD polycation. FIG. 17B. The release system described achieved sequential quick release of TIMP-3 and IL-10 by one week followed by a sustained release of FGF-2 and SDF-1α up to six weeks. Data are presented as means±SD (n=3).

FIGS. 18A-18D. Fractional factorial design results. FIG. 18A. Factorial regression model shows the relative significance of each of the 4 proteins: TIMP-3, IL-10, FGF-2, SDF-1α, and some of the 2-way protein interactions on improvement of ejection fraction (EF %). FIG. 18B. The main effects plot shows the individual effect of each protein on EF % from respective lower to upper dosages. FIG. 18C. The interactions plot shows the three 2-way interactions that were computed. FIG. 18D. The interaction plot between TIMP-3 and FGF-2, although not significant, suggests slight antagonism between the 2 proteins.

FIG. 19A. Modified regression model, after removing IL-10, shows improved fit. FIG. 19B. A contour plot predicts value of EF % upon choosing dosages for TIMP-3 and FGF-2 among a range of values, while fixing SDF-1α dosage.

FIG. 20. The refined delivery approach includes injecting the infarcted heart with a fibrin gel-coacervate composite containing TIMP-3 within the fibrin gel, and FGF-2/SDF-1α-loaded coacervates distributed in the same gel, with proteins at 3 μg each.

FIG. 21A. Traces of end-systolic (ESA) and end-diastolic (EDA) areas from short-axis B-mode images of the left ventricle (LV) using echocardiography. FIG. 21B. Fractional area change (FAC) values show differences between groups after MI at multiple time points, with significantly higher FAC value of the delivery group compared to saline and free from 2 weeks on. FIG. 21C. Saline and free groups show increasing ESA values, which were reduced in delivery group. FIG. 21D. Saline and free groups show increasing EDA values, which were reduced in delivery group. Data are presented as means±SD (n=9-10 per group). * p<0.05 vs Saline, ≠ p<0.05 vs Free.

FIG. 22A. Traces of end-systolic (ESA) and end-diastolic (EDA) areas from short-axis view images of the left ventricle (LV) using cardiac MRI. FIG. 22B. Ejection fraction (EF) values show differences between groups after MI at 8 weeks, with significantly higher EF % of the delivery group compared to saline and free. FIG. 22C. Saline and free groups show increasing ESV value at 8 weeks, which was significantly reduced in delivery group. FIG. 22D. Saline and free groups show increasing EDV value at 8 weeks. Data are presented as means±SD (n=5-8 per group). * p<0.05 vs Saline, ≠ p<0.05 vs Free.

FIG. 23A. Strain of an infarcted sample was estimated by normalizing the estimated peak radial or circumferential strain in the infarcted area to that of the average of 4 non-infarct areas in LV walls during a cardiac cycle. FIG. 23B. Saline and free groups show decreasing radial strain at 8 weeks, which was significantly higher in delivery group. FIG. 23C. Saline and free groups show decreasing circumferential strain at 8 weeks, which was significantly higher in delivery group. Data are presented as means±SD (n=5 per group). * p<0.05 vs Saline.

FIG. 24A. Representative H&E images showed ventricular wall thinning with damaged cardiac muscle surrounded by scar tissue in saline and free proteins groups. However, these damages were apparently alleviated in the delivery group. Scale bar 1000 μm. FIG. 24B. Transition between collagenous scar tissue and healthy tissue at the borderzone of a non-treated infarct sample. FIG. 24C. Quantitative analysis shows generally reduced ventricular wall thinning by delivery group at 2 and 8 weeks over saline and free groups. Data are presented as means±SD (n=3-4/group at 2 wks, n=4-6 at 8 wks). * p<0.05 vs Saline.

FIG. 25. MMP-2/9 activity assay showed high levels of activity in infarct groups, but was significantly reduced in the delivery group compared to saline. Data are presented as means±SD (n=3-4 per group at 8 wks). * p<0.05 vs Saline.

FIG. 26A. Representative images of the different groups showing co-staining of F4/80, a pan-macrophage marker, and CD163, an M2 macrophage marker (green in original) at 2 weeks. Co-localization of the 2 markers shows the color as yellow (in original). FIG. 26B. The delivery group shows a reduced number of non-M2 macrophages compared to saline and free, but not statistically significant. FIG. 26C. The delivery group shows a significantly increased presence of M2 macrophages compared to saline. Data are presented as means±SD (n=3-4 per group at 2 wks). * p<0.05 vs Saline.

FIGS. 27A-27C. Representative images of the different groups showing staining of viable cardiac muscle by cardiac troponin I (cTnI) (green in original). Reduced viable muscle can be observed in all infarct groups, with better preservation of the muscle in the delivery group at 2 weeks (FIG. 27A) and at 8 weeks (FIG. 27B). FIG. 27C. Quantitative analysis shows no differences between infarct groups at 2 weeks, but demonstrates the delivery group's significant preservation of cardiac muscle viability at 8 weeks compared to saline. Data are presented as means±SD (n=3-5/group at 2 wks, n=5-6 at 8 wks). * p<0.05 vs Saline.

FIG. 28A. Representative Western blot images of the expression levels of p-ERK, p-Akt and cleaved caspase-3 in different study groups at 8 weeks. FIG. 28B. The intensity band analysis of cleaved caspase-3 shows significant reduction of expression level in delivery group compared to saline and free groups. FIG. 28C. The intensity band analysis of p-ERK1/2 shows significant increase of expression level in delivery group compared to saline and free groups, with free showing significance over saline as well. FIG. 28D. The intensity band analysis of p-Akt shows significant of expression level in delivery group compared to saline and free groups. Data are presented as means±SD (n=3/group at 8 wks). * p<0.05 vs Saline, ≠ p<0.05 vs Free.

FIG. 29A. Representative images of the infarct groups showing co-staining of vWF (red in original), an endothelial marker, and α-SMA (green in original), a pericyte marker at 8 weeks. FIG. 29B. The delivery group shows a significantly greater number of vWF+ vessels than saline at 2 weeks and than saline and free at 8 weeks. FIG. 29C. The delivery group shows a significantly greater number of vWF+ α-SMA+ vessels than saline and free groups at 8 weeks but not at 2 weeks. Data are presented as means±SD (n=3-4/group at 2 wks, n=5-6 at 8 wks). * p<0.05 vs Saline, ≠ p<0.05 vs Free.

FIG. 30A. Representative images of the infarct groups showing staining of c-Kit+ stem cells (green in original) at 8 weeks. FIG. 30B. Quantitative analysis shows significantly greater number of c-Kit+ stem cells in delivery group compared to saline and free groups. Data are presented as means±SD (n=5/group at 8 wks). * p<0.05 vs Saline, ≠ p<0.05 vs Free.

FIGS. 31A and 31B. Representative Picosirius red staining images, at 2 weeks (FIG. 31A, bar 1000 μm) and 8 weeks (FIG. 31B), show the dense collagen deposition along the LV wall and infarct zone in saline, followed by the free group, whereas it was limited to the infarct region in the delivery group. FIG. 31C. Quantitative analysis shows that collagen deposition was not different in infarct groups at 2 weeks but was significantly less in the delivery group compared to saline and free groups at 8 weeks. Data are presented as means±SD (n=3-5/group at 2 wks, n=4-7 at 8 wks). * p<0.05 vs Saline, ≠ p<0.05 vs Free.

FIG. 32. Expression levels of relevant proteins in tissue lysates at 8 weeks. FIG. 32A. Free and delivery groups significantly increased IGF-1 levels. FIG. 32B. Delivery group significantly increased VEGF levels compared to saline. FIG. 32C. Delivery group significantly increased Shh levels compared to saline. FIG. 32D. Free group significantly decreased TGF-β1 levels compared to saline, but delivery group significantly decreased TGF-β1 levels compared to both saline and free. Data are presented as means±SD (n=3-4/group at 8 wks). * p<0.05 vs Saline, ≠ p<0.05 vs Free.

FIGS. 33A and 33B provide cDNA sequences for TIMP3 (SEQ ID NO: 6) and CXCL2 (SEQ ID NO: 7), respectively.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

As used herein, the terms “comprising,” “comprise” or “comprised,” and variations thereof, are meant to be open ended. The terms “a” and “an” are intended to refer to one or more.

As used herein, the term “patient” or “subject” refers to members of the animal kingdom including but not limited to human beings.

A controlled delivery system is provided herein that controls spatiotemporal cues in order to release a first active agent according to a first release profile and a second active agent according to a second release profile. The first active agent is contained within a coacervate of a polycationic polymer, a polyanionic polymer, and the second active agent is contained within a bioerodible, biocompatible hydrogel. In one aspect, the polyanionic polymer is a biopolymer, such as heparin or heparan sulfate. Examples of suitable polycationic polymers are described below and include PEAD (poly(ethylene arginylaspartate diglyceride)) and PELD (poly(ethylene lysinylaspartate diglyceride)).

Other non-limiting examples of polyanionic biopolymers include: pectin, gellan (gellan gum), alginic acid, dextran sulfate, carrageenan, and xanthane.

As used herein, the term “polymer composition” is a composition comprising one or more polymers. As a class, “polymers” includes homopolymers, heteropolymers, co-polymers, block polymers, block co-polymers and can be both natural and synthetic. Homopolymers contain one type of building block, or monomer, whereas co-polymers contain more than one type of monomer.

The term “alkyl” refers to both branched and straight-chain saturated aliphatic hydrocarbon groups. These groups can have a stated number of carbon atoms, expressed as C_{x-y}, where x and y typically are integers. For example, C₅₋₁₀, includes C₅, C₆, C₇, C₈, C₉, and C₁₀. Alkyl groups include, without limitation: methyl, ethyl, propyl, isopropyl, n-, s- and t-butyl, n- and s-pentyl, hexyl, heptyl, octyl, etc. Alkenes comprise one or more double bonds and alkynes comprise one or more triple bonds. These groups include groups that have two or more points of attachment (e.g., alkylene). Cycloalkyl groups are saturated ring groups, such as cyclopropyl, cyclobutyl, or cyclopentyl. As used herein, “halo” or “halogen” refers to fluoro, chloro, bromo, and iodo.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone. A polymer is said to comprise a specific type of linkage, such as an ester, or urethane linkage, if that linkage is present in the polymer.

Certain polymers described herein, such as fibrin and PEAD, are said to be bioerodible or biodegradable. By that, it is meant that the polymer, once implanted and placed in contact with bodily fluids and tissues, or subjected to other environmental conditions, such as composting, will degrade either partially or completely through chemical reactions, typically and often preferably over a time period of hours, days, weeks or months. Non-limiting examples of such chemical reactions include acid/base reactions, hydrolysis reactions, and enzymatic cleavage. Certain polymers described herein contain labile ester linkages. The polymer or polymers may be selected so that it degrades over a time period. Non-limiting examples of useful in situ degradation rates include between 12 hours and 5 years, and increments of hours, days, weeks, months or years therebetween.

A drug delivery system is provided, comprising, a coacervate of a polycationic polymer, a polyanionic polymer, and a first active agent embedded within a bioerodible, biocompatible hydrogel comprising a second active agent. In certain embodiments, the polycationic described herein comprises the structure (that is, comprises the moiety: [—OC(O)—B′—CH(OR1)-B—]ₙ or —[OC(O)—B—C(O)O—CH₂—CH(O—R1)-CH₂—B′—CH₂—CH(O—R2)-CH₂—]ₙ, in which B and B′ are the same or different and are organic groups, or B′ is not present, including, but not limited to: alkyl, ether, tertiary amine, ester, amide, or alcohol, and can be linear, branched or cyclic, saturated or unsaturated, aliphatic or aromatic, and optionally comprise one or more protected active groups, such as, without limitation, protected amines and acids, and R1 and R2 are the same or different and are hydrogen or a functional group (e.g., as described herein). As seen below, the composition exhibits low polydispersity, with a polydispersity index of less than 3.0, and in many cases less than 2.0. These compositions are described in U.S. patent application Ser. No. 13/522,996, which is incorporated by reference in its entirety.

The polymers described herein can be functionalized, e.g., at B, B′, R1 and R2, meaning they comprise one or more groups with an activity, such as a biological activity. For example and without limitation, as shown herein, the polymer may be functionalized with an acetylcholine-like group or moiety, a cross-linking agent (cross-linking agents contain at least two reactive groups that are reactive towards numerous groups, including sulfhydryls and amines, and create chemical covalent bonds between two or more molecules, functional groups that can be targeted with cross-linking agents include primary amines, carboxyls, sulfhydryls, carbohydrates and carboxylic acids. A large number of such agents are available commercially from, e.g., Thermo fisher Scientific (Pierce) and Sigma).

Other functions that can be provided by or enhanced by addition of functional groups include: increased hydrophobicity, for instance by functionalizing with a superhydrophobic moiety, such as a perfluoroalkane, a perfluoro(alkylsilane), and/or a siloxane; increased hydrophilicity, for instance by functionalizing with polyethylene glycol (PEG); anticoagulation, for instance, by functionalizing with heparin; or antimicrobial, for instance, by functionalizing with a quaternary amine. The polymer can be functionalized with a tag, such as a fluorescent tag (e.g., FITC, a cyanine dye, etc.). The polymer can be functionalized by linking to additional synthetic or natural polymers, including, without limitation: synthetic polymers, such as a polymer derived from an alpha-hydroxy acid, a polylactide, a poly(lactide-co-glycolide), a poly(L-lactide-co-caprolactone), a polyglycolic acid, a poly(dl-lactide-co-glycolide), a poly(l-lactide-co-dl-lactide), a polymer comprising a lactone monomer, a polycaprolactone, a polymer comprising carbonate linkages, a polycarbonate, a polyglyconate, a poly(glycolide-co-trimethylene carbonate), a poly(glycolide-co-trimethylene carbonate-co-dioxanone), a polymer comprising urethane linkages, a polyurethane, a poly(ester urethane) urea, a poly(ester urethane) urea elastomer, a polymer comprising ester linkages, a polyalkanoate, a polyhydroxybutyrate, a polyhydroxyvalerate, a polydioxanone, a polygalactin, or natural polymers, such as chitosan, collagen, elastin, alginate, cellulose, hyaluronic acid and gelatin.

The compositions may be functionalized with organic or inorganic moieties to achieve desired physical attributes (e.g., hardness, elasticity, color, additional chemical reactivity, etc.), or desired functionality. For example, the polymer composition may be derivatized with maleic acid or phosphate.

The composition is formed into a coacervate with active agents or polyanionic or polycationic groups to for sequestering active agents for controlled delivery in vivo. Drug products comprising the derivatized polyester compounds described herein may be delivered to a patient by any suitable route of delivery (e.g. oral or parenteral), or even as an implantable device for slow release of the active agent.

The functional groups may vary as indicated above. For example, in certain embodiments, R1 and R2 are independently groups comprising acetylcholine, a carboxy-containing group, an α, β unsaturated carboxylic acid (such as cinnamic group (e.g., functionalized with cinnamic acid, p-coumaric acid, ferulic acid, caffeic acid); an amine-containing group, a quaternary ammonium containing group, maleic acid, a peptide; maleate; succinate or phosphate, halo-containing groups. In one embodiment, one or more of B, B′, R1 and R2 are charged such that it is possible to bind various water insoluble organic or inorganic compounds to the polymer, such as magnetic inorganic compounds. As above, in one embodiment, one or more of B, B′, R1 and R2 are positively charged. In one embodiment, one or both of R1 and R2 are functionalized with a phosphate group. In another embodiment, the composition is attached non-covalently to a calcium phosphate (including as a group, for example and without limitation: hydroxyapatite, apatite, tricalcium phosphate, octacalcium phosphate, calcium hydrogen phosphate, and calcium dihydrogen phosphate). In yet another embodiment, R1 and R2 are independently one Ile-Lys-Val-Ala-Val (IKVAV) (SEQ ID NO: 8), Arg-Gly-Asp (RGD), Arg-Gly-Asp-Ser (RGDS) (SEQ ID NO: 9), Ala-Gly-Asp (AGD), Lys-Gin-Ala-Gly-Asp-Val (KQAGDV) (SEQ ID NO: 10), Val-Ala-Pro-Gly-Val-Gly (VAPGVG) (SEQ ID NO: 11), APGVGV (SEQ ID NO: 12), PGVGVA (SEQ ID NO: 13), VAP, GVGVA (SEQ ID NO: 14), VAPG (SEQ ID NO: 15), VGVAPG (SEQ ID NO: 16), VGVA (SEQ ID NO: 17), VAPGV (SEQ ID NO: 18) and GVAPGV (SEQ ID NO: 19)). In specific embodiments, the composition may be PSeD, functionalized PSeD.

In another aspect, B and B′ are residues of aspartic acid or glutamic acid, which are optionally further derivatized with an amine-containing group, for example, the amines of the aspartic acid or glutamic acid are further derivatized with lysine or arginine. Examples of such compositions include:
(a) [—OC(O)—CH(NHY)—CH₂—C(O)O—CH₂—CH(O—R1)-CH₂—O—CH₂—CH₂—O—CH₂—CH(O—R2)-CH₂]ₙ,
(b) [—OC(O)—CH₂—CH(NHY)—C(O)O—CH₂—CH(O—R1)-CH₂—O—CH₂—CH₂—O—CH₂—CH(O—R2)-CH₂—]ₙ,
(c) [—OC(O)—CH(NHY)—CH₂—CH₂—C(O)O—CH₂—CH(O—R1)-CH₂—O—CH₂—CH₂—O—CH₂—CH(O—R2)-CH₂—]ₙ, and/or
(d) [—OC(O)—CH₂—CH₂—CH(NHY)—C(O)O—CH2-CH(O—R1)-CH₂—O—CH₂—CH₂—O—CH₂—CH(O—R2)-CH₂—]ₙ,
   wherein Y is —C(O)—CH(NH₃⁺)—(CH₂)₃—NH—C(NH₂)₂⁺ or —C(O)—CH(NH₃⁺)—(CH₂)₄—(NH₃)⁺, and R1 and R2 are the same or different and are independently selected from the group consisting of hydrogen, acetylcholine, a carboxy-containing group, an α, β unsaturated carboxylic acid, a cinnamic acid containing group, a p-coumaric acid containing group, a ferulic acid containing group, a caffeic acid containing group, an amine-containing group, a quaternary ammonium containing group, maleic acid, a peptide, maleate, succinate, a phosphate-containing group, and a halo-containing group. Examples of the copolymer composition (a), (b), (c), and/or (d), above include:
   PEAD (poly(ethylene arginylaspartate diglyceride)), having the structure:

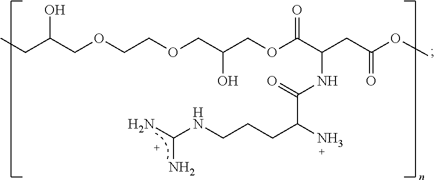

PELD (poly(ethylene lysinylaspartate diglyceride)), having the structure:

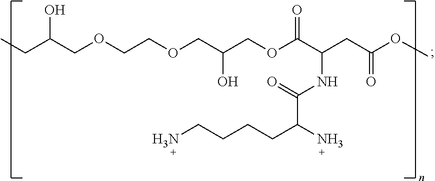

(poly(ethylene arginylglutamate diglyceride)), having the structure:

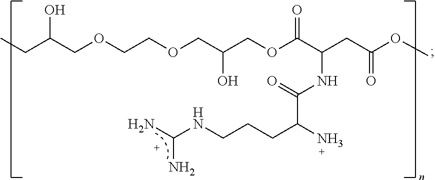

(poly(ethylene lysinylglutamate diglyceride)), having the structure:

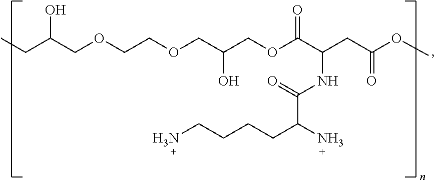

where n is >1, and one or both of the —OH groups are optionally modified with a functional group, for example: acetylcholine, a carboxy-containing group, an α, β unsaturated carboxylic acid, a cinnamic acid containing group, a p-coumaric acid containing group, a ferulic acid containing group, a caffeic acid containing group, an amine-containing group, a quaternary ammonium containing group, maleic acid, a peptide, maleate, succinate, a phosphate-containing group, and a halo-containing group. Synthesis of PEAD is described below in the examples. Synthesis of the lysinyl/arginyl and glutamate/aspartate variations, e.g., as described above, is performed similarly, and synthesis of variations thereof, such as functionalized versions thereof, would be well within the skill of an ordinary artisan.

In forming the coacervate, the cationic polycationic polymer is complexed with a polyanionic polymer, such as heparin or heparan sulfate, and an active agent, such as a growth factor, small molecule, cytokine, drug, a biologic, a protein or polypeptide, a chemoattractant, a binding reagent, an antibody or antibody fragment, a receptor or a receptor fragment, a ligand, or an antigen and/or an epitope. In one aspect, the active agent is first combined with the polyanionic polymer, such as heparin, and then the polycationic polymer is added to form the coacervate. The coacervate is then embedded into a biocompatible, bioereodible hydrogel, such as a fibrin hydrogel. In one embodiment, the polycationic composition comprises a coacervate of a polycationic polymer comprising one or more of moieties (a), (b), (c), and/or (d), as described above, and further comprising heparin or heparin sulfate complexed (that is non-covalently bound) with the a first active agent, such as PDGF, and embedded in a hydrogel matrix of fibrin comprising mixed therein VEGF.

The compositions described herein are useful for delivery of a large variety of active agents. Biologics as a class include, for example, carbohydrates, proteins, or nucleic acids or complex combinations of these substances, including oligopeptide and polypeptide active agents such as growth factors, cytokines, antibodies and antibody fragments. Cellular biologicals also can be delivered by the compositions and methods described herein. Other active agents, e.g. large and small molecule compounds and particles, are equally amenable to delivery by the compositions and methods described herein.

Active agents that may be incorporated into the coacervate and/or the bioerodible, biocompatible hydrogel polymer include, without limitation, anti-inflammatories, such as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, antiinflammatory cytokines, and antiinflammatory proteins or steroidal anti-inflammatory agents); antibiotics; anticlotting factors such as heparin, Pebac, enoxaprin, aspirin, hirudin, plavix, bivalirudin, prasugrel, idraparinux, warfarin, coumadin, clopidogrel, PPACK, GGACK, tissue plasminogen activator, urokinase, and streptokinase; growth factors. Other active agents include, without limitation: (1) immunosuppressants; glucocorticoids such as hydrocortisone, betamethisone, dexamethasone, flumethasone, isoflupredone, methylprednisolone, prednisone, prednisolone, and triamcinolone acetonide; (2) antiangiogenics such as fluorouracil, paclitaxel, doxorubicin, cisplatin, methotrexate, cyclophosphamide, etoposide, pegaptanib, lucentis, tryptophanyl-tRNA synthetase, retaane, CA4P, AdPEDF, VEGF-TRAP-EYE, AG-103958, Avastin, JSM6427, TG100801, ATG3, OT-551, endostatin, thalidomide, becacizumab, neovastat; (3) antiproliferatives such as sirolimus, paclitaxel, perillyl alcohol, farnesyl transferase inhibitors, FPTIII, L744, antiproliferative factor, Van 10/4, doxorubicin, 5-FU, Daunomycin, Mitomycin, dexamethasone, azathioprine, chlorambucil, cyclophosphamide, methotrexate, mofetil, vasoactive intestinal polypeptide, and PACAP; (4) antibodies; drugs acting on immunophilins, such as cyclosporine, zotarolimus, everolimus, tacrolimus and sirolimus (rapamycin), interferons, TNF binding proteins; (5) taxanes, such as paclitaxel and docetaxel; statins, such as atorvastatin, lovastatin, simvastatin, pravastatin, fluvastatin and rosuvastatin; (6) nitric oxide donors or precursors, such as, without limitation, Angeli's Salt, L-Arginine, Free Base, Diethylamine NONOate, Diethylamine NONOate/AM, Glyco-SNAP-1, Glyco-SNAP-2, (.+-.)-S-Nitroso-N-acetylpenicillamine, S-Nitrosoglutathione, NOC-5, NOC-7, NOC-9, NOC-12, NOC-18, NOR-1, NOR-3, SIN-1, Hydrochloride, Sodium Nitroprusside, Dihydrate, Spermine NONOate, Streptozotocin; and (7) antibiotics, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, and silver salts such as chloride, bromide, iodide and periodate.

Further examples of active agents include: basic fibroblast growth factor (bFGF or FGF-2), acidic fibroblast growth factor (aFGF), nerve growth factor (NGF), vascular endothelial growth factor (VEGF), hepatocyte growth factor (HGF), insulin-like growth factors (IGF), transforming growth factor-beta pleiotrophin protein, midkine protein, platelet-derived growth factor (PDGF) and angiopoietin-1 (Ang-1). Active agents are included in the delivery system described herein, and are administered in amounts effective to achieve a desired end-point, such as angiogenesis, tissue growth, inhibition of tissue growth, or any other desirable end-point.

According to one embodiment, the delivery system and methods described herein are useful for promoting specific tissue growth. As an example, a differentiation factor is embedded into the bioerodible, biocompatible hydrogel, e.g. fibrin gel, for early release and a proliferation factor is within the coacervate, for delayed release. For each indicated purpose it is noted that appropriate relative amounts of the coacervate and bioerodible, biocompatible hydrogel may be used, as well as including effective amounts of the active agents for the intended purpose, respectively in the coacervate and bioerodible, biocompatible hydrogel. Appropriate and effective amounts of each component can be determined in the ordinary course by a person of skill in the art.

In one aspect, the active agents are, independently, one or more active agents selected from the group consisting of: VEGF, HGF, PDGF, TIMP-3, FGF-2, SDF-1α, IL-10, Ang1, Ang2, IGF-1, relaxin, Shh, FGF-1, NRG-1, BMP-2. Examples of useful active agent combinations for treatment of certain conditions follow in Table 1.

TABLE 1

| | | In bioerodible, biocompatible |
|---|---|---|
| | In coacervate | hydrogel |
| Action/condition | (e.g. PEAD + heparin) | (e.g., fibrin) |
| ischemia | PDGF | VEGF |
| ischemia | PDGF + Ang1 | VEGF + Ang2 |
| ischemia | HGF | IGF-1 |
| Bone regeneration | BMP-2 | VEGF |
| Bone regeneration | IGF-1 | BMP-2 |
| Bone regeneration | BMP-7 | BMP-2 |
| Tissue Growth | IGF-1 | TGF-β |

Each of PDGF, VEGF, Ang1 (also, ANGPT1, angiopoietin 1), Ang2 (also, ANGPT2, angiopoietin 2), HGF (hepatocyte growth factor), IGF-1 (insulin-like growth factor 1), BMP-2 (bone morphogenetic protein 2), TIMP-3, SDF-1α, FGF-2, IL-10 (Interleukin 10), NRG-1 (neuregulin 1), relaxin, Shh (sonic hedgehog), and FGF-1 (fibroblast Growth Factor 1) are broadly-known, well-characterized, and are available commercially, for example, from R&D Systems of Minneapolis, Minn. The following growth factors are employed in the examples below, and therefore exemplary amino acid sequences are provided.

TIMP3” is an acronym for tissue inhibitor of metalloproteinase 3 encoded by the TIMP3 gene. The protein sequences of TIMP3 of many vertebrates has been sequenced and are broadly-known, as are the cDNA sequences. In humans (OMIM 188826), TIMP3 has the protein sequence (UniProtKB-P35625 (TIMP3_HUMAN)): MTPWLGLIVL LGSWSLGDWG AEACTCSPSH PQDAFCNSDI VIRAKVVGKK LVKEGPFGTL VYTIKQMKMY RGFTKMPHVQ YIHTEASESL CGLKLEVNKY QYLLTGRVYD GKMYTGLCNF VERWDQLTLS QRKGLNYRYH LGCNCKIKSC YYLPCFVTSK NECLWTDMLS NFGYPGYQSK HYACIRQKGG YCSWYRGWAP PDKSIINATD P (SEQ ID NO: 1). Recombinant TIMP3 is available commercially from R&D Systems, of Minneapolis, Minn.

“FGF-2” refers to Fibroblast Growth Factor-2. FGF-2, including human FGF-2, is broadly-known and is broadly-available commercially, for example from PeproTech of Rocky Hill, N.J., with the amino acid sequence: AAGSITTLPA LPEDGGSGAF PPGHFKDPKR LYCKNGGFFL RIHPDGRVDG VREKSDPHIK LQLQAEERGV VSIKGVCANR YLAMKEDGRL LASKCVTDEC FFFERLESNN YNTYRSRKYT SWYVALKRTG QYKLGSKTGP GQKAILFLPM SAKS (SEQ ID NO: 2).

“SDF-1α” refers to Stromal-Cell Derived Factor-1, alpha isoform. It is encoded in humans by CXCL12 (OMIM 600835), having, e.g., the sequence (UniProtKB-P48061 (SDF1_HUMAN)): MNAKVVVVLV LVLTALCLSD GKPVSLSYRC PCRFFESHVA RANVKHLKIL NTPNCALQIV ARLKNNNRQV CIDPKLKWIQ EYLEKALNK (SEQ ID NO: 3). Recombinant SDF-1α is available commercially from PeproTech of Rocky Hill, N.J.

“PDGF” refers to platelet-derived growth factor, e.g., PDGF-BB, for example, SLGSLTIAEP AMIAECKTRT EVFEISRRLI DRTNANFLVW PPCVEVQRCS GCCNNRNVQC RPTQVQLRPV QVRKIEIVRK KPIFKKATVT LEDHLACKCE TVAAARPVT (SEQ ID NO: 4).

“VEGF” refers to vascular endothelial growth factor, e.g., VEGF₁₆₅, for example APMAEGGGQN HHEVVKFMDV YQRSYCHPIE TLVDIFQEYP DEIEYIFKPS CVPLMRCGGC CNDEGLECVP TEESNITMQI MRIKPHQGQH IGEMSFLQHN KCECRPKKDR ARQENPCGPC SERRKHLFVQ DPQTCKCSCK NTDSRCKARQ LELNERTCRC DKPRR (SEQ ID NO: 5)) are broadly-known and are broadly-available commercially.

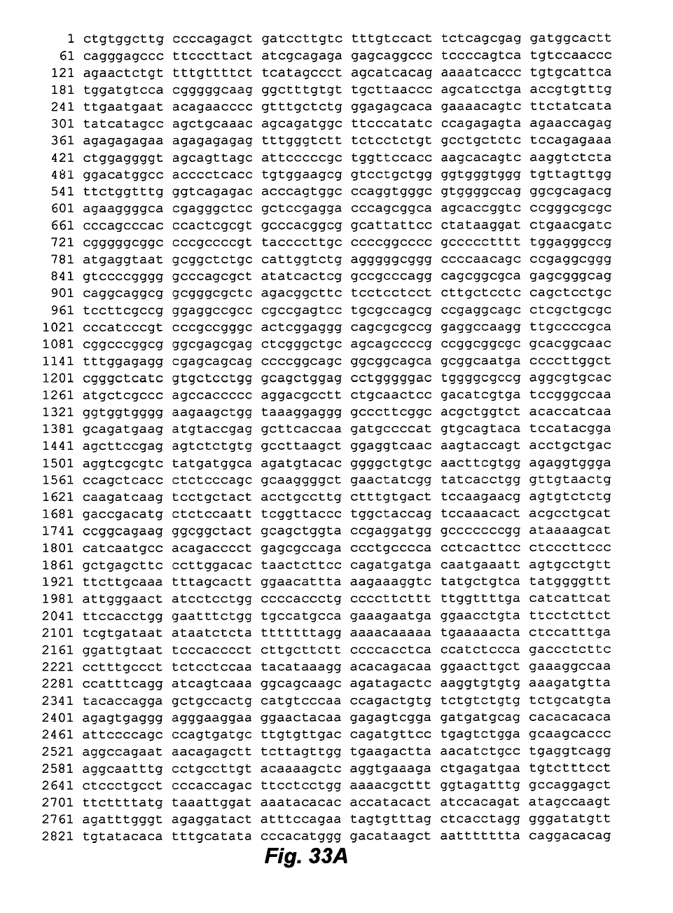

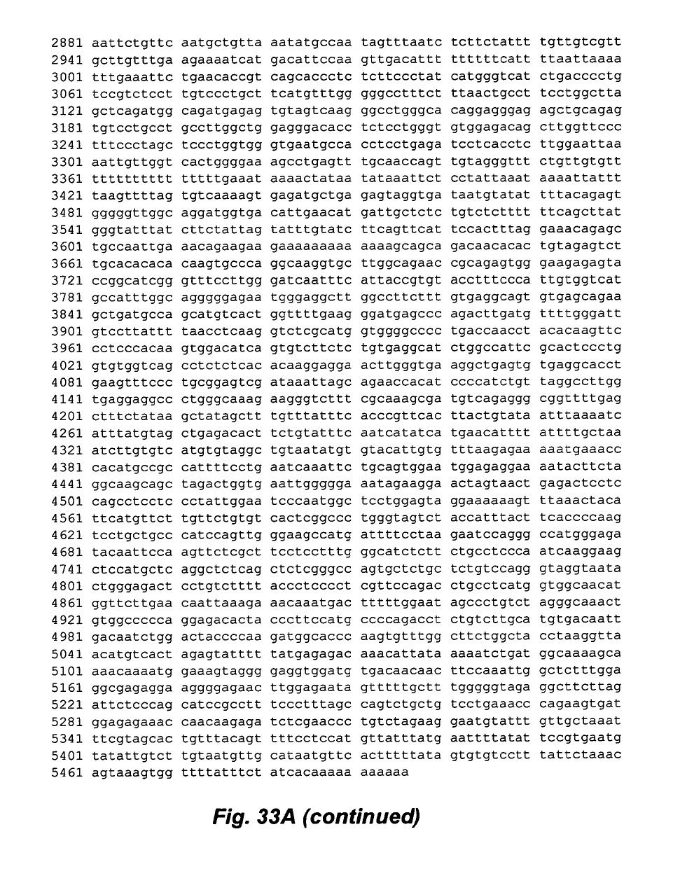

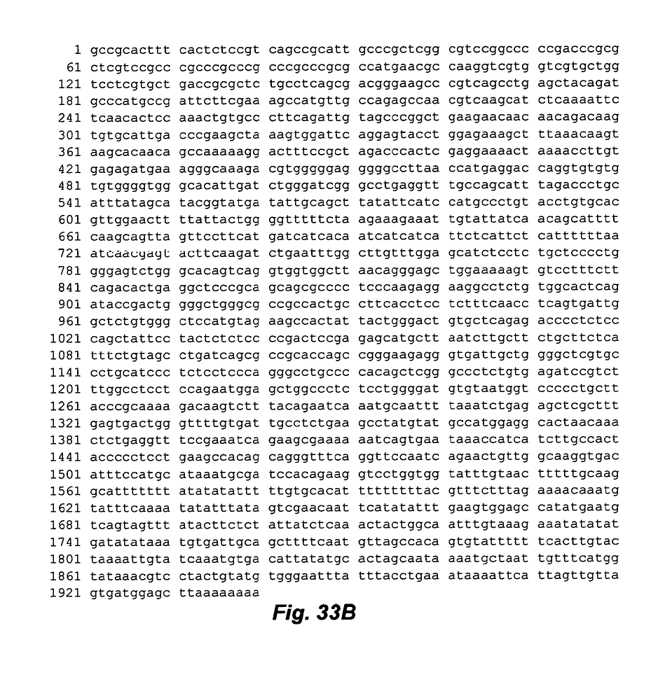

In reference to all polypeptide active agents described herein, for use in the compositions and methods described herein, variants, such as xenogeneic forms or modified recombinant forms, may be described and/or may be commercially available. Though it may be preferable in certain instances to use an allogeneic protein sequence, e.g., a human sequence for treatment of a human, xenogeneic polypeptides may prove acceptably effective, along with recombinant forms of the described polypeptide. As such, “PDGF”, “VEGF”, “TIMP3”, “FGF-2”, and “SDF-1α” refer to any functional form of PDGF, VEGF, TIMP3, FGF-3, or SDF-1α, including allogencic and xenogeneic variants, including recombinant versions thereof, so long as the activity is retained for the desired use of the polypeptide. Using polypeptide sequences, such as those provided herein, as well as the wealth of knowledge that is publicly available regarding any polypeptide described herein, one of ordinary skill, using standard, and well-established cloning and recombinant protein production methods can readily produce the described polypeptides by recombinant methods. For example, using either a codon table or a publicly-available cDNA sequence, DNA sequences encoding a polypeptide can be produced or obtained, such as the human TIMP3 cDNA sequence of GenBank Accession No. NM_000362 (Homo sapiens TIMP metallopeptidase inhibitor 3 (TIMP3), mRNA)(FIG. 33A SEQ ID NO: 6), or the human CXCL12 cDNA sequence of GenBank Accession No. NM_199168 (Homo sapiens chemokine (C-X-C motif) ligand 12 (CXCL12), transcript variant 1, mRNA, (FIG. 33B SEQ ID NO: 7)).

Coacervation as a process during which a homogeneous aqueous solution of charged macromolecules, undergoes liquid-liquid phase separation, giving rise to a polyelectrolyte-rich dense phase. To form a coacervate, the overall charge of the coacervate approaches neutral. Complex coacervation of polyelectrolytes can be achieved through electrostatic interaction with oppositely charged proteins or polymers. The charges on the polyelectrolytes must be sufficiently large to cause significant electrostatic interactions, but not so large to cause precipitation. For coacervates, the zeta potential of the aggregated elements of the coacervate, for example in the context of the present disclosure, the mixture of the polycationic polymer, the polyanionic polymer and any active agent, approaches zero, for example, ranging from −15 mV to 15 mV, e.g., from −10 to 10 mV, or from −5 mV to 5 mV. In any aspect of the compositions and methods described herein, the amount of each active agent incorporated into the gel is an amount effective to achieve a desired end-point, such as heart tissue repair or bone growth without unacceptable toxicity or other harmful sequelae in a patient.

The coacervate/hydrogel composition described herein can be used in a variety of ways. In one aspect, it can find use in vitro or ex vivo in cell, organ or tissue culturing methods, e.g., for delivery of growth factors, cytokines, antibiotics, etc. The compositions are injected, or otherwise placed in an anatomical site suitable for the desired treatment, for example at the site of an infarct. The composition can have any useful physical size and shape in vitro, and can be molded into such shapes by standard methods. Likewise, for in vivo uses, the composition also can have any useful physical size and shape in vitro, and can be molded into such shapes by standard methods. Alternatively, the composition can be delivered in vivo as an injectable. As illustrated in the Examples below, the coacervate is formed and is mixed with the hydrogel composition, and is delivered prior to gelation of the hydrogel, such that the hydrogel is injected as a bolus while a solution, and forms a gel in situ (where it is injected), for example in the heart. In the examples below, the coacervate is mixed with fibrinogen, and is admixed with thrombin immediately before delivery in vivo by injection such that the composition is injected as a solution and forms a fibrin-coacervate mass in situ. Proteinase inhibitors, such as aprotonin, can also be mixed with the fibrinogen to prevent premature degradation of a proteinaceous hydrogel component of the coacervate-hydrogel composition, e.g., fibrinogen and/or fibrin prior to and during injection and gelation. Other illustrative examples of useful hydrogels include, without limitation: collagen, gelatin, chitosan, alginate, hyaluronic acid, PEG (poly(ethylene glycol), starch, agarose, pectin, silica, and PVA (poly(vinyl alcohol)).

In one aspect, the composition described herein, for example a coacervate-hydrogel composition comprising PDGF and VEGF, or the combination of TIMP3, FGF-2, and SDF-1α, essentially as described herein, e.g., in the Examples below, is useful for treatment of an ischemic event, for example for treatment of a myocardial infarct. For treatment of an ischemic event, for example a myocardial infarction, the composition is injected at one or more sites in or adjacent to an infarct during or after an ischemic event. The compositions can be administered more than once, but due to the temporal nature of the delivery process, additional administrations of the composition typically will take place after any active agent in the previously administered coacervate is released.

The compositions described herein can be packaged, stored, and distributed in any useful manner. According to one aspect of the invention, a kit is provided, comprising a coacervate-hydrogel composition, as described herein in a vessel. In one aspect, the kit comprises to coacervate in a hydrogel precursor in a vessel that is in a solution state, and gels on injection in vivo, typically with mixture with an activator, provided in a second vessel. For example, the kit comprises a coacervate mixed in solution with fibrinogen in one vessel and thrombin in a second vessel.

### Example 1—a Biocompatible Heparin-Binding Polycation as a Growth Factor Delivery Vehicle

### Methods

### Synthesis of PEAD—

t-BOC protected aspartic acid (t-BOC Asp), t-BOC protected arginine (t-BOC-Arg) (EMD Chemicals, NJ), ethylene glycol diglycidyl ether (EGDE), trifluoroacetic acid (TFA) (TCI America, OR), anhydrous 1,4-dioxane and tetra-n-butylammonium bromide (TBAB) (Acros organics, Geel, Belgium), dicyclohexylcarbodiimide (DCC), N-hydroxysuccinimide (NHS) (Alfa Aesar, MA) and 4-dimethylaminopyridine (DMAP) (Avocado Research Chemicals Ltd, Lancaster, UK) were used for PEAD synthesis without purification.

The synthesis of PEAD was performed as follows. EGDE and t-BOC Asp were polymerized in 1,4-dioxane under the catalysis of TBAB. t-BOC protection was later removed by TFA to generate primary amine. t-BOC-Arg was conjugated by DCC/NHS/DMAP coupling followed by the second deprotection to yield PEAD. The chemical structure was confirmed using NMR and FT-IR. The molecular weight of PEAD was measured by PL-GPC 50 Plus-RI equipped with a PD 2020 light scattering detector (Varian, MA). Two MesoPore 300×7.5 mm columns and 0.1% of LiBr in DMF were used as solid phase and mobile phase, respectively.

### Zeta Potential Measurement—

All polymer solution was prepared in pure water at the concentration of 1 mg/ml. 100 μl of heparin (Alfa Aesar, MA) solution was mixed with different volume of PEAD solution. The mixture was diluted in 1 ml of water then 750 μl of solution was taken for the measurement. Zeta potential was measured by Zetasizer Nano Z (Malvern, MA). The results showed the average of measurement (n=30).

### Dimethylmethylene Blue Assay—

Dimethylmethylene blue (DMB) has been used for quantification of sulfated glycosaminoglycan in the solution (de Jong, J. G., R. A. Wevers, et al. (1989). “Dimethylmethylene blue-based spectrophotometry of glycosaminoglycans in untreated urine: a rapid screening procedure for mucopolysaccharidoses.” Clinical Chemistry 35(7): 1472-1477 and DeBlois, C., M.-F. Côté, et al. (1994). “Heparin-fibroblast growth factorfibrin complex: in vitro and in vivo applications to collagen-based materials.” Biomaterials 15(9): 665-672). Briefly, 20 μl of heparin solution (1 mg/ml) was mixed with different volume of PEAD solution (1 mg/ml). H₂O was added to the complex solution to let the final volume become 200 μl. After centrifuge at 13,400 rpm for 5 min, 50 μl of supernatant was taken to interact with DMB working solution, 10.7 μg of 1,9-dimethylmethylene blue chloride (Polysciences, PA) in 55 mM formic acid. A series of standard solutions containing known concentrations of heparin were used to make a standard curve. The absorbance at 520 nm was determined.

### Scanning Electron Microscopy—

The SEM samples were prepared by mixing PEAD with heparin (mass ratio 5) to form the complex. The complex were dropped on an aluminum stub, sputtered with gold then viewed with Leo 1530 SEM (10 kV, 3 nm spot size).

### Growth Factor Loading Efficiency—

PEAD and heparin were dissolved in saline to prepare 10 mg/ml solution. 100 or 500 ng of the unlabeled growth factor was mixed with the 125I-labeled growth factor followed by addition of 10 μl of heparin solution then 50 μl of PEAD solution. The growth factor loaded PEAD/heparin complex was precipitated by centrifugation at 13,400 rpm for 5 min. The supernatant was collected for radioactivity measurement by the gamma counter.

For loading efficiency determined by enzyme-linked immunosorbent assay (ELISA), two different methods were adopted, indirect and sandwich ELISA. For FGF-2, PEAD/heparin-complexed FGF-2 was coated onto a 96-well plate for overnight. An anti-FGF-2 polyclonal antibody (PeproTech, NJ) was used for recognition. For NGF, a sandwich ELISA was conducted using NGF Emax® ImmunoAssay Systems (Promega, WI).

### Growth Factor Release Profile—

After the removal of the supernatant for testing the loading efficiency, 500 μl of saline was added to cover the pellet. At different time points (day 1, 4, 7, 14, 19, 28, 33 and 42), the supernatant was collected and fresh saline was filled again. The radioactivity of the collected supernatant was measured to determine the amount of the growth factor released.

### FGF-2 Bioactivity—

FGF-2 bioactivity was determined by its stimulation of human aortic endothelial cells (HAECs) proliferation. Briefly, HAECs were cultured on a 24-well plate with MCDB 131 containing 10% fetal bovine serum (FBS), 1% L-glutamine and 50 μg/ml ascorbic acid. A cell culture insert (BD Biosciences, MA) with pore size 1.0 μm was placed on each well. Bolus FGF-2 or PEAD/heparin-complexed FGF-2 100 ng was added into the insert (Chen, P.-R., et al. (2005). “Release characteristics and bioactivity of gelatin-tricalcium phosphate membranes covalently immobilized with nerve growth factors.” Biomaterials 26(33): 6579-6587). Endothelial cell (EC) culture supplement (Sigma, MO) included 1 ng/ml epithelial growth factor (EGF), 2 ng/ml FGF-2, 2 ng/ml insulin-like growth factor-1 (IGF-1), 1 ng/ml vascular endothelial growth factor (VEGF) and 1 μg/ml hydrocortisone. After culturing for 4 days, the proliferation of HAEC was determined by CyQuant Cell Proliferation Assays (Invitrogen, CA). All results were normalized to the control group which has no supplemental growth factors.

### NGF Bioactivity—

NGF bioactivity was determined by its stimulation of the differentiation of PC-12 cells. PC-12 cells were maintained in Dulbecco's Modified Eagle Medium (DMEM) supplemented with 1.0% horse serum (HS) and 0.5% FBS. Bolus NGF, heparin-stabilized NGF or PEAD/heparin-complexed NGF 10 ng was added into the cell culture insert. On day 4 and day 7, the phase contrast images were taken. The neurite lengths were measured using NIH ImageJ version 1.42. The longest ten neurites were shown as the average value along with the standard deviation.

### Statistical Analysis—

Student's t-test was used as a statistical tool to analyze the bioactivity of FGF-2 and NGF. p value <0.05 was marked a significant difference. Data represent mean±SD.

### Results

The synthesis of PEAD initiated from the polycondensation of aspartic acid and ethylene glycol diglycidyl ether (EGDE) followed by the conjugation of arginine which provides positive charges for the polymer. PEAD has +2 charges per monomer at the physiological condition owning to the primary amino group and the guanidinium group of arginine (FIG. 1). The weight-average molecular weight (Mw) is 30,337 Da with polydispersity index (PDI) 2.28.

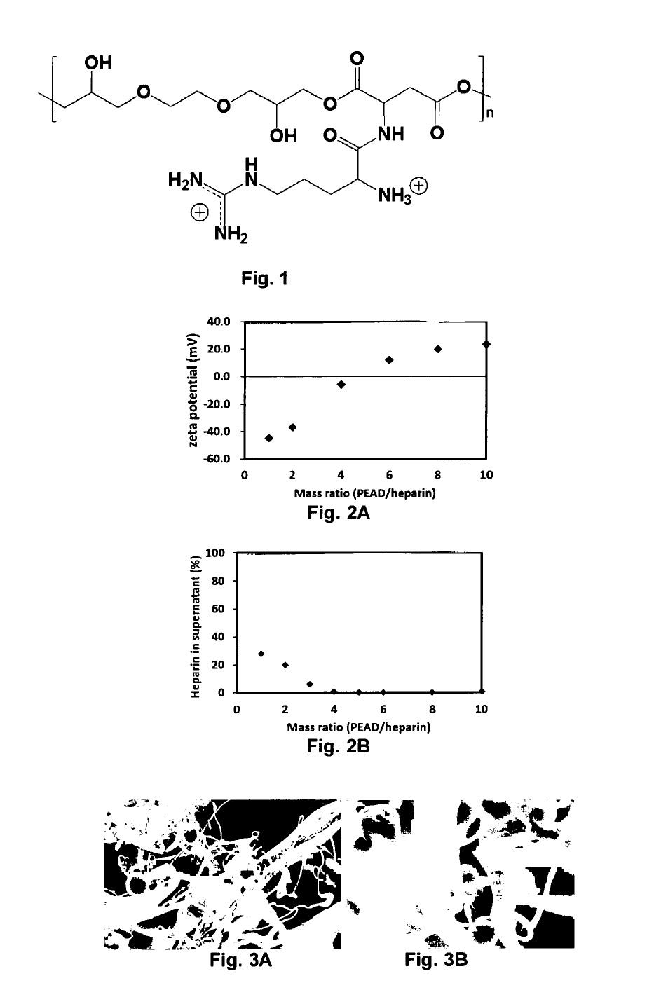

To test the binding ability of PEAD to heparin, zeta potential measurement was performed. The result (FIG. 2A) shows with the increase of the mass ratio of PEAD to heparin, the zeta potential of the complex shifted from negatively-charged (−45 mV) at ratio 1 to positively-charged (+23.2 mV) at ratio 10. Continuing adding more PEAD did not change the zeta potential and +23.2 mV is close to the zeta potential of PEAD itself. It suggests after ratio 10 the complex was all covered by PEAD. Besides it also shows at ratio 5 PEAD almost neutralized all negative charges of heparin. From the macroscopic observation, below ratio 5 the addition of PEAD let the heparin solution became more turbid and precipitate was seen after a few minutes. Whereas the ratio was over 5, the addition of PEAD would let the solution become clear again.

Further confirming the binding ability, we mixed different amounts of PEAD to heparin solutions then precipitated the complex by centrifugation. Because the neutralization of the negative-charged heparin favors the formation of precipitate, we measured the amount of heparin left in the supernatant to determine the binding affinity between PEAD and heparin. For this assay, we applied a heparin binding dye, dimethylmethylene blue (DMB) to detect free heparin by measuring the absorption of DMB at 520 nm. The result (FIG. 2B) shows the amount of heparin in the supernatant was gradually lowered with the addition of PEAD. When the ratio of PEAD to heparin is over 3, >90% of heparin was precipitated through centrifugation. At the ratio 5, that would be >99% of heparin. This result has a good correlation with that of zeta potential measurement because both experiments suggest at ratio 5 PEAD and heparin has the maximum interaction. Therefore this ratio was used for the remaining experiment.

According to the chemical structure, PEAD has a linear backbone connected to positively-charged brushes, arginine, to interact with heparin. To understand the morphology of PEAD/heparin complex, we took pictures under scanning electron microscope (SEM) (FIGS. 3A and 3B). The pictures reveal the morphology of PEAD/heparin complex has fibrous structure with many small globular domains. The diameter of fibers covers a wide range from m to nm.

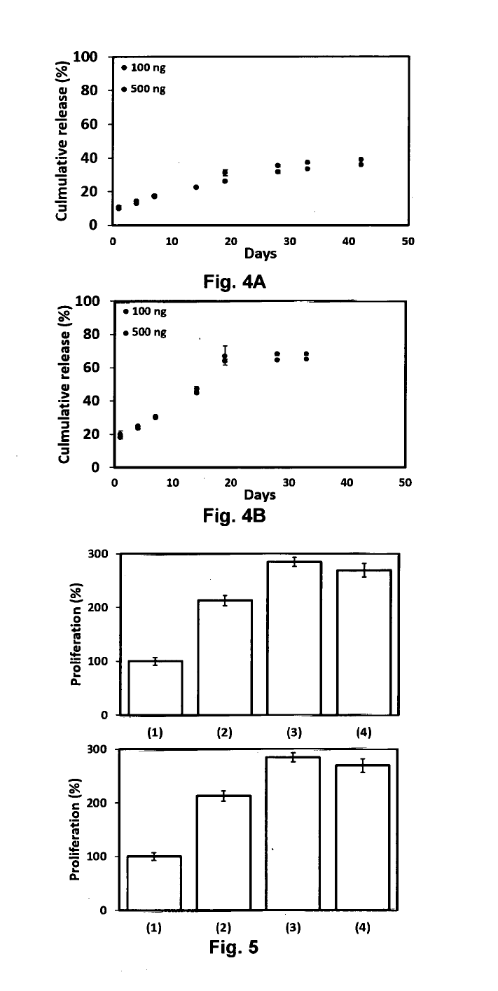

It is understood that a variety of growth factors can bind to heparin with the dissociation constant (Kd) from μM to nM. The loading efficiency of growth factors to PEAD/heparin complex was examined. In this experiment 100 or 500 ng of fibroblast growth factor-2 (FGF-2) plus 125I-labeled FGF-2 used as a tracer were mixed with heparin then added into PEAD solution. After staying at room temperature for 2 hr, centrifugation was used to precipitate PEAD/heparin/FGF-2. The amount of unloaded FGF-2 remaining in the supernatant can be determined by a gamma counter. The result (Table 2) shows PEAD/heparin loaded ˜68% of FGF-2 for both high and low amounts of FGF-2. The other growth factor, NGF, the release (FIG. 4B) is clearly faster. The initial burst reached almost 20%. The release sustained till day 20 and reached a plateau corresponding to ˜30% of the loaded NGF.

TABLE 2
Loading efficiency determined by radioactivity measurement

| | AVE (%) | STDEV (%) |
|---|---|---|
| 100 ng FGF-2 | 68.87 | 0.15 |
| 500 ng FGF-2 | 67.68 | 0.30 |
| 100 ng NGF | 60.64 | 1.08 |
| 500 ng NGF | 53.63 | 0.15 |

In addition to the method above, enzyme-linked immunosorbant assay (ELISA) is also a common method used to examine the loading efficiency. Here, after PEAD/heparin/FGF-2 formation, this solution was coated onto the plate for overnight. An anti-FGF-2 polyclonal antibody was later applied for detection. The result (Table 3) shows when FGF-2 was added into PEAD/heparin complex, less than 99% of FGF-2 can be detected by the antibody. For NGF, a sandwich ELISA was applied for the experiment. A NGF-specific monoclonal antibody was coated on the plate first followed by PEAD/heparin/NGF incubation. Another anti-NGF antibody was then added for detection. Similar as the result of FGF-2, less than 98% of NGF can be detected by ELISA. Combined the results of radioactivity, we appreciate when either FGF-2 or NGF was loaded into PEAD/heparin complex; more than half percent of FGF-2 or NGF would be precipitated down and formed the pellet after centrifugation. However even for the growth factor not precipitated, it should not maintain in a free form but bind to heparin or PEAD/heparin. Consequently, it cannot be recognized by the antibody.

TABLE 3
Loading efficiency determined by ELISA

| | Ave (%) | STDEV (%) |
|---|---|---|
| 100 ng FGF-2 | 99.99 | 2.8E−05 |
| 100 ng NGF | 99.98 | 0.0038 |

Once FGF-2 or NGF was loaded into PEAD/heparin complex, the bulk solution was filled, collected and refilled at different time points. The radioactivity of the collected solution was used to calculate the amount of growth factor released from PEAD/heparin and generate the release profile. For FGF-2, the result (FIG. 4A) shows an initial burst of ˜10% of release after the first day. Thereafter, the release was close to linear and sustained for six weeks. After six weeks, PEAD/heparin still contained ˜30% of FGF-2. For the higher dosage of FGF-2, the release was slower, but there is no huge difference. The other growth factor, NGF, the release (FIG. 4B) is clearly faster. The initial burst reached almost 20%. The release sustained till day 20 and reached a plateau which contained ˜30% of loaded NGF.

The loading efficiency and release profile indicated PEAD/heparin complex has good affinity toward the growth factors. To support this system can be applied for growth factor delivery furthermore, we tested the bioactivity of the growth factor released from the complex. For FGF-2 bioactivity, the proliferation of human aortic endothelial cells (HAECs) was compared between the control which is no supplemental growth factors, bolus FGF-2, PEAD/heparin-complexed FGF-2 and endothelial cell (EC) culture supplement which contained low concentrations of epithelial growth factor (EGF), FGF-2, insulin-like growth factor-1 (IGF-1), vascular endothelial growth factor (VEGF) and hydrocortisone. In this experiment HAECs were cultured on the lower chamber and different conditions of FGF-2 or supplement was added in a cell culture insert. If FGF-2 released from PEAD/heparin complex was still bioactive, it would pass through the pore of the insert to promote HAEC proliferation. The result (FIG. 5) which was 4 days' culture shows both bolus FGF-2 and complexed FGF-2 had higher proliferation than the control and EC culture supplement. The proliferation of complexed FGF-2 was 2.69 fold of that of control and 1.26 fold of that of EC culture supplement. Of note, there is no statistical difference between bolus FGF-2 and PEAD/heparin-complexed FGF-2.

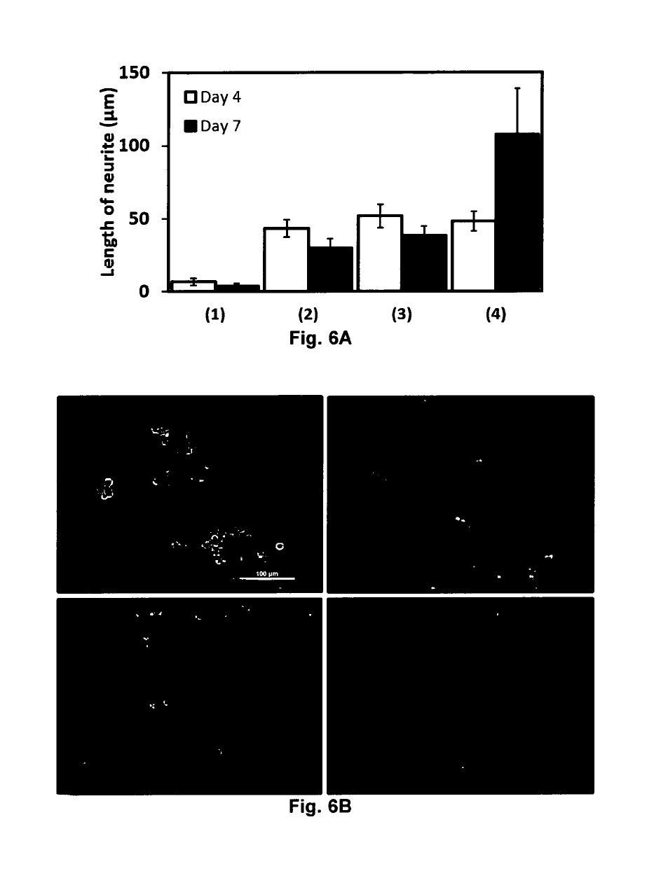

For the bioactivity of NGF, a common cell line, PC-12 cells, was chosen as a model in this study. With the stimulation of NGF, PC-12 cells would start differentiation and grow neurites. Therefore, the lengths of neurites were utilized as an index for determining the bioactivity. The study included four groups, control which was no NGF, bolus NGF, heparin-stabilized NGF and PEAD/heparin-complexed NGF. The result (FIG. 6A) shows after 4 days culture, all three experimental groups had significant longer neurites than the control group. Among them, heparin-stabilized NGF had the longest neurite length. Complexed NGF had the neurite length between bolus and heparin-stabilized NGF but shows no statistical difference with those two. Continue observing till one week, we found even prominent differences between each group (FIGS. 6A and 6B). The control group still had the shortest neurite length. However for bolus NGF and heparin-stabilized NGF, the neurite lengths all became shorter compared with 4 days' culture. PEAD/heparin-complexed NGF, on the contrary, continued stimulating the neurite growth and reached over double length of 4 days' culture (48 μm vs. 108 μm). This result reveals PEAD/heparin-complexed NGF has higher bioactivity for long term culture.

### Example 2—Enhancement of Angiogenesis and Vascular Maturity Via an Injectable Delivery Matrix

### Materials and Methods

### Synthesis of PEAD—

PEAD was synthesized essentially as described in Example 1. The chemical structure was characterized using an UltraShield Plus 600 NMR (Bruker BioSpin) and a Nicolet iS10 spectrometer (Thermo Fisher Scientific).

### Delivery Vehicle Preparation and Scanning Electron Microscopy—

For preparation of the delivery vehicle, PEAD dissolved in the deionized water was mixed with heparin solution under the stirring condition. The complex was dropped on an aluminum stub, lyophilized, sputtered with gold, and the morphology was viewed with a Jeol 9335 field emission gun SEM (Jeol).

### Western Blotting—

Both PEAD and heparin were dissolve in normal saline (0.9% NaCl_{(aq)}) to prepare the concentration of 10 mg/ml. For preparation of the delivery matrix, 500 ng of FGF-2 was first mixed with 10 μl of heparin solution and then 50 μl of PEAD solution. 20× of normal saline and deionized water were used to adjust the desired ionic strength to obtain the delivery vehicle in 1×, 2× and 5× of saline. The delivery matrix was equilibrated at room temperature for 15 min followed by the centrifugation at 12,100 g for 10 min. The supernatant was collected, mixed with the common sample buffer and denatured at 95° C. for 5 min. 15% of sodium dodecyl sulfate polyacrylamide gel electrophoresis (SDS-PAGE) was utilized for separation followed by protein blotting on a polyvinylidene fluoride (PVDF) membrane. A rabbit anti-human FGF-2 polyclonal antibody (Peprotech) was applied for recognition followed by a secondary horse peroxidase conjugated anti-rabbit IgG antibody (Sigma).

### Tube Formation of HUVECs in Fibrin Gel—

HUVECs (Lonza) were maintained in EGM-2 basal medium supplemented with growth factors according to the instruction. For tube formation, 8×10⁴ cells (passage 7) were mixed with fibrinogen solution containing FGF-2 (100 ng) or the same amount of FGF-2 in the delivery matrix. After addition of thrombin, the whole solution was gelled at 37° C. for 30 min. The gel was last overlaid with 600 d of the basal medium to provide the basic nutrient. After incubation for 3 days, the phase contrast images were taken by an inverted microscope Eclipse Ti (Nikon).

### Animal Care and Subcutaneous Injection—

Under isoflurane anesthesia, 65 μl of saline, the delivery vehicle (500 μg of PEAD and 100 μg of heparin), the bolus FGF-2 (500 ng of FGF-2) or the delivery matrix (500 μg of PEAD, 100 μg of heparin and 500 ng of FGF-2) was subcutaneously injected in the left back of Male Balb/cJ mice with an average age of 6-7 weeks. The right back which did not receive injection was later utilized as the contralateral site. All groups contained 4 to 8 mice.

### Hemoglobin Quantification—

The animals were sacrificed at post-injection week 1, 2 and 4. The subcutaneous tissue having a dimension of 1.5 cm×1.5 cm was harvested at the injection site and the contralateral site. The hemoglobin in the harvested tissue was extracted by addition of in 500 μl of the hemolysis buffer containing 17 mM of Tris-HCl (pH 7.4) and 0.75 wt % ammonium chloride and incubation for 24 h. The absorbance at 410 nm corresponding to the hemoglobin Soret band was recorded by a SynergyMX plate reader (Biotek). All values were normalized to the one of the saline injection.

### Immunofluorescent Analysis—

The harvested subcutaneous tissue was embedded and frozen in Tissue-Tek OCT compound (Sakura Finetek USA). Sections of 5 nm thickness were cut with a cryo-microtome and stored at −80° C. For staining CD31-positive cells, a rat anti-mouse CD31 monoclonal antibody (BD Biosciences) was applied first followed by a Cy3-conjugated anti-rat IgG polyclonal antibody. For staining alpha-SMA-positive cells, a FITC-conjugated anti-SMA monoclonal antibody (Sigma) was utilized according to the provided instruction. For vWF-positive cells, a FITC-conjugated anti-vWF antibody was used for staining. All slides were last counterstained with DAPI (Invitrogen). The fluorescent images were taken using a Fluoview 1000 Confocal microscope (Olympus).

### Quantification of CD31- and Alpha-SMA-Positive Cells—

For the 4-week slides, random fields were chosen for comparison between each condition. The number of CD31- or alpha-SMA-positive cells were counted and confirmed by DAPI-positive nuclei. The value was divided by the area of the tissue and normalized to that of the control group.

### Statistical Analysis—

ANOVA followed by post-hoc Bonferroni test was utilized to compare the number of CD31- and alpha-SMA-positive cells between all conditions. Data is presented as mean±standard deviations. *p value <0.05; **p value <0.01.

### Results

### Interaction Between [PEAD:Heparin] and FGF-2—

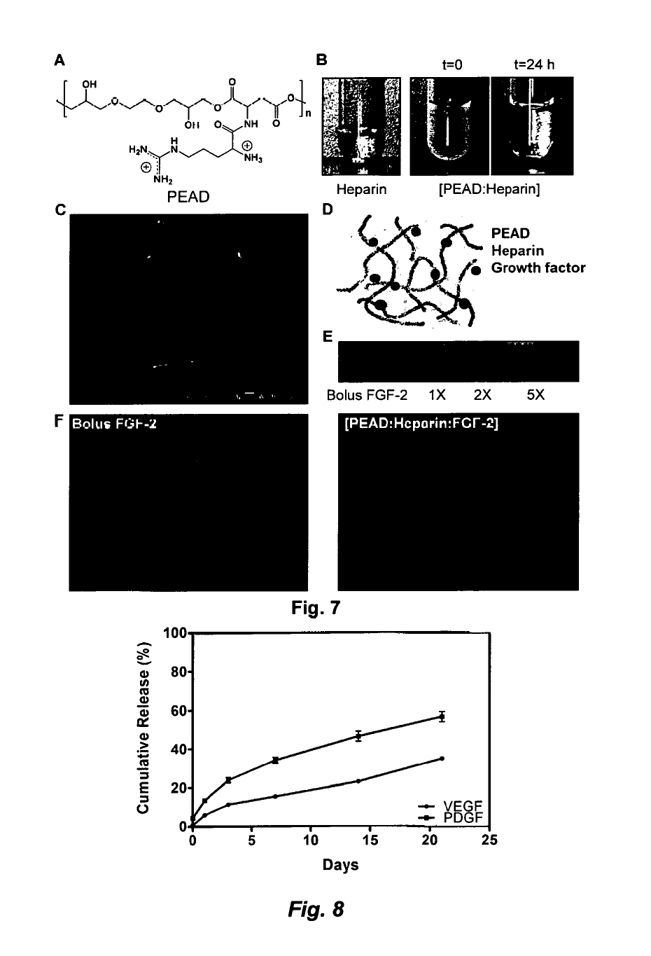

Polycations have many biological applications but their low biocompatibility may compromise the clinical potentials. Solving this problem, we have synthesized new generation of polycations. We propose that the polycations composed of natural building blocks and connected by hydrolysable linkage can have better biocompatibility. One biocompatible polycation, poly(ethylene argininylaspartate diglyceride) (PEAD), is composed of aspartyl, arginyl and diglyceride moieties (FIG. 7(A)). The repeating unit of PEAD is linked by hydrolysable ester bonds. PEAD has advantages including the superior biocompatibility examined by in vitro and in vivo tests. The amino and guanidine groups which are protonated under the physiological condition render PEAD the ability to interact strongly with natural polyanions, such as heparin and hyaluronic acid. Macroscopic observation found that PEAD lowered the solubility of heparin in the aqueous solution by forming [PEAD:Heparin] complex through charge interaction (FIG. 7(B)). [PEAD:Heparin] complex continued aggregation and sedimented to the bottom of the test tube. The speed of sediment depended on the concentration of the solution and also the mass ratio of individual polymer. Generally, a coacervate was completely formed after 24 h incubation. The coacervate can be resuspended easily and reversed to the turbid state. The microscopic picture revealed that the morphology of the [PEAD:Heparin] complex was mainly composed of globular and fibrous domains (FIG. 7(C)). The diameters of the domains covered a range from μm to sub-μm. Our prior study examined that the morphology of [PEAD:Heparin] complex was dependent on the mass ratio individual polymer. The globular domains were contributed by heparin whereas the fibrous ones were from PEAD.

Due to the high affinity of heparin to many growth factors, we propose the mechanism that [PEAD:heparin] complex is able to incorporate high amounts of growth factors through their heparin-binding domains (FIG. 7(D)). With the increase of the ionic strength of the solution, the binding between PEAD, heparin and heparin-binding growth factors is reduced and therefore the growth factors would released from the complex. This hypothesis was later confirmed by the loading experiment of FGF-2 (FIG. 7(E)). Under normal saline condition (0.9% NaCl_{(aq)}), FGF-2 (500 ng) was completely precipitated by [PEAD:Heparin] complex. While increasing the ionic strength to 2 folds of the normal saline did not change the result, 5 folds of the normal saline broke the charge interaction between the polymers and FGF-2. Therefore, [PEAD:Heparin] complex was no longer able to precipitate FGF-2 at this condition.

Comparing the bioactivity of the bolus and the delivery matrix, we applied the established method trapping human umbilical vein endothelial cells (HUVECs) together with the bolus FGF-2 or the delivery matrix. The culture medium was overlaid on the gel to provide necessary nutrients and allow materials exchange. After three days, we observed significant differences between the bolus FGF-2 and the delivery matrix groups (FIG. 7(F)). The bolus FGF-2 induced very less degree of differentiation of HUVECs. Most cells were rounded and scattered in the gel. On the other hand, in the delivery matrix group, HUVECs completely differentiated and aligned to develop a complex mesh. The result strongly indicated the higher bioactivity of the delivery matrix. We expected the possible mechanism that FGF-2 which had high affinity with heparin was confined in the gel by the [PEAD:heparin] complex. Some of the bolus FGF-2 in the gel, however, had diffused to the overlaid medium and was not able to stimulate cells efficiently.

[PEAD:Heparin:FGF-2] Promotes Higher Angiogenesis than Bolus FGF-2—

To examine the in vive ability of the delivery matrix, the delivery matrix containing 500 ng of FGF-2 was subcutaneously injected in the back of male BALB/cJ mice. The angiogenic effect was compared between the control, the delivery vehicle and 500 ng of FGF-2. Macroscopic observation of the tissue found extensive formation of blood vessels at the injection site whereas the contralateral showed no obvious effect. This indicates the efficacy of the delivery matrix to promote angiogenesis and also localize the distribution of FGF-2. Quantitative comparison of the concentration of hemoglobin at three time points revealed that the delivery matrix group had higher amounts of hemoglobin from 2 weeks post-injection. On the other hand, the bolus FGF-2 did not have statistical difference with the control and the delivery vehicle groups. After 4 weeks, the delivery matrix group still yielded a significantly higher amount of hemoglobin than other three groups. Possibly it reflected the long term stability of blood vessels or the long term bioactivity of the delivery matrix. Further comparing the ratio of hemoglobin at the injection site and the contralateral site, we found the ratios were significantly larger than 1 after 2 weeks. It correlated with the macroscopic observation that the angiogenic activity of the delivery matrix was confined at the injection site. The bolus group, however, had the ratio close to 1 for every time point and was significantly lower than the delivery matrix after 2 weeks.

Hematoxylin and eosin staining revealed the gross appearance of the delivery vehicle and the control groups having similar feature suggesting the delivery vehicle had no angiogenic effect. For the bolus FGF-2, we observed cells aggregated together in the hypodermis region. Muscle fibers were also found together with aggregated cells. Yet, clear blood vessel feature was rarely seen in all tissue sections. On the contrary, the delivery matrix showed significant blood vessel formation. Circular and closed pattern of nucleated cells surrounded by the muscle bundles were clearly observed. The lumen of vessel was filled with red cells further supporting the function of the nascent blood vessel.

### [PEAD:Heparin:FGF-2] Stimulates Proliferation of Endothelial Cells and Mural Cells—

We later studied the extent of angiogenesis by immunofluorescent analysis. Two specific markers, CD31 and α-smooth muscle actin (alpha-SMA), were stained for the angiogenic effect and maturation of blood vessels induced by the delivery matrix. After 1 week, both the delivery matrix and bolus FGF-2 induced more CD31-positive cells than the control. This can be explained by the proliferation of endothelial cells stimulated by FGF-2. On the other hand very low numbers of alpha-SMA-positive cells were observed for all groups. After 2 weeks, higher amounts of endothelial cells can still be found in the delivery matrix and bolus FGF-2 whereas only the delivery matrix induced a significant amount of alpha-SMA-positive cells. In addition, the blood vessels were also well organized as the circular features of endothelial cells lined by the mural cells. These features became more significant after 4 weeks revealed by the number of blood vessels in the field. Compared to the delivery matrix, other three groups had similar features that most endothelial cells were naked without the surrounding of mural cells. The higher magnified micrograph further confirmed the complete structure of blood vessels having a distinctive alignment of endothelial cells and mural cells.

### [PEAD:Heparin:FGF-2] Induced Higher Amounts of CD31- and Alpha-SMA-Positive Cells—

To get statistical comparison, random fields were chosen for quantifying the number of endothelial cells and mural cells. The result suggested that the delivery matrix increased 72% of the number of CD-31 positive cells of the control, 69% of the number of the delivery vehicle and 41% of that of the bolus FGF-2. All the comparisons were statistically significant with p values lower than 0.01. On the other hand, although the average number of the bolus group was higher, there was not statistical difference with the control and the delivery vehicle groups. Consistent with the qualitative observation, the delivery matrix induced more proliferation of the endothelial cells. More striking difference was the number of alpha-SMA-positive cells. Very few alpha-SMA-positive were found in the field beside the delivery matrix. The quantitative result also pointed that the delivery matrix group was 3.81 folds of that of the control, 3.15 folds of the delivery vehicle and 2.82 folds of the bolus FGF-2. All comparison had p values lower than 0.01. Again no statistical difference was revealed among other three groups. Collectively, both angiogenic markers strongly supported the higher extent of blood vessel formation stimulated by the delivery matrix.

### [PEAD:Heparin:FGF-2] Supports Maturity of Nascent Blood Vessels—

Further examining the maturity of the newly formed blood vessels induced by the delivery matrix, we stained a series of blood vessel associated markers. Desmin, a component of intermediate filament, is a commonly used marker for mural cells. We observed desmin co-expressed in alpha-SMA-positive blood vessels. Additionally, alpha-SMA-negative but desmin-positive blood vessels were also found in the field. It possibly reflected the heterogeneity of pericytes which had low alpha-SMA expression at capillaries. Von Willebrand factor (vWF) being an important molecule participating in hemostasis was stained to prove the thrombotic ability of the nascent blood vessels. We found that the delivery matrix induced rich expression of vWF. The evident overlap of CD31 and vWF signals confirmed the nascent endothelial cells were fully functional. Last, smooth muscle myosin heavy chain (SMMHC) representing the contractibility of smooth muscle cells was co-stained with alpha-SMA to evaluate the functionality of nascent blood vessels. The result indicated the blood vessels induced bolus FGF-2 were smaller and did not have obvious expression of SMMHC even with the ones having abundant expression of alpha-SMA. For the delivery matrix, smaller blood vessels did not express SMMHC as was the case of bolus FGF-2, but more importantly, it also contained bigger blood vessels having significant overlap of alpha-SMA and SMMHC.

### [PEAD:Heparin:FGF-2] Promote Stabilization of Endothelial Cells by Pericytes at Early Stage—

The above results support that for the long term the delivery matrix is able to enhance the maturity of the nascent blood vessels especially by increasing the number of smooth muscle cells and enhancing their functions. To investigate its effect to pericytes which are important mediators involving in the early stage of the angigenic process, CD31 was co-stained with the pericyte specific marker, platelet derived growth factor 3 (PDGFR β). We observed after 1 week many CD31-positive endothelial cells clustered with PDGFR β-positive cells in the delivery matrix group whereas no overlap was seen in the bolus FGF-2 group. The association supposedly indicated the interaction between pericytes and endothelial cells.

### Example 3

During angiogenesis, vascular endothelial growth factor (VEGF) is required early to initiate neovessel formation while platelet-derived growth factor (PDGF-BB) is needed later to stabilize the neovessels. The spatiotemporal delivery of multiple bioactive growth factors involved in angiogenesis, in a close mimic to physiological cues, holds great potential to treat ischemic diseases. To achieve sequential release of VEGF and PDGF, VEGF was embedded in a fibrin gel and PDGF in a heparin-based coacervate that is distributed in the same fibrin gel. In vitro, we show the benefits of this controlled delivery approach on cell proliferation, chemotaxis, and capillary formation. A rat myocardial infarction (MI) model demonstrated the effectiveness of this delivery system in improving cardiac function, ventricular wall thickness, angiogenesis, cardiac muscle survival, and reducing fibrosis and inflammation in the infarct zone compared to saline, empty vehicle, and free growth factors. Collectively, our results show that this delivery approach mitigated the injury caused by MI and may serve as a new therapy to treat ischemic hearts pending further examination. A controlled delivery system was prepared to control the spatiotemporal cues and protect the bioactivity of VEGF and PDGF. The controlled delivery system comprises fibrin gel and a biocompatible heparin-based coacervate. Fibrin gel, formed through the polymerization of fibrinogen by thrombin, is commercially available. Complex coacervates are formed by mixing oppositely charged polyelectrolytes resulting in spherical droplets of organic molecules held together noncovalently and apart from the surrounding liquid and have shown potential in sustained protein delivery. VEGF was embedded into the fibrin gel, while PDGF was loaded into the coacervate then embedded into the gel. The coacervate was used to control the release of PDGF based on its affinity to heparin. This system provided rapid release of VEGF followed by slow and sustained release of PDGF from a single injection. Here we report the effects of sequentially delivered VEGF and PDGF on revascularization and heart function after MI in rats.

### Materials and Methods

### Release Kinetics Assay

The release assay (n=3) was performed using 100 ng of VEGF₁₆₅ and 100 ng of PDGF-BB (PeproTech, Rocky Hill, N.J.). PDGF Coacervate was made by mixing PDGF with heparin first (Scientific Protein Labs, Waunakee, Wis.), then with the polycation, poly(ethylene arginyl aspartate diglyceride) (PEAD) at PEAD:heparin:GF mass ratio of 50:10:1. Fibrin gel was made by mixing 90 μl of 20 mg/ml fibrinogen solution (Sigma-Aldrich, St. Louis, Mo.) containing unbound VEGF and the PDGF coacervate with 5 μl of 1 mg/ml thrombin solution (Sigma-Aldrich, St. Louis, Mo.) and 5 μl of 1 mg/ml aprotonin solution (Sigma-Aldrich, St. Louis, Mo.). A 100 μl of 0.9% saline was deposited on top of fibrin gel to be collected at 1 hr, 16 hrs, 1, 4, 7, 14, and 21 days. The samples were incubated at 37° C. After centrifugation at 12,100 g for 10 min, supernatant was aspirated and stored at −80° C. to detect amount of released GFs by ELISA kits (PeproTech, Rocky Hill, N.J.). The absorbance at 450/540 nm was measured by a SynergyMX plate reader (Biotek, Winooski, Vt.). Normalizing standards (n=3) were prepared using the same amounts of free GFs in 100 μl of 0.9% saline.

### Smooth Muscle Cell Chemotaxis Assay

Chemotactic media was prepared as 500 μl MCDB-131+10% fetal bovine serum (FBS) per well in a 24-well plate with group-specific addition of saline (basal media), empty vehicle, or 100 ng free PDGF or in the coacervate. 8 μm pore size culture inserts (BD Falcon, Franklin Lakes, N.J.) were placed in each well and 10⁴ baboon SMCs were pipetted into the insert in 200 μl basal media and plate was incubated at 37° C. After 12 hrs, cells remaining insidelte insert were removed from the upper surface of the membrane with a cotton swab. Cells that had migrated to the lower surface of the membrane were then fixed in methanol for 15 min. Cells were incubated for 15 min in the dark with PicoGreen fluorescent dye from Quant-iT PicoGreen dsDNA Kit (Molecular Probes, Eugene, Oreg.), diluted 200-fold to working concentration in DPBS. Cells were imaged with a fluorescent microscope (Eclipse Ti; Nikon, Tokyo, Japan) and images were taken in the center of each well in three wells per group and counted manually.

### Endothelial and Smooth Muscle Cells Proliferation Assays

Human umbilical vein endothelial cells (HUVEC) (ATCC, Manassas, Va.) or baboon SMCs were seeded at 10⁴ cells per well in a 96-well plate and cultured in EGM-2 media (Lonza, Walkersville, Md.) and MCDB131+0.2% FBS media, respectively. Group-specific additions were made to media with GF concentrations at 20 ng/ml per well for SMCs and 25 ng/ml of each GF per well for HUVEC. The plates were incubated for 48 hrs at 37° C. 20 μl of preprepared BrdU label was then added for 4 hrs and the proliferation assays were performed according to kit's instructions (Millipore, Temecula, Calif.). The absorbance at 450/540 nm was measured by a SynergyMX plate reader. Absorbance proliferation values were normalized to basal media value.

### Ex Vivo Rat Aortic Ring Assay

Thoracic rat aortae (n=3 per group) were dissected according to established protocols (A.C. Aplin, et al. The aortic ring model of angiogenesis, Methods Enzymol 443 (2008) 119-136 and R. S. Go, et al., The rat aortic ring assay for in vitro study of angiogenesis, Methods Mol Med 85 (2003) 59-64), cleaned from fibro-adipose tissue, and cut into approximately 1.5 mm ring segments. Rings were serum-starved overnight in serum-free endothelial basal medium (EBM). Next day, the rings were embedded in the center of a 3D fibrin matrix that contained different treatment groups (GF dose of 250 ng) with luminal axis perpendicular to the bottom of the well in a 24-well plate. 500 μL of EBM was placed on top of gel. Rings were incubated at 37° C. for 6 days. Rings were then imaged using brightfield (BF) microscopy and quantified in terms of microvasculature sprouting area in 3 wells per group.

### Rat Acute Myocardial Infarction Model

MI and injections were performed as previously described (S. Dobner, et al., A synthetic non-degradable polyethylene glycol hydrogel retards adverse post-infarct left ventricular remodeling, J Card Fail 15 (2009) 629-636). Briefly, 6-7 week old male Sprague-Dawley rats (Charles River Labs, Wilmington, Mass.) were anesthetized with isoflurane (Butler Schein, Dublin, Ohio), intubated, and connected to a mechanical ventilator. The ventral side was shaved and a small incision was made through the skin. The muscle and ribs above heart were separated. The heart was exposed and MI was induced by ligation of the left anterior descending (LAD) coronary artery using a 6-0 polypropylene suture (Ethicon, Bridgewater, N.J.). Five minutes after the induction of MI, 100 μl of saline, empty vehicle, free VEGF+PDGF (1.5 μg of each GF), or sequentially delivered VEGF+PDGF (using fibrin gel-coacervate system) solutions were injected intramyocardially at 3 equidistant points around the infarct zone using a 31 G needle (BD, Franklin Lakes, N.J.). For injections of fibrin gel, thrombin was added to fibrinogen solution and injected shortly before gelation. The chest was closed and the rat was allowed to recover. After 4 weeks, all animals were sacrificed and hearts were harvested for histological and immunohistochemical evaluation.

### Echocardiography

Echocardiography was performed 2 days before surgery (baseline) and at 2, 14, and 28 days post-MI surgery to evaluate cardiac function. Short-axis videos of the left ventricle (LV) by B-mode were obtained using a Vevo 770 high-resolution in vive micro-Imaging system (Visual Sonics, Ontario, Canada). End-systolic area (ESA) and end-diastolic area (EDA) were measured using NIH ImageJ 1.46r and fractional area change (FAC) was calculated as: [(EDA−ESA)/EDA]*100%. Percent improvements of one group over another were calculated as the difference between the % drops in FAC values of the first and second groups divided by the higher % drop of the two groups.

### Histological Analysis

At 4 weeks post-infarction, rats were sacrificed by injecting 2 ml of deionized (Dl) water saturated with potassium chloride (KCl) (Sigma Aldrich, St. Louis, Mo.) in the LV to arrest the heart in diastole. Hearts were harvested and frozen in OCT compound. Specimens were sectioned at 6 mm thickness from apex to the ligation level with 500 μm intervals. Sections were fixed in 2-4% paraformaldehyde (fisher Scientific, Fair Lawn, N.J.) prior to all staining procedures.

Hematoxylin and eosin (H&E) staining was performed for general evaluation. Five to six H&E stained slides from each group were randomly selected and the ventricular wall thickness in the infarct zone of each was measured near the mid-section level of the infarct tissue using NIS Elements AR imaging software (Nikon Instruments, Melville, N.Y.).

For assessment of fibrosis, picosirius red staining was used to stain collagen fibers. The fraction area of collagen deposition in the infarct region was measured by NIS Elements AR software. Five to six slides from each group were used for quantification near the mid-section level of the infarct tissue.

### Immunohistochemical Analysis

For evaluation of inflammation, a mouse anti-rat CD68 (Millipore, Temecula Calif.) was used followed by an Alexa fluor 594 goat anti-mouse antibody (Invitrogen, Carlsbad, Calif.). For evaluation of angiogenesis, ECs were detected by a rabbit anti-rat Von Willebrand factor (vWF) antibody (US Abcam, Cambridge, Mass.) followed by an Alexa fluor 594 goat anti-rabbit antibody (Invitrogen Carlsbad, Calif.). Mural cells were detected by a FITC-conjugated anti-α-smooth muscle actin (α-SMA) monoclonal antibody (Sigma Aldrich, St. Louis, Mo.). Viable cardiomyocytes were detected by staining using a mouse anti-rat cardiac troponin I (cTnI) antibody (US Abcam, Cambridge, Mass.) followed by an Alexa fluor 488 goat anti-mouse antibody (Invitrogen, Carlsbad, Calif.). All slides were last counterstained with DAPI (Invitrogen, Carlsbad, Calif.).

For quantification, four to five slides from each group were utilized near the mid-section level of the infarct tissue. The numbers of CD68-positive cells and vWF- and α-SMA-positive vessels were counted and reported per mm² areas. The cTnI-positive fraction area in the infarct region was measured by NIS Elements AR software. Intensity of fluorescence was determined by ImageJ and normalized to the background value.

### Statistical Analysis

Results are presented as means±standard deviations (SD). GraphPad Prism 5.0 statistical software (La Jolla, Calif.) was used for statistical analysis. One-way ANOVA followed by Tukey's HSD test was used for in vitro assays, histological and immunohistochemical analyses. Two-way ANOVA followed by Bonferroni post-hoc test was used for echocardiography analysis. P value <0.05 was considered significantly different.

### Results

### Fibrin Gel-Coacervate System Achieves Sequential Delivery

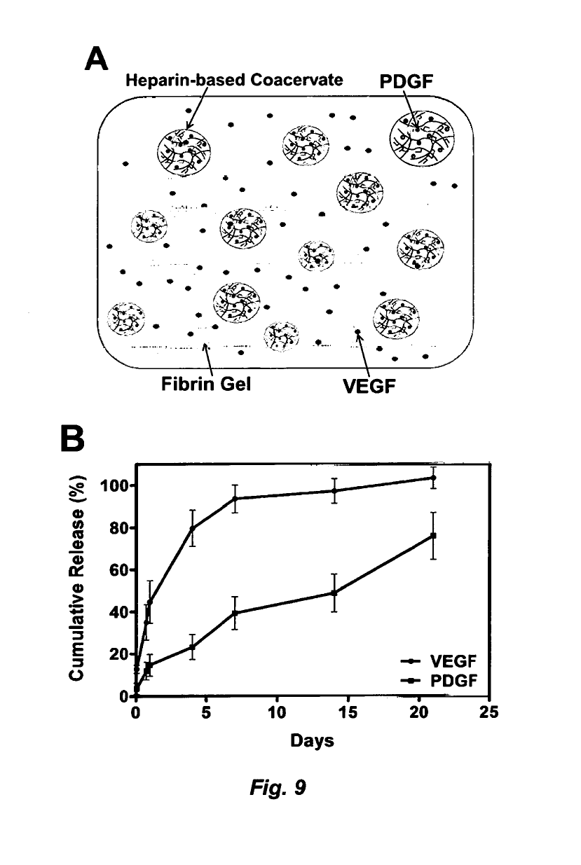

Previously, we studied VEGF release from the coacervate which was relatively slow because of its mid-range affinity for heparin (k_{d}=165 nM) (H. K. Awada, et al., Dual delivery of vascular endothelial growth factor and hepatocyte growth factor coacervate displays strong angiogenic effects, Macromol Biosci 14 (2014) 679-686 and S. Ashikari-Hada, et al., Characterization of growth factor-binding structures in heparin/heparan sulfate using an octasaccharide library, J Biol Chem 279 (2004) 12346-12354). With a weaker heparin-binding affinity (k_{d}=752 nM) (I. Freeman, et al., The effect of sulfation of alginate hydrogels on the specific binding and controlled release of heparin-binding proteins, Biomaterials 29 (2008) 3260-3268), PDGF release from the coacervate occurs faster than for VEGF (FIG. 8). A proper therapeutic angiogenesis process needs a sequential release of VEGF first followed by PDGF. In order to obtain faster VEGF release, we embedded it in a fibrin gel without loading it into the coacervate. We then loaded PDGF in the coacervate to provide its sustained release and embedded it in the same fibrin gel (FIG. 9(A)). The loading efficiencies were 87% for VEGF and 97% for PDGF as observed 1 hour after loading. VEGF had a burst release of 44% including the unloaded amount by day 1, while PDGF had a minimal burst release of 14% (FIG. 9(B)). Having a significant release of VEGF by day 1 might prove beneficial for angiogenesis and heart function after MI (L. Zangi, et al., Modified mRNA directs the fate of heart progenitor cells and induces vascular regeneration after myocardial infarction, Nat Biotechnol 31 (2013) 898-907). This delivery system achieved sequential release kinetics, where 95% of VEGF was released by one week and only 40% of PDGF, which continued to release up to 75% after three weeks (FIG. 9(B)). The in vivo release rate can be further influenced by fibrinolysis, hydrolytic degradation of the PEAD polycation, enzymatic degradation by esterases and heparinases, and dissociation of the coacervate in an ionic environment. Thus, in vivo release is expected to be faster. Overall, the release kinetics attained with the fibrin gel-coacervate delivery vehicle may enhance the formation of neovasculature based on the physiological roles of VEGF and PDGF during angiogenesis.

### PDGF Coacervate Induces SMC Chemotaxis and Proliferation

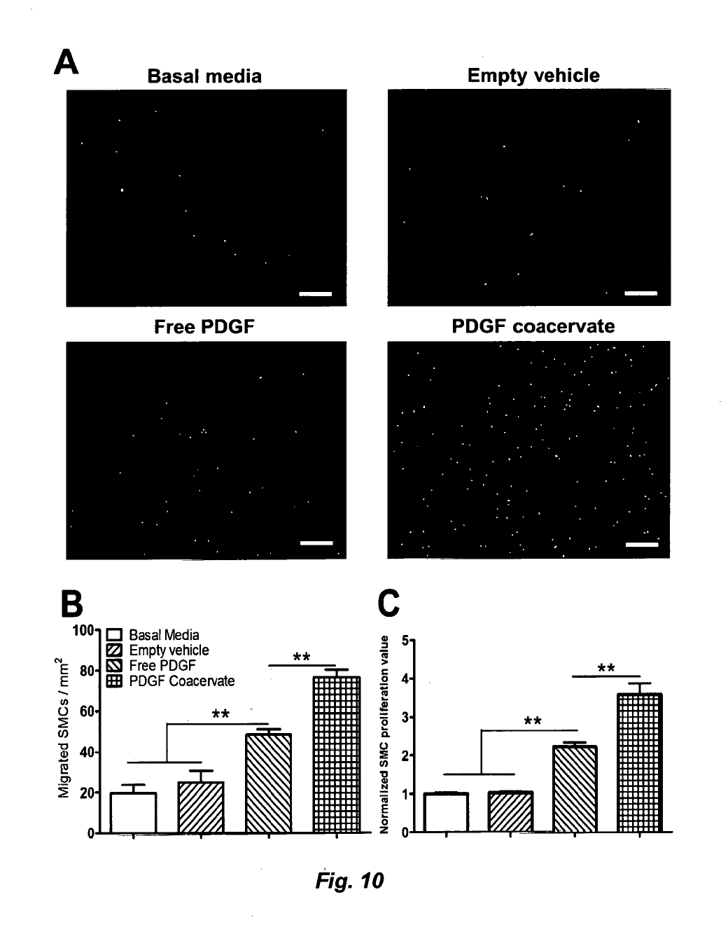

Here, we evaluated the effect of PDGF released from the coacervate on SMC migration using a Boyden chamber assay. Free PDGF induced significantly more SMC migration compared to controls, however the same dose of PDGF delivered by the coacervate had the greatest chemotactic effect compared to all groups (FIG. 10(A,B)). The empty vehicle was also demonstrated to be inert with no effect on cell migration compared to basal media alone.

We also tested the effect of coacervate-released PDGF on SMC proliferation using a BrdU assay. Again, we observed no significant effect of the vehicle compared to basal media control. Both free PDGF and PDGF coacervate induced significant SMC proliferation compared to control groups. However, PDGF coacervate also increased cell proliferation compared to free PDGF (FIG. 10(C)). Collectively, these results demonstrate that PDGF released from the PEAD coacervate is highly bioactive and can stimulate proliferation and migration of SMCs in vitro.

### Sequential Delivery Improves EC Proliferation and Vessel Sprouting

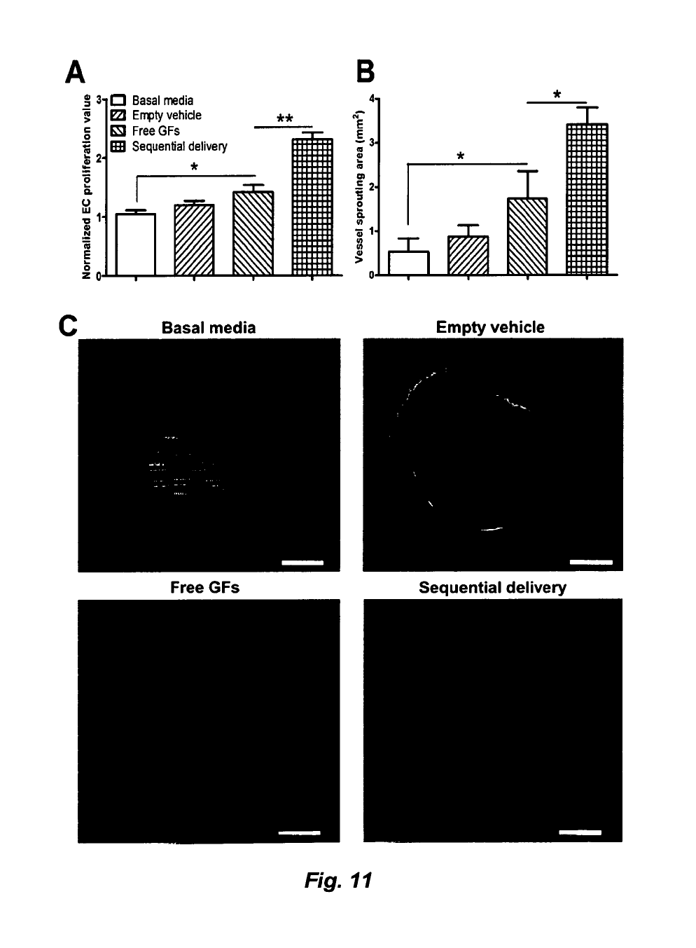

In order to evaluate the potential benefit of sequential release of VEGF and PDGF, we performed EC proliferation and aortic ring vessel sprouting assays. We hypothesized that high initial PDGF concentrations would reduce the effect of VEGF on ECs. Free VEGF+PDGF induced significantly more proliferation than basal media, but not more than empty vehicle, which showed no difference compared to basal media. However, sequentially delivered VEGF+PDGF induced significantly more proliferation than both control and free GFs (FIG. 11(A)).

In the aortic ring assay, free GFs induced significantly more microvessel outgrowth and longer sprouts from ring segments compared to basal media but not compared to empty vehicle. In contrast, sequential delivery showed significantly larger sprouting area than all groups (FIG. 11(B,C)). Taken together, these experiments suggest that PDGF has an antagonistic effect on VEGF-mediated angiogenic responses in vitro which can be avoided by a sequential delivery approach.

### Sequential Delivery of VEGF and PDGF Improves Overall Cardiac Function

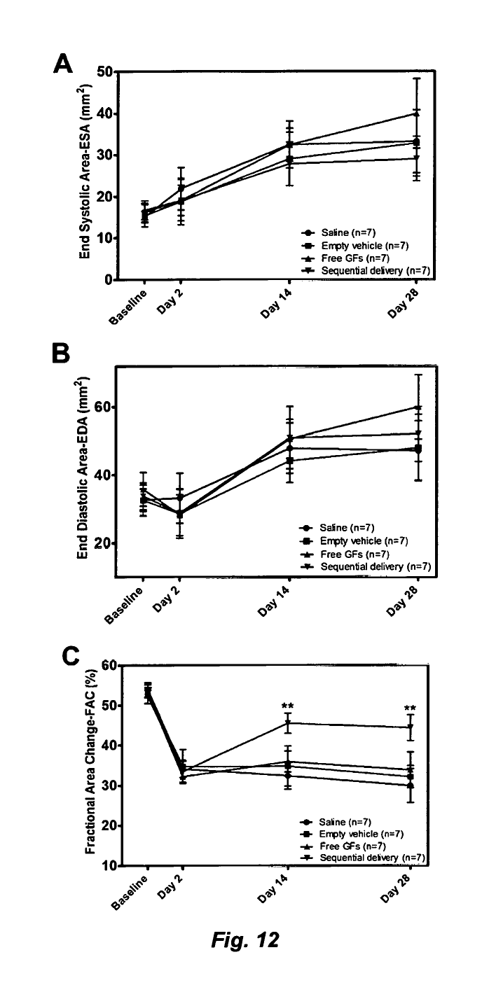

We next evaluated the in vivo effect of sequential delivery in a rat MI model comparing saline, empty vehicle, free VEGF+PDGF, and sequentially delivered VEGF+PDGF. We evaluated changes in LV contractility using 2-D echocardiography and reported heart function as fractional area change (FAC). ESA and EDA values were similar for all groups suggesting little to no effect on ventricular dilation over the time period evaluated (FIG. 12(A,B)).

MI induction was confirmed by a significant drop in FAC 2 days after infarction (FIG. 12(C)). No significant differences were found between groups at baseline or at day 2. At 2 weeks, sequential delivery group showed a significant improvement in cardiac function compared to all other groups. FAC values were 32.5±3.3% for saline, 34.9±5% for empty vehicle, 36±2.6% for free GFs, and 45.6±2.5% for sequential delivery (FIG. 12(C)). No significant differences were found between saline, empty vehicle, and free GFs values at 2 weeks. This result represented a 60% improvement by sequential delivery over free GFs and 68% over saline.

At 4 weeks, FAC declined slightly for all groups, but sequential delivery group maintained its improvement in cardiac function with a significantly higher FAC compared to all groups. FAC values were 30±4.3% for saline, 32.2±2.8% for empty vehicle, 33.9±4.4% for free GFs groups, and 44.4±3.2% for sequential delivery (FIG. 12(C)). The sequential delivery value represented a 59% improvement over free GFs and 66% over saline. The ability of sequential delivery to improve and maintain the cardiac function 4 weeks after MI stresses the importance of spatiotemporal presentation towards the effectiveness of VEGF and PDGF.

### Sequential Delivery Increases Ventricular Wall Thickness and Reduces Fibrosis in the Infarcted Myocardium

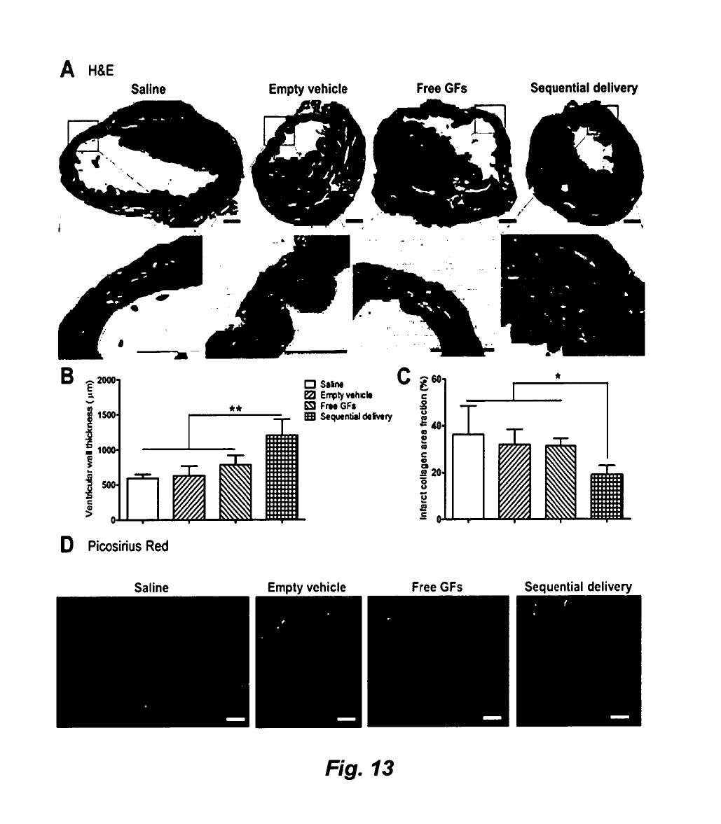

After evaluation of overall cardiac function, we performed investigations at the tissue level using histology and immunohistochemistry. At 4 weeks, H&E stained tissue showed increased granulated scar tissue areas with thinner LV walls in the infract region in saline (591.2±55.1 μm), empty vehicle (630±135.1 μm), and free GFs (797.9±144.3 μm) groups with no significant differences in wall thicknesses between them. In contrast, sequential delivery showed significantly increased LV wall thickness (1205.7±224.9 μm) compared to all groups with less scar tissue and granulation replacing normal cardiac muscle (FIG. 13(A,B)).

The extent of fibrosis was assessed using picosirius red staining. Collagen deposition was quantified and found to be significantly less in the sequential delivery group compared to all other groups which contained dense deposition of fibrillar collagen along the LV wall and extended to the infarct border zone (FIG. 13(C,D)). The area fractions of collagen deposition were 36.4±12.1% for saline, 32±6.6% for empty vehicle, 31.4±3.2% for free GFs, and 19±3.8% for sequential delivery (FIG. 13(C)). The reduced fibrosis and LV wall thinning due to sequential delivery of VEGF and PDGF is likely a contributing factor to the enhanced cardiac contractility since less fibrotic tissue reduces the stiffening of the ventricular walls and the extent of cardiac remodeling that occurs after MI (M. G. Sutton, et al., Left ventricular remodeling after myocardial infarction: pathophysiology and therapy, Circulation 101 (2000) 2981-2988).

### Sequential Delivery Provides Persistent Angiogenesis in the Infarcted Myocardium

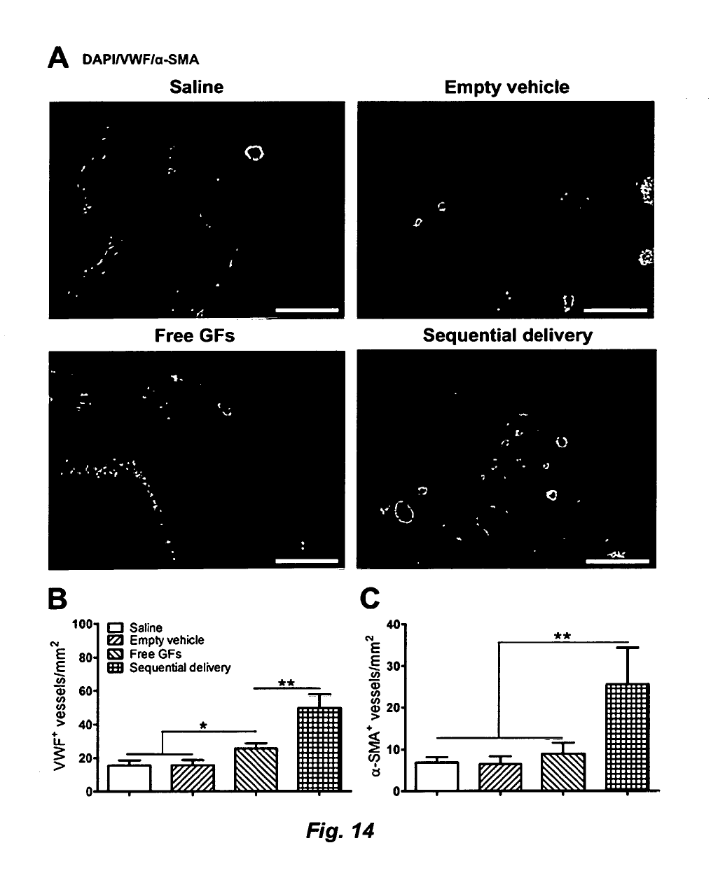

Restoring blood flow to the infarcted myocardium through robust angiogenesis is key to tissue regeneration and functional recovery. To investigate the development of mature and stable vasculature in the infarct region, we stained for the EC marker vWF and pericyte marker α-SMA (FIG. 14(A)). In addition to being an EC marker, vWF is a marker of cell homeostasis and can be used to evaluate the functionality of new blood vessels. After 4 weeks, free GFs group (25.6+3.2 per mm²) showed a significantly higher number of vWF-positive vessels in comparison to saline (15.5±3.1 per mm²) and empty vehicle (15.7±3 per mm²) groups which showed only few vessels in the infarct zone (FIG. 14(A,B)). In contrast, sequential delivery (49.6±8.1 per mm²) showed an increase in vWF-positive vessels that was significantly higher than all groups. This suggests that sequential release of VEGF and PDGF helped improve the formation of neovessels with increased functionality.

The stability and maturity of new vasculature and prevention of its regression is very important for successful ischemic tissue repair. The goal of therapeutic angiogenesis is therefore to produce neovasculature that is not transient but rather is long-term, stable, mature, and robust. To examine the maturity of neovessels, we stained for α-SMA to detect pericytes associated with newly formed vWF-positive vessels (FIG. 14(A)). Few α-SMA-positive vessels were found in saline (6.9±1.3 per mm), empty vehicle (6.5±1.9 per mm²), and free GFs (8.9±2.7 per mm²) groups with no statistical difference among them. On the other hand, sequential delivery showed many α-SMA-positive vessels (25.5±8.7 per mm²) likely due to the recruitment of pericytes by PDGF released in a sustained manner by the fibrin gel-coacervate delivery system (FIG. 14(A,C)). These results indicate the formation of stable and mature neovessels including capillaries and arterioles that are likely involved in tissue perfusion. This robust angiogenesis process is seemingly a key factor in the observed improvement of cardiac contractility at the functional level.

### Sequential Delivery Maintains Cardiac Viability in the Infarcted Myocardium

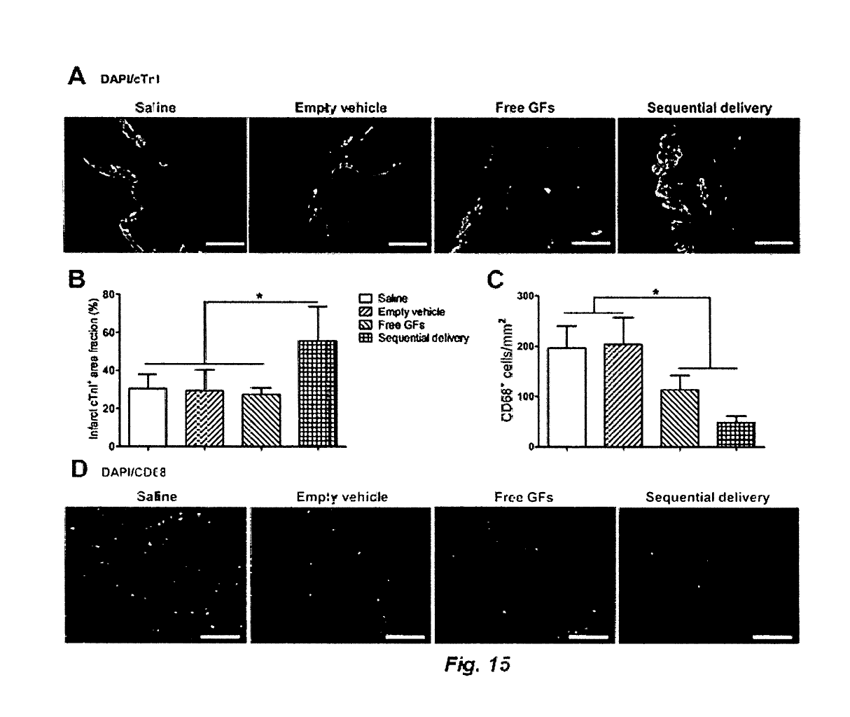

Cardiomyocyte survival is essential to maintain proper contractile function of the LV after MI. The viability of the cardiac muscle in the infarcted myocardium was examined by staining for cardiomyocyte marker cTnI (FIG. 15(A)). At 4 weeks, Saline, empty vehicle, and free GFs groups showed reduced cardiomyocyte survival in the infarct region with cTnI-positive area fractions of 30.5±7.4%, 29.4±11%, and 27.4±3.7%, respectively (FIG. 15(A, B)). There was no statistical differences noted among the three groups. In contrast, sequential delivery showed a significantly higher cTnI-positive area fraction (55.6±18.2%) than all groups suggesting better viability and preservation of the cardiac myofibers which help in the improvement of overall cardiac function (FIG. 15(A, B)).

### Sequential Delivery Reduces Inflammation in the Infarcted Myocardium

Reducing inflammation triggered by MI is an important goal towards recovery and repair of the myocardium (P. Krishnamurthy, et al., IL-10 inhibits inflammation and attenuates left ventricular remodeling after myocardial infarction via activation of STAT3 and suppression of HuR, Circ Res 104 (2009) e9-18). Local inflammation in the infarct zone was evaluated by staining for a pan-macrophage marker, CD68. At 4 weeks, we observed that both free GFs and sequential delivery groups greatly reduced the presence of macrophages (FIG. 15(C, D)). Sequential delivery group showed many less CD68-positive cells (48.9±12.4 per mm²) than free GFs group (113.6±28.2 per mm²), however no statistical difference was found between the two, though a trend is clearly observed. Saline (196.2±44.4 per mm²) and empty vehicle (204.2±52.7 per mm²) groups showed significantly higher numbers of CD68-positive cells (FIG. 15(C, D)). This result suggests an indirect role for VEGF and/or PDGF in reducing macrophage infiltration into the infarct zone after MI possibly due to reduced tissue damage or down-regulation of pro-inflammatory cytokines.

The delivery system described in this study is based on a combination of fibrin gel and a complex coacervate for sequential delivery of VEGF followed by PDGF. The coacervate contains heparin and a biocompatible polycation, PEAD, which closely and advantageously imitates the native signaling environment involving extracellular matrix proteoglycans, ligands, and cell receptors. This vehicle can protect the growth factors from rapid enzymatic degradation and potentiate their bioactivities.

In this study, we demonstrated that the fibrin gel-coacervate system achieved early release of VEGF to trigger EC proliferation and sprouting and delayed release of PDGF to recruit pericytes that stabilize the newly formed vessels. Even though PDGF is still present in the early stage, our delivery system largely limited its overlap with VEGF presence and thus limited the antagonism between the two factors. Our in vitro assays demonstrated that PDGF coacervate significantly improved SMC proliferation and migration compared to free PDGF. We also showed the importance of sequential delivery of VEGF followed by PDGF towards EC proliferation by limiting PDGF-mediated inhibition of VEGF angiogenic effects, in accordance with previous reports. The benefit of sequential release was further demonstrated by improved microvasculature sprouting from rat aortic rings.

In vivo, we demonstrated using a rat MI model that the fibrin gel-coacervate system led to a robust angiogenic response with extensive formation of mature and functional blood vessels in the infarct zone. We observed a significant increase in the number of vWF- and α-SMA positive vessels reflecting the formation of new stable and mature vasculature. Our results further demonstrate a reduction in myocardial fibrosis which mitigates the loss in contractile function seen in control groups (M. G. Sutton, et al., Circulation 101 (2000) 2981-2988 and P. Krishnamurthy, et al., Circ Res 104 (2009) e9-18). Moreover, cardiomyocyte survival, essential for preserving contractile function, was improved as a result of sequential delivery of VEGF and PDGF. Several variables not investigated in this study may have played a role in the improvement of cardiomyocyte survival and angiogenesis. For example, VEGF has been shown to elevate the levels of nitric oxide (L. Morbidelli, et al., Nitric oxide mediates mitogenic effect of VEGF on coronary venular endothelium, Am J Physiol 270 (1996) H411-415 and R. van der Zee, et al., Vascular endothelial growth factor/vascular permeability factor augments nitric oxide release from quiescent rabbit and human vascular endothelium, Circulation 95 (1997) 1030-1037), which is a potent vasodilator and an endothelial survival factor that prevents apoptosis and improves EC proliferation and migration (J. P. Cooke, et al., Nitric oxide and angiogenesis, Circulation 105 (2002) 2133-2135). Vasodilation soon after infarction may improve cardiomyocyte survival. VEGF also improves FGF-2-mediated angiogenesis (N. Maulik, et al. Growth factors and cell therapy in myocardial regeneration, J Mol Cell Cardiol 44 (2008) 219-227) and induces the release of SDF1-α which promotes cardiac stem cell and other progenitor cell mobilization to the infarct region (J. M. Tang, et al., VEGF/SDF-1 promotes cardiac stem cell mobilization and myocardial repair in the infarcted heart, Cardiovasc Res 91 (2011) 402-411). In addition to its role in stabilizing neovessels, PDGF can also activate cardioprotective signaling pathways in cardiomyocytes (P. C. Hsieh, et al., Controlled delivery of PDGF-BB for myocardial protection using injectable self-assembling peptide nanofibers, J Clin Invest 116 (2006) 237-248). Maintaining a viable cardiac muscle is essential to improving cardiac function after MI as demonstrated in studies attempting to stimulate proliferation of cardiomyocytes, prevent their apoptosis, and recruit cardiac progenitor cells to the heart. It is demonstrated herein that sequential delivery of VEGF and PDGF reduced the presence of macrophages in the infarct zone 4 weeks after MI. This reduction might be due to indirect VEGF and/or PDGF down-regulation of pro-inflammatory cytokines. It is also possible that the improved angiogenesis and better preservation of cardiac muscle observed in our study reduced tissue damage, which may have in turn reduced inflammation. The culmination of these many benefits was reflected on a functional level by improved cardiac contractility as early as 2 weeks after infarction with approximately 60% improvement over free GF delivery.

Many studies have investigated different types of delivery vehicles for spatiotemporal control over the release or expression of two or more growth factors; however very few have been tested in an animal model of MI. In the one study testing sequential delivery of VEGF and PDGF in the infarcted myocardium, an increased systolic velocity-time integral, a measure of displacement of the myocardium during contraction, was reported but surprisingly no significant improvement in ejection fraction or LV end-systolic dimension was observed compared to saline control or single GF delivery (X. Hao, et al. Angiogenic effects of sequential release of VEGF-A165 and PDGF-BB with alginate hydrogels after myocardial infarction, Cardiovasc Res 75 (2007) 178-185). Our study demonstrates significantly improved cardiac function through the measurement of LV contractility based on the FAC parameter, which is similar to ejection fraction but is a two-dimensional measurement. This functional improvement is corroborated by comprehensive histological and immunohistochemical analyses showing the beneficial effects of sequential delivery of VEGF and PDGF at the tissue level of the infarct region.

In conclusion, this Example demonstrates that sequential controlled release of VEGF₁₆₅ and PDGF-BB can trigger the formation and stabilization of neovasculature, and improve cardiac function after MI in a rat model. The improvement is observed at 2 weeks and maintained at a similar level at 4 weeks. Improvements at the tissue level include increased mature blood vessel formation, cardiomyocyte survival, and decreased collagen deposition and inflammation in the infarct zone. These results suggest that the fibrin gel-coacervate delivery system can induce robust angiogenesis, reduce scar burden, and potentially halt the pathological progression post MI. This controlled delivery approach warrants further investigation in a clinically-relevant large animal model.

### Example 4—Development of a Comprehensive Cardiac Repair Approach by Spatiotemporal Delivery of Complementary Proteins

After a heart attack, the infarcted myocardium is in dire need for repair and regeneration to reestablish functionality and prevent death. Protein signaling plays a pivotal role in the natural tissue regeneration and repair process. With multiple pathologies developing after myocardial infarction (MI), there is an urgent need for a comprehensive controlled release strategy that delivers therapeutic proteins to prevent or reverse these pathologies. Here, we studied the combination of four complementary factors: tissue inhibitor of metalloproteinases 3 (TIMP3) and interleukin-10 (IL-10) were embedded in a fibrin gel for early release, while basic fibroblast growth factor (FGF-2) and stromal cell-derived factor 1 alpha (SDF-1α) were embedded in heparin-based coacervates and distributed inside the same gel for a more sustained release. The efficacy of this approach was tested in a rat MI model and we report its significant ability to drive revascularization, cardiomyocyte survival, and stem cell homing, reduce remodeling, dilation, inflammation, fibrosis, myocardial strain, and extracellular matrix (ECM) degradation; and improve overall contractile function of the heart.

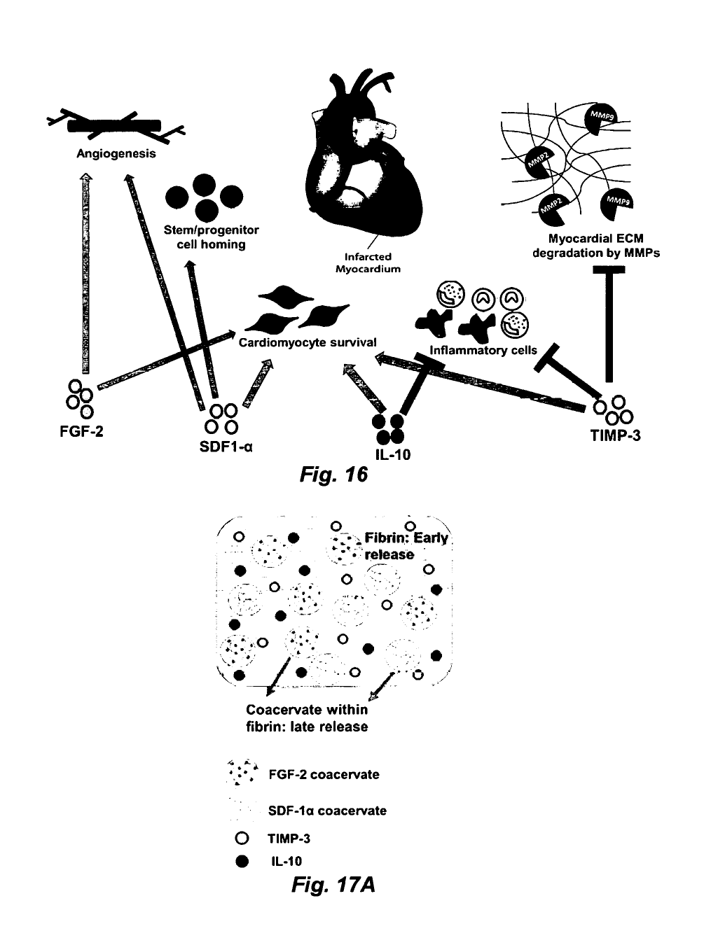

In this work, we explored the efficacy of the controlled and timed release of a combination of complementary proteins, which are relatively distinct in their cardiac functions. TIMP-3, IL-10, FGF-2, and SDF-1α are proteins with therapeutic potential in cardiac repair and regeneration (FIG. 16). TIMP-3 inhibits the activity of matrix metalloproteinases (MMPs) which cleave ECM proteins. Therefore, TIMP-3 might have an essential role in reducing ECM degradation early after infarction. IL-10 is an anti-inflammatory cytokine that has been shown to suppress infiltration of inflammatory cells into the myocardium and prevent cardiomyocyte apoptosis. FGF-2 plays a chief role in formation of neovasculature by inducing the proliferation, migration, and differentiation of vascular cells and enhancing the signaling of other angiogenic factors. SDF-1α has been shown to trigger cardiomyocyte survival and recruit stem cells to the infarct region. TIMP-3 and IL-10 were intended to modulate, but not eliminate, inflammation and ECM degradation soon after MI. FGF-2 and SDF-1α were intended to promote angiogenesis and recruit progenitor cells into the infarct at the later stage of the repair process. A composite hydrogel was designed comprised of fibrin gel and heparin-based coacervate to achieve the sequential release of TIMP-3 and IL-10 followed by FGF-2 and SDF-1α. To achieve this controlled release, TIMP-3 and IL-10 were encapsulated in fibrin gel to offer early release, while FGF-2 and SDF-1α were encapsulated in heparin-based coacervates and distributed in the same fibrin gel to offer a sustained release.

The endogenous biological system and tissue repair process are intrinsically very complex with many proteins involved and possible interactions between them. Considering the four proteins of interest and the control combinations that can result from them, it is time-, money-, resource-, and labor-consuming to test all possible protein combinations and dosages. To address this challenge, the design of experiment (DOE) tool, known as fractional factorial design, can be a powerful statistical method to reduce study groups in biomedical research. Fractional factorial designs are common in scientific studies and industrial applications, and have been used effectively. However, they have not been taken advantage of as commonly in biomedical research. These designs have been utilized previously to study drug combinations for treating Herpes simplex virus type 1 (HSV-1) and as a method to investigate the effects of different processing parameters for a tissue-engineered scaffold. Fractional factorial designs allow us to build statistical models using a small number of runs. Such models can help us identify important proteins, protein interactions, protein dosages, and optimal protein combinations.

Using fractional factorial design, this initial combination of four proteins was optimized based on the contribution and dosage of each protein to improve cardiac function after MI. The factorial design results showed significant contributions of TIMP-3, FGF-2, and SDF-1α in improving cardiac function 4 weeks after MI. The optimized protein combination was then tested in an expanded study for efficacy in cardiac repair and regeneration post infarction. It was demonstrated that the controlled and timed release of TIMP-3, FGF-2, and SDF-1α at optimized dosages can significantly improve cardiac function and repair. Functional and histological evaluations were performed at 2 and/or 8 weeks after MI in a rat model. Improvements at the tissue level are reported in increased angiogenesis, cardiomyocyte survival, and stem cell homing, and reduced myocardial strain levels, ventricular dilation, ECM degradation, inflammation, fibrosis, MMP activity, and cell apoptosis. We demonstrate, for the first time, that a more comprehensive therapy of controlled delivery of complementary proteins can mitigate the MI injury and set into motion a robust cardiac tissue repair and regeneration process, giving hope of driving the functional and structural recovery of the infarcted heart to a new level.

### Materials and Methods

### Release Assay of Complementary Proteins

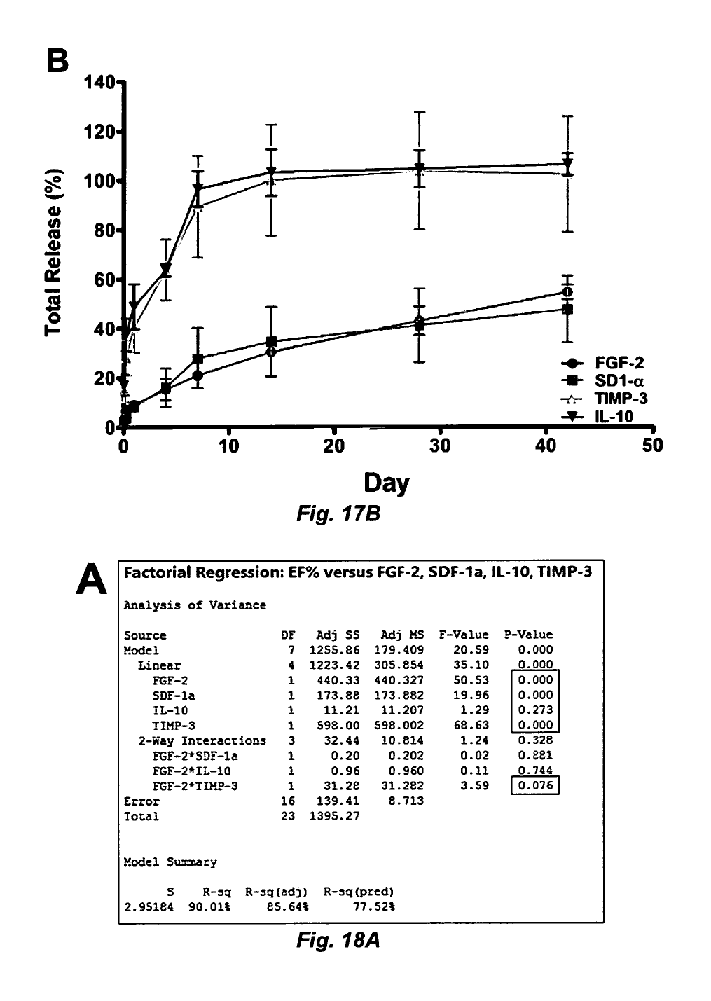

Poly(ethylene argininylaspartate diglyceride) (PEAD) was synthesized as previously described (Chu H, et al. Design, synthesis, and biocompatibility of an arginine-based polyester. Biotechnol Prog 2012; 28:257-64). The release assay was performed using 100 ng of each of TIMP-3 (R&D Systems, Minneapolis, Minn.), IL-10, FGF-2, and SDF-1α (PeproTech, Rocky Hill, N.J.). All solutions were prepared in 0.9% saline. FGF-2 and SDF-1α coacervates were made by mixing 1 μl of 100 ng/μl for each of FGF-2 and SDF-1α with 2 μl of 5 mg/ml heparin first (Scientific Protein Labs, Waunakee, Wis.), then with 2 μl of 25 mg/ml of the polycation, poly(ethylene arginyl aspartate diglyceride) (PEAD), at PEAD:heparin:protein mass ratio of 250:50:1. This created 6 μl of FGF-2/SDF-1α coacervates. Fibrin gel-coacervate vehicle was made by mixing 80 μl of 20 mg/ml fibrinogen (Sigma-Aldrich, St. Louis, Mo.), 2 μl of 5 mg/ml heparin, 1 μl of 100 ng/μl for each of TIMP-3 and IL-10; then the 6 μl FGF-2/SDF-1α coacervates were added, followed by 5 μl of 1 mg/ml aprotonin (Sigma-Aldrich, St. Louis, Mo.). Lastly, 5 μl of 1 mg/ml thrombin (Sigma-Aldrich, St. Louis, Mo.) was added to induce gelation, resulting in a 100 μl fibrin gel-coacervate vehicle (FIG. 17A). A 100 μl of 0.9% saline was deposited on top of fibrin gel to be collected at 1 h, 16 h, 1, 4, 7, 14, 28, and 42 days. The samples (n=3) were incubated at 37° C. After centrifugation at 12,100 g for 10 min, supernatant was collected and stored at −80° C. to detect amount of released GFs by sandwich enzyme-linked immunosorbent assay (ELISA) kits (PeproTech, Rocky Hill, N.J.) (R&D Systems, Minneapolis, Minn.). The absorbance at 450/540 nm was measured by a SynergyMX plate reader (Biotek, Winooski, Vt.). Normalizing standards (n=3) were prepared using the same amounts of free proteins in 100 μl of 0.9% saline.

### Two-Level Half Fractional Factorial Design

We formulated a two-level half fractional factorial design to select the combination of proteins and their dosages that are the most effective at recovering cardiac function post MI. The two levels refer to upper and lower doses for each protein and were chosen based on previous experiments and literature review. The half fractional factorial design means half of the total runs were performed. For this design, we can estimate all main effects and some 2-factor interactions, which is quite reasonable in practice to evaluate the significance of each protein in the combination and find the corresponding optimal dose to use in the expanded study (Wu C-F, Hamada M. Experiments: planning, analysis, and parameter design optimization. New York: Wiley; 2000).

Using the design formula 2^{(k-p)} with k=4 factors, and p=1 (for half fractional factorial), we got 2³=8 dosage groups. From our previous studies, preliminary results, and literature, we selected the most commonly used dose for each protein as the high doses and one-fifth of that as the low doses. The high doses for FGF-2, SDF-1α, IL-10, and TIMP-3 were 3, 3, 2, and 4 μg respectively; while the lower doses are 0.6, 0.6, 0.4, and 0.8 μg respectively (Table 2). TIMP-3 and IL-10 were encapsulated in fibrin gel, while FGF-2 and SDF-1α were encapsulated in heparin-based coacervates and distributed in the same fibrin gel (FIG. 17A). With n=3 per dosage group and a sham group, we utilized 27 rats for this initial-stage study. Using a statistical software (Minitab, State College, Pa.), this initial-stage design provided a table showing eight groups of varying protein doses that need to be tested (Table 1). Each group with varying protein doses was tested in a rat acute MI model. The key outcome measurement of cardiac function was ejection fraction (EF %) computed using MRI at 4 weeks post-MI. Once the ejection fractions were measured and input in Minitab, we performed detailed statistical analysis that provided us the necessary information about the relative importance of each protein and its optimal dose in the context of improving cardiac function after MI. The results and analysis of this experiment allowed us to proceed to the expanded study with a refined combination and doses of the proteins.

Rat Acute Myocardial Infarction Model MI and injections were performed as previously described (Dobner S, et al. A synthetic non-degradable polyethylene glycol hydrogel retards adverse post-infarct left ventricular remodeling. J Card Fail 2009; 15:629-36). Briefly, 6-7 week old (175-225 g) male Sprague-Dawley rats (Charles River Labs, Wilmington, Mass.) were anesthetized first then maintained with 2% isoflurane at 0.3 L/min (Butler Schein, Dublin, Ohio), intubated, and connected to a mechanical ventilator to support breathing during surgery. The body temperature was maintained at 37° C. by a hot pad The ventral side was shaved and a small incision was made through the skin. Forceps, scissors, and cotton swabs were used to dissect through the skin, muscles, and ribs. Once heart was visible, the pericardium was torn. MI was induced by permanent ligation of the left anterior descending (LAD) coronary artery using a 6-0 polypropylene suture (Ethicon, Bridgewater, N.J.). Infarct was confirmed by macroscopic observation of a change in color from bright red to light pink in the area below the ligation suture. Five minutes after the induction of MI, different treatment and control solutions were injected intramyocardially at 3 equidistant points around the infarct zone using a 31 G needle (BD, Franklin Lakes, N.J.).

For the fractional factorial design optimization study, 100 μl of fibrin gel-coacervate vehicle was prepared briefly as follows: 18 μl coacervate solution (PEAD:heparin:protein mass ratio at 50:10:1) containing respective dosages of FGF-2 and SDF-1α as outlined in Table 1, 65 μl of 20 mg/ml fibrinogen, 10 μl of solution containing heparin and respective dosages of TIMP-3 and IL-10 as outlined in Table 1, 5 μl of 1 mg/ml aprotonin (Sigma-Aldrich, St. Louis, Mo.). Lastly, 2 μl of 1.5 mg/ml thrombin (Sigma-Aldrich, St. Louis, Mo.) was added and the total solution was injected shortly before gelation occurred, approximately 50 seconds after mixing (FIG. 17A). The chest was closed and the rat was allowed to recover. After 4 weeks, all animals (n=27) were imaged using cardiac MRI and sacrificed.

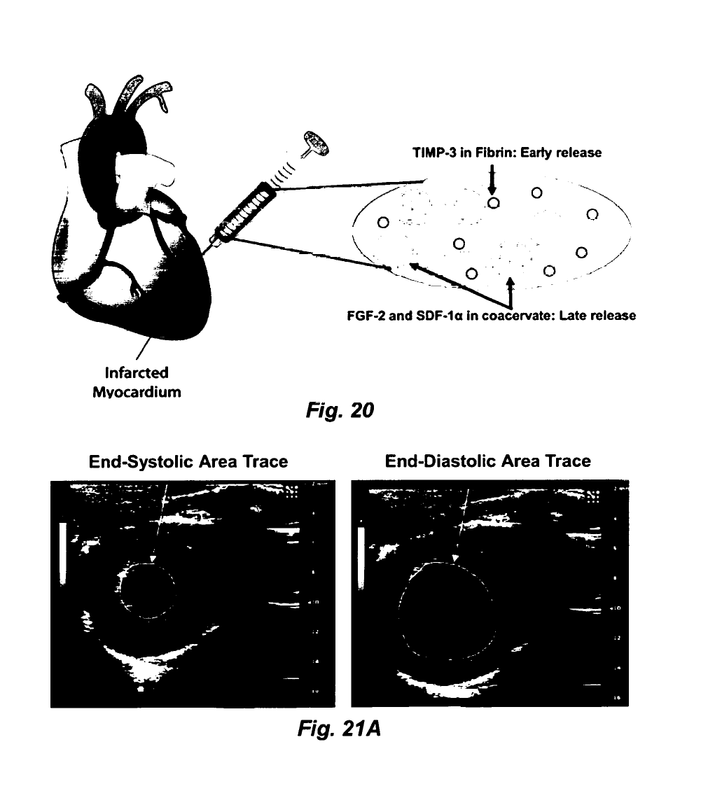

For the expanded study with refined protein combination and dosages, four groups (n=56 rats) were studied: sham, saline, free proteins, and delivered proteins. Empty vehicle (empty fibrin gel-coacervate composite) was not tested as a control in this study as it has shown no difference to saline in our previous work. The sham group (n=13) underwent the surgery in which the heart was exposed and pericardium was torn, then chest was closed and rat recovered. The saline group (n=14) underwent the surgery in which MI was induced and 100 μl of 0.9% sterile saline was injected around the infarct region. The free proteins group (n=14) underwent the surgery in which MI was induced and 100 μl of 0.9% sterile saline containing 3 μg each of free TIMP-3, FGF-2, and SDF-1α was injected around the infarct region. The delivered proteins group (n=15) underwent the surgery in which MI was induced and 100 μl of fibrin gel-coacervate vehicle was injected around the infarct region. The fibrin gel-coacervate composite was prepared briefly as follows: 18 μl coacervate solution containing 3 μg each of FGF-2 and SDF-1α, 67 μl of 20 mg/ml fibrinogen, 6 μl of solution containing heparin and 3 μg of TIMP-3, 5 μl of 1 mg/ml aprotonin (Sigma-Aldrich, St. Louis, Mo.). Lastly, 4 μl of 1.5 mg/ml thrombin (Sigma-Aldrich, St. Louis, Mo.) was added and the total solution was injected shortly before gelation occurred, approximately 40 seconds after mixing (FIG. 20B). All solutions were prepared in 0.9% sterile saline. The chest was closed and the rat was allowed to recover. At multiple time points, rats were imaged using echocardiography. At 8 weeks, a subset was imaged using cardiac MRI. After 2 (n=17) or 8 weeks (n=39), animals were sacrificed and hearts were harvested for histological, immunohistochemical, and western blot evaluations.

### Echocardiography

At pre-MI, 1, 2, 5, and 8 weeks post-MI, rats (n=9-10 per group) were anesthetized then maintained with 1-1.5% isoflurane gas throughout the echocardiographic study. Rats were placed in the supine position, immobilized on a heated stage equipped with electrocardiography, and the hair in the abdomen was removed. The body temperature was maintained at 37° C. Short-axis videos of the left ventricle (LV) by B-mode were obtained using a high-resolution in vivo micro-Imaging system (Vevo 2100, Visual Sonics, Ontario, Canada) equipped with a high-frequency linear probe (MS400, 30 MHz) (FUJIFILM VisualSonics, Canada). End-systolic (ESA) and end-diastolic (EDA) areas were measured using NIH ImageJ 1.46r and fractional area change (FAC) was calculated as: [(EDA−ESA)/EDA]×100%. Percent improvements of one group over another were calculated as the difference between the % drops in FAC values of the first and second groups divided by the higher % drop of the two groups.

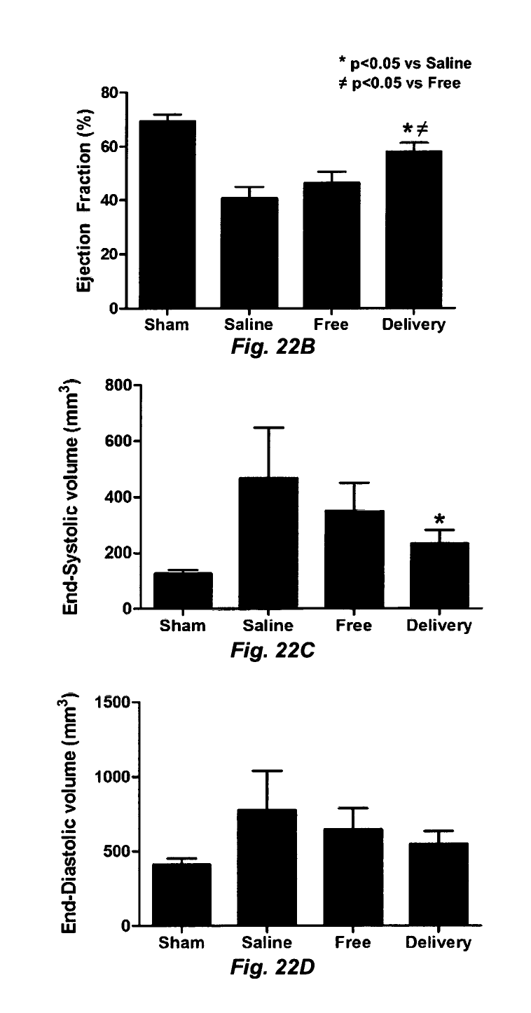

Myocardial strain level measurements: The ultrasound B-mode frames of LV short-axis view acquired at 8 weeks post-MI were analyzed (n=5 rats per group) using a strain analysis algorithm (VevoStrain™, Vevo2100). Five regions of interest (ROI) were selected along the LV mid-wall including one ROI in the anterior lateral (infarcted area) and four ROIs in the anterior medial, septal, posterior, posterior lateral (unaffected areas) walls of the LV (FIG. 22A). The peak strain in the infarcted area was normalized to the average peak strains of the four ROIs in unaffected LV walls during a segment of full cardiac cycles. Both radial and circumferential strains were computed. The radial strain is defined as the percent change in myocardial wall thickness, and the circumferential strain is defined as the percent change in myocardial circumference.

### Cardiac Magnetic Resonance Imaging

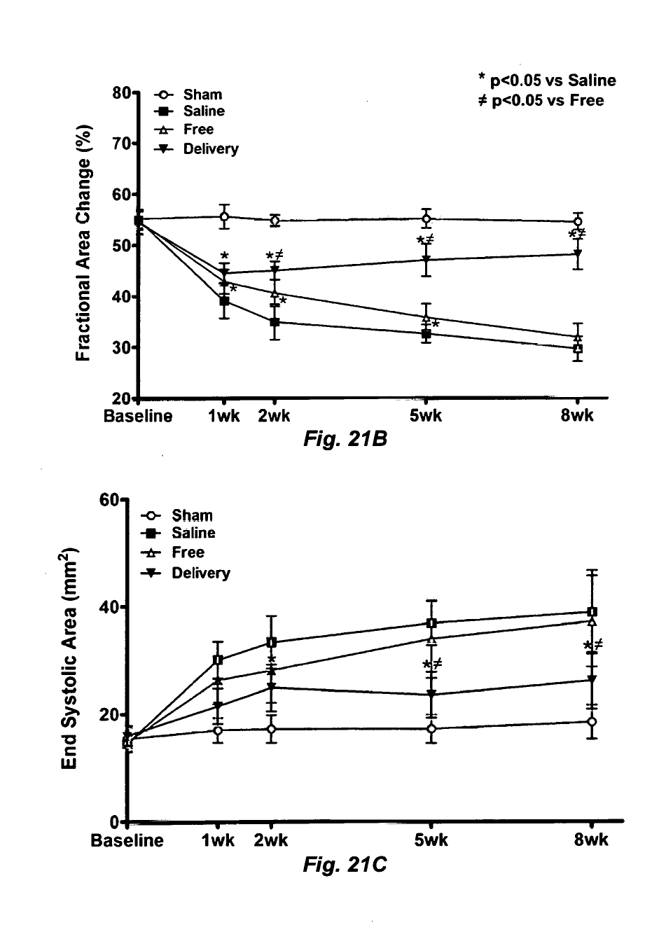

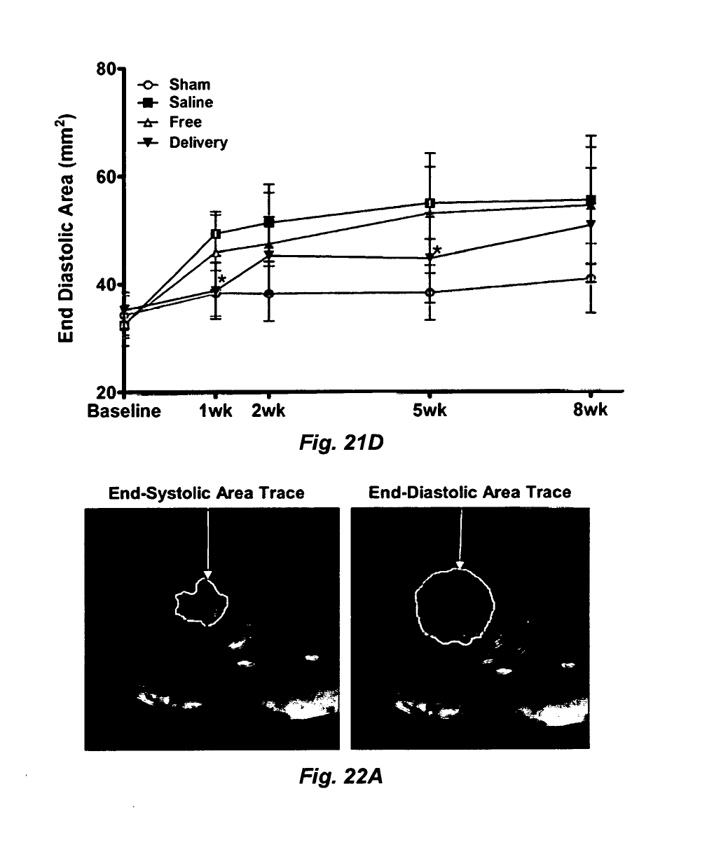

Cine cardiac magnetic resonance imaging (MRI) imaging was used as previously indicated (Slawson S E, et al. Cardiac MRI of the normal and hypertrophied mouse heart. Magn Reson Med 1998; 39:980-7 and van Rugge F P, et al. Magnetic resonance imaging during dobutamine stress for detection and localization of coronary artery disease. Quantitative wall motion analysis using a modification of the centerline method. Circulation 1994; 90:127-38). Here we implemented cine imaging to measure LV volumes and ejection fraction from infarcted rat hearts at 8 weeks (n=5-8 per group). The rats were induced with isoflurane gas, intubated and ventilated at 1 mL/100 g of body weight with 2% isoflurane in 2:1 O₂:N₂O gas mixture and 60 BPM. Animals were placed on an MRI compatible bed and positioned in the MRI magnet. While in the MRI magnet, rectal temperature was monitored and maintained at 37° C., and ECG and respiration were also monitored. Following pilot scans, cine imaging was used to obtain short-axis images covering the entire heart volume and the whole cardiac cycle. The field of view was approximately 4 cm×4 cm with a 256×256 matrix, 156 μm plane resolution. Approximately 10 slices were collected to cover the area between the heart apex to the mitral valves with 1.5 mm slice thickness. ESA and EDA were measured from every slice using NIH ImageJ 1.46r and multiplied by slice thickness to measure the respective volumes (FIG. 21A). Individual volumes were added cumulatively to measure the overall end-systolic (ESV) and end-diastolic (EDV) volumes. These volumes were used to compute ejection fraction as EF %=[(EDV−ESV)/EDV]×100%. Percent improvements of one group over another were calculated as the difference between the % drops in EF values of the first and second groups divided by the higher % drop of the two groups.

### Histological Analysis

At 2 weeks (n=4-5 per group) and 8 weeks (n=5-7 per group) post-infarction, rats were sacrificed by injecting 2 ml of saturated potassium chloride (KCl) solution (Sigma Aldrich, St. Louis, Mo.) in the LV to arrest the heart in diastole. Hearts were harvested, fixed in 2% paraformaldehyde (fisher Scientific, Fair Lawn, N.J.) for 1-2 hours, deposited in 30% sucrose solution (w/v) overnight, frozen in O.C.T compound (Fisher Healthcare, Houston, Tex.), and stored at −20° C. until cryosectioning. Specimens were cryosectioned at 6 μm thickness from apex to the ligation level with 500 μm intervals. Hematoxylin and eosin (H&E) staining was performed for general evaluation. H&E stained slides were selected and the ventricular wall thickness in the infarct zone (n=3-4 per group at 2 wks, n=4-6 at 8 wks) was measured near the mid-section level of the infarct tissue using NIS Elements AR imaging software (Nikon Instruments, Melville, N.Y.).

For assessment of interstitial fibrosis, Picrosirius red staining was used to stain collagen fibers and image under polarized light showing up as metallic red color. The fraction area of collagen deposition in the cross-sectional area of the whole heart was measured by NIS Elements AR software near the mid-section level of the infarct tissue (n=3-5 per group at 2 wks, n=4-7 at 8 wks). An object count tool was used to include RGB pixels specific to the stained collagen fibers in the fraction area by defining a proper threshold value.

### Immunohistochemical Analysis

For evaluation of inflammation, a rabbit polyclonal antibody F4/80 (1:100, Santa Cruz Biotechnology, Dallas, Tex.), a pan-macrophage surface marker, was used followed by an Alexa fluor 594 goat anti-rabbit antibody (1:200, Invitrogen, Carlsbad, Calif.). Slides were also co-stained by a mouse anti-rat CD163 (1:150, Bio-Rad Laboratories, Hercules, Calif.), an M2 macrophage phenotype marker, followed by an Alexa fluor 488 goat anti-mouse antibody (1:200, Invitrogen, Carlsbad, Calif.). Slides were last counterstained with 4′,6-diamidino-2-phenylindole (DAPI) (Invitrogen, Carlsbad, Calif.). For quantification near the mid-section level of the infarct tissue, F4/80-positive cells and CD163-positive cells were counted in two opposite regions of the infarct border zone, averaged, and reported per mm² areas (n=3-4 rats per group at 2 wks).

For evaluation of cardiac muscle viability, a rabbit polyclonal cardiac troponin I (cTnI) antibody (1:200, US Abcam, Cambridge, Mass.) was used followed by an Alexa fluor 488 goat anti-rabbit antibody (1:200, Invitrogen, Carlsbad, Calif.). Slides were last counterstained with DAPI. The fraction area of viable cardiac muscle in the cross-sectional area of the whole heart was measured by NIS Elements AR software near the mid-section level of the infarct tissue (n=3-5 per group at 2 wks, n=5-6 at 8 wks). An object count tool was used to include RGB pixels specific to the stained viable cardiac muscle in the fraction area by defining a proper threshold value.

For evaluation of angiogenesis, endothelial cells (ECs) were detected by a rabbit polyclonal Von Willebrand factor (vWF) antibody (1:200, US Abcam, Cambridge, Mass.) followed by an Alexa fluor 594 goat anti-rabbit antibody (1:200). Mural cells were detected by a FITC-conjugated anti-α-smooth muscle actin (α-SMA) monoclonal antibody (1:500, Sigma Aldrich, St. Louis, Mo.). Slides were last counterstained with DAPI. For quantification near the mid-section level of the infarct tissue, vWF-positive vessels (defined as those with lumen) and α-SMA-positive vessels were counted in two opposite regions of the infarct border zone, averaged, and reported per mm² areas (n=3-4 rats per group at 2 wks, n=5-6 per group at 8 wks).

For evaluation of stem cell homing, stem/progenitor cells were detected by a rabbit polyclonal c-Kit antibody (1:100, Santa Cruz Biotechnology, Dallas, Tex.) followed by an Alexa fluor 488 goat anti-rabbit antibody (1:200). Slides were last counterstained with DAPI. For quantification near the mid-section level of the infarct tissue, c-Kit-positive cells were counted in two opposite regions of the infarct border zone, averaged, and reported per mm² areas (n=5 rats per group at 8 wks).

### Molecular Pathways Analysis by Western Blot

Rat hearts (n=15) were harvested and rapidly stored at −80° C. for western blotting. For protein extraction, myocardial specimens weighing approximately 100 mg were excised from the LV generating a composite material comprising a spectrum between normal, infarct, and borderzone tissue. The tissues were then homogenized at 0.2 μg/ml in a modified lysis RIPA buffer (50 mM Tris-HCl, 1% NP-40, 20 mM DTT, 150 mM NaCl, pH=7.4) with protease and phosphatase inhibitors. The complex was then centrifuged at 12,100 g for 10 min, and the supernatant was collected and stored at −80° C. until use.

For total protein content, the extracts above were quantified with Pierce 660 nm Protein Assay (Thermo Fisher Scientific, Waltham, Mass.). The equivalent of 100 μg protein was separated using 11.5% gel and then transferred onto a PVDF membrane (Bio-Rad Laboratories, Hercules, Calif.). The membrane was blocked with 5% BSA in TBS with 0.05% Tween 20 for 1 h, then incubated with following antibody solutions: AKT, p-AKT, ERK1/2, p-ERK1/2 (all at 1:300, Santa Cruz Biotechnology, Dallas, Tex.), cleaved caspase-3 (1:1,000, Cell Signaling Technology, Boston, Mass.), and GAPDH (1:5000, US Abcam, Cambridge, Mass.). The membranes were washed with TBS three times and incubated with secondary antibodies for 2 h at room temperature. Signals were visualized using the ChemiDic™ XRS+Imaging System (Bio-Rad Laboratories, Hercules, Calif.), and band densities were quantified with NIH ImageJ software (n=3 per group).

### Myocardial Protein Signaling Analysis by ELISA

The tissue lysates acquired in the western blot section (n=3-4 rats per group) were used for detecting the levels of IGF-1, VEGF, Shh, and TGF-β1 in the LV myocardium. Sandwich ELISA kits for VEGF and IGF-1 (PeproTech, Rocky Hill, N.J.) were used per the manufacturer's instructions. Lysates were diluted 1:20 for VEGF and 1:50 for IGF-I. For Shh and TGF-β1, indirect ELISA was run using rabbit polyclonal antibodies against Shh and TGF-β1 (both at 1:30, Santa Cruz Biotechnology, Dallas, Tex.) followed by a secondary biotinylated goat anti-rabbit IgG (1:100, Santa Cruz Biotechnology, Dallas, Tex.). Lysates were diluted 1:15 for Shh and 1:25 for TGF-β1. The absorbance at 450/540 nm was measured by a SynergyMX plate reader. Results were corrected to account for differences in total protein content of samples.

### MMP-2/9 Activity Assay

The tissue lysates acquired in the western blot section (n=3-4 rats per group) were used for detecting the activity of MMP-2/9 in the LV myocardium. The Calbiochem InnoZyme™ Gelatinase activity assay fluorogenic kit (EMD Millipore, Billerica, Mass.) was followed per the manufacturer's instructions. Briefly, lysate samples (diluted 1:2 in activation buffer) were incubated with a fluorogenic substrate solution that is highly selective for MMP-2 and MMP-9. Gelatinases in the sample lysates of the myocardium cleave the substrate, resulting in an increase in fluorescent signal measured at an excitation wavelength of 320 nm and an emission wavelength of 405 nm by a SynergyMX plate reader. The gelatinase control, activated similarly, was used at serial dilutions to create a standard curve for converting the fluorescence values of MMP activity to concentrations (ng/ml).

### Statistical Analysis

Results are presented as means±standard deviations (SD). GraphPad Prism 5.0 software (La Jolla, Calif.) and Minitab software (State College, Pa.) were used for statistical analysis. Statistical differences between groups were analyzed by one-way ANOVA (multiple groups) or two-way repeated ANOVA (repeated echocardiographic measurements) with 95% confidence interval. Bonferroni multiple comparison test was performed for ANOVA post-hoc analysis. Statistical significance was set at p<0.05.

### Results

### Fibrin Gel-Coacervate Composite Achieves Sequential Protein Release

We have reported the use of the fibrin gel-coacervate composite previously for the early release of VEGF followed by the late release of PDGF to induce therapeutic angiogenesis in the infarcted heart. In this study, we implement a more comprehensive strategy to prevent or reverse multiple pathologies developed after MI. We tested the fibrin gel-coacervate composite's ability for early release of TIMP-3 and IL-10 followed by late release of FGF-2 and SDF-1α. In order to obtain faster TIMP-3/IL-10 release, we embedded these two proteins in a fibrin gel directly, before gelation. We then embedded FGF-2 and SDF-1α within heparin-based coacervates distributed in the same fibrin gel to provide sustained release (FIG. 17A).

The loading efficiencies were 85% for TIMP-3, 83% for IL-10, 97% for FGF-2, and 98% for SDF-1α, as quantified 1 h after loading them into the composite. By day 1, approximately 40% of TIMP-3 and 50% of IL-10 have been released, reaching 90% and 97% total release respectively by one week (FIG. 17B). As for FGF-2 and SDF-1α, we observed a longer sustained release that lasted for many weeks due to their encapsulation within the coacervates inside the gel. By one week, only 21% of FGF-2 and 28% of SDF-1α were released, reaching 55% and 48% total release respectively by 6 weeks (FIG. 17B). This controlled release system achieved sequential release kinetics, where most of TIMP-3 and IL-10 amounts were released by 1 week compared to a sustained release of FGF-2 and SDF-1α that lasted at least 6 weeks. The in vivo release rates can be further influenced by one or more factors including fibrinolysis, enzymatic degradation by esterases and heparinases, hydrolytic degradation of the PEAD polycation, and dissociation of the coacervate in an ionic environment. Thus, in vivo release is expected to be faster. Overall, the release kinetics attained with the fibrin gel-coacervate composite reflect the desired goal of providing TIMP-3 and IL-10 early after MI to reduce ECM degradation and inflammation, while providing FGF-2 and SDF-1α in a more sustained fashion for triggering a robust neovasculature formation process and stem cell recruitment.

### Optimization of the Protein Combination and Doses

The efficacy of combination of TIMP-3, IL-10, FGF-2, or SDF-1α to promote cardiac repair post-MI is unknown. The significance of each protein in combination within the context of improving cardiac function and repair after MI also was unknown, as was their efficacy in controlled delivery systems, or the optimal dosage for each protein in free or controlled release form. Therefore, a two-level half fractional factorial design was used to build a statistical model with a small number of runs—saving time, resources, and money. This model provided valuable insight about the relative significance of each protein of the combination on improving cardiac function, and the optimal dose needed to impart potential benefit for cardiac repair and regeneration.

TABLE 4
Treatment groups according to two-level half fractional factorial
design and the corresponding ejection fraction obtained by MRI

| Protein | FGF-2 | SDF-1α | IL-10 | TIMP-3 | EF % | SD |
|---|---|---|---|---|---|---|
| Dose group 1 | 3 μg | 3 μg | 2 μg | 4 μg | 62.3 | 1.3 |
| Dose group 2 | 3 μg | 3 μg | 0.4 μg | 0.8 μg | 56.8 | 1 |
| Dose group 3 | 3 μg | 0.6 μg | 2 μg | 0.8 μg | 50.7 | 4.3 |
| Dose group 4 | 3 μg | 0.6 μg | 0.4 μg | 4 μg | 59.4 | 3.6 |
| Dose group 5 | 0.6 μg | 3 μg | 2 μg | 0.8 μg | 44.9 | 3.6 |
| Dose group 6 | 0.6 μg | 3 μg | 0.4 μg | 4 μg | 58.9 | 3.4 |
| Dose group 7 | 0.6 μg | 0.6 μg | 2 μg | 4 μg | 51.6 | 1.9 |
| Dose group 8 | 0.6 μg | 0.6 μg | 0.4 μg | 0.8 μg | 41.1 | 2.9 |
| Sham | | | | | 70.2 | 2.1 |

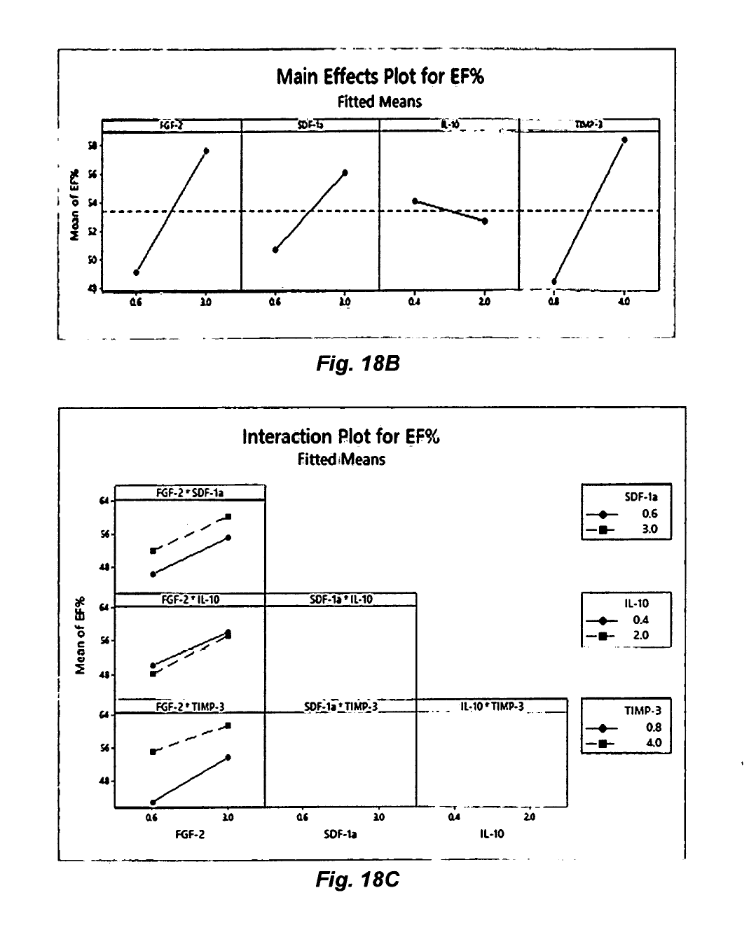

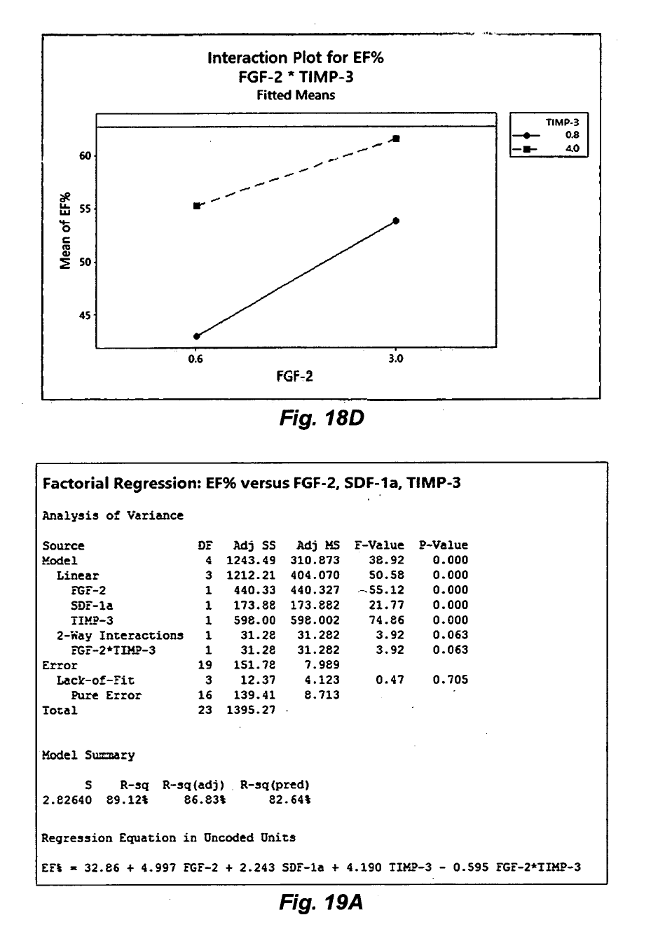

In this optimization study, we generated, using statistical software, a table organizing 8 groups with different upper and lower doses for each protein, to be tested for efficacy on ejection fraction (EF %) in a rat MI model (Table 4). Results demonstrated the significant main effects of TIMP-3, FGF-2, and SDF-1α (p<0.001), while suggesting little effect of IL-10 (p=0.273) on improvement of cardiac function (FIG. 18A). This means that, within the context of improving EF % using controlled delivery of this combination, TIMP-3, FGF-2, and SDF-1α are beneficial for improving cardiac function while IL-10's effect is minimal, and thus can be removed for the expanded study with refined combination. Although IL-10 is a strong anti-inflammatory cytokine, its minimal effect on improving cardiac function within the context of this protein combination might be due to the more important presence of TIMP-3, which has been shown to have some anti-inflammation effects.

As for the optimal protein doses, the most effective group (group 1) restored EF to 62%, which is closer to the average of 3 sham controls at 70% than the remaining 7 groups. We observed, from the main effects plot, that all of the estimates for FGF-2, SDF-1α, and TIMP-3 have positive coefficients while IL-10 has a negative coefficient, implying that larger cardiac function improvement can be achieved by using the higher dosages of FGF-2, SDF-1α, and TIMP-3 (FIG. 18B). Since IL-10's effect is not significant, we concluded that removing it from the combination is the better decision than using it at a low dosage (FIGS. 18A and 18B).

TIMP-3 had the greatest main effect on improvement of EF % accounting for 43% of the total sum of squares (598/1395), followed by FGF-2 accounting for 32% (440/1395) of the total sum of squares, then SDF-1α accounting for 12.5% (174/1395) of the total sum of squares. Together, the main effects of these proteins dominate the system and account for 87.5% of the total sum of squares, suggesting that the individual effects of these 3 proteins are responsible for 87.5% of the variation in EF %, while higher-order protein interactions and error account for 12.5% of that variation (FIG. 18A). The effects of the 2-way protein interactions, that this factorial design was able to estimate, are all insignificant (FIG. 18A,C). However, the interaction between FGF-2 and TIMP-3 was worth paying attention to since it approached significance value (p=0.076). This interaction suggests slight antagonism between the 2 proteins (FIG. 18D). When TIMP-3 is more active, that is present at a higher dose, the effect of FGF-2 on EF % is less than when TIMP-3 is less active, present at a lower dose. This can be observed by the lower positive slope of the interaction at a high dose of TIMP-3 compared to the lower dose, although the EF % mean is remarkably greater at a high dose of TIMP-3 than a low dose.

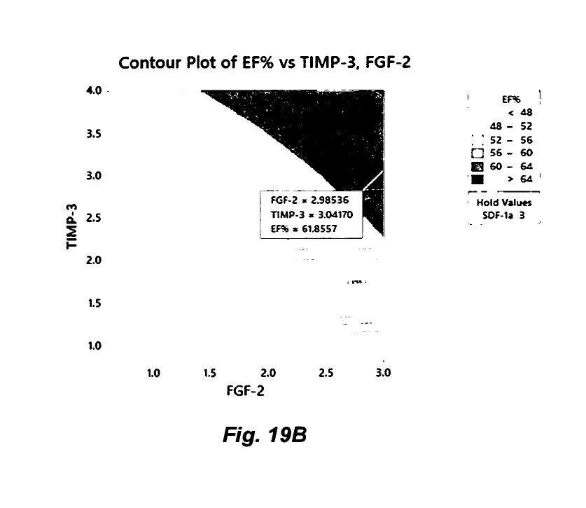

Taking these statistical results and the commercial cost of each protein into consideration, we decided to move forward with FGF-2, SDF-1α, and TIMP-3 in the protein combination with a dosage of 3 μg for each, thereby keeping the upper doses of FGF-2 and SDF-1α, while reducing TIMP-3 upper dose slightly from 4 μg to 3 g (FIG. 20). Modifying the statistical model to accommodate these changes gave us a new regression equation that we used to predict an EF % of approximately 62% if we utilize FGF-2, SDF-1α, and TIMP-3 at 3 μg each in the designed scheme within the fibrin gel-coacervate composite laid out previously (FIGS. 19A and 19B).

### Spatiotemporal Protein Delivery Improves Cardiac Function and Reduces Dilation

After refining the protein combination and dosages, we evaluated the in vivo effect of spatiotemporal delivery of TIMP-3, FGF-2, and SDF-1α (3 μg each) using the fibrin gel-coacervate composite in a rat MI model comparing sham, saline, free proteins, and delivered proteins (FIG. 20). Empty vehicle (empty fibrin gel-coacervate composite) was not tested as a control in this study as it has shown no difference to saline. We evaluated changes in LV contractility as a measure of heart function. Fractional area change (FAC) was computed from measured end-systolic (ESA) and end-diastolic (EDA) areas of 2-D echocardiography videos (FIG. 21A). Sham group maintained an FAC value of approximately 55% at all time points (FIG. 21B). At 1 week post-infarction, FAC values of saline, free, and delivery groups dropped significantly, however, both delivery and free values were significantly higher than saline (p<0.01). This suggests that the 3-protein therapy, whether free or controlled-delivered, helped reduce the significant drop in heart function 1 week after MI. At 2 weeks, although free was still significantly higher than saline (p<0.001), both FAC values kept dropping, while delivery group started diverging and improving function significantly compared to both free and saline (p<0.001). At 5 weeks, free group was still significantly higher than saline (p<0.05), but only at a 36% FAC value compared to 32.5% for saline; whereas, the delivery group increased its improvement of cardiac function compared to both saline and free standing at 47% FAC value (p<0.001). At the terminal 8 weeks, the delivery group stood at 48% FAC value showing significant improvement compared to saline (30%) and free (32%) (p<0.001), while saline and free were statistically similar (p>0.05) (FIG. 21B). The delivery group improved function approximately 74% over saline based on the terminal FAC values at 8 weeks.

The results were confirmed at 8 weeks by cardiac MRI measurements (FIG. 22). ESA and EDA were traced and partial volumes were acquired using slice thickness, then added to compute ESV and EDV values (FIG. 22A). Ejection fraction (EF %) was computed from ESV and EDV relation (FIG. 22B). The delivery group (58%) showed a significantly higher EF % compared to both saline (41%) and free (46%) (p<0.001), which showed no significance between each other (p>0.05). Sham EF % stood at 69%. The delivery group improved function approximately 61% over saline based on the terminal EF values at 8 weeks. This shows that the spatiotemporal delivery of complementary proteins TIMP-3, FGF-2, and SDF-1α can significantly improve cardiac function at least 60% compared to no treatment, suggesting the efficacy of these proteins in mitigating the MI injury and preventing the contractile dysfunction triggered by it.

In our evaluation of the therapy's effect on ventricular dilation, we assessed the changes in ESA and EDA values. ESA values are a more important indicative of the extent of dilation occurring as a result of early ECM degradation and adverse remodeling after MI. As we observed, the saline and free groups showed significantly increasing ESA and EDA values at all time points after MI, with no statistical differences between them (p>0.05) (FIGS. 21C and 21D). The delivery group, on the other hand, showed significant reduction in ESA values at all times after M1 compared to saline (p<0.001), and at 5 and 8 weeks compared to free group (p<0.001) (FIGS. 21C and 21D). The results were confirmed at 8 weeks by cardiac MRI measurements (FIGS. 22C and 22D). The delivery group demonstrated a significantly smaller ESV compared to saline at 8 weeks (p<0.01) (FIG. 22C). These results suggest that the spatiotemporal delivery approach significantly reduces ventricular dilation, and thus in turn reduces the risk of cardiac rupture and heart failure. The ability of the controlled delivery group to improve cardiac function and reduce ventricular dilation up to 8 weeks after infarction, stresses the importance of choosing optimal and complementary proteins and their spatiotemporal presentation per physiologic cues towards achieving high effectiveness in treatment of the infarcted myocardium.

### Spatiotemporal Protein Delivery Augments Myocardial Elasticity

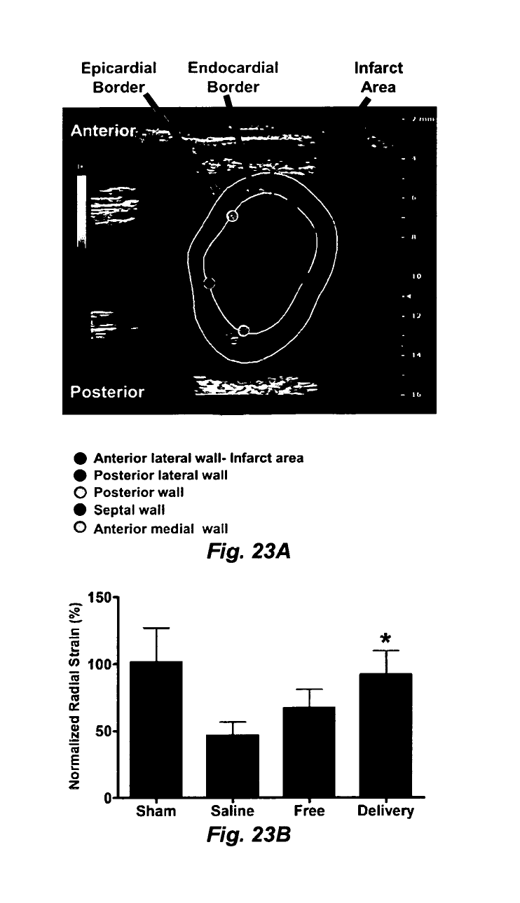

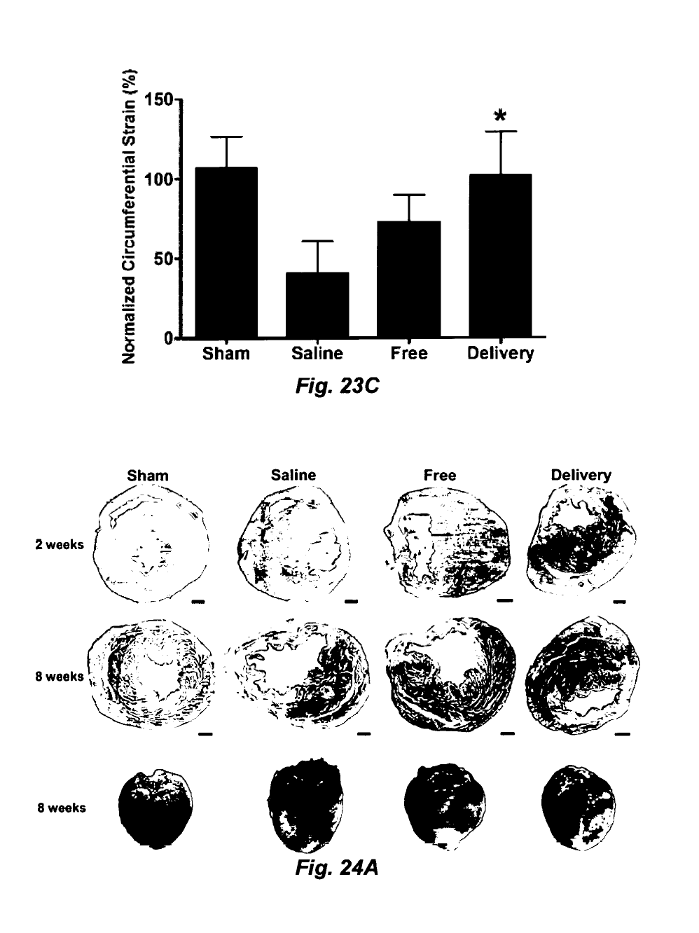

We performed myocardial strain analysis at 8 weeks post-MI to evaluate the changes in the radial and circumferential strain levels of the myocardium after infraction and the effect of therapy on them. The radial strain, defined as the percent change in myocardial wall thickness, and the circumferential strain, defined as the percent change in myocardial circumference, were measured from short-axis view images of the LV from echocardiography using VevoStrain analysis algorithm (FIG. 23A). The strain of an infarcted sample was estimated by normalizing the estimated peak radial or circumferential strain in the infarcted area to that of the average of 4 non-infarct areas in LV walls during a cardiac cycle (FIG. 23A). The free proteins group prevented some reduction in the radial and circumferential strains, but not to a significant level over saline (p>0.05) (FIGS. 23B and 23C). The delivery group, however, was able to significantly maintain the radial (p<0.01) and circumferential (p<0.01) myocardial strains at a higher level in comparison to the saline group keeping the levels very close to those of sham control (FIGS. 23B and 23C). This result indicates the efficacy of the spatiotemporal of complementary proteins TIMP-3, FGF-2, and SDF-1α in preserving the long-term LV myocardial elasticity after MI by preventing the LV wall from becoming stiffer which reduces its ability to contract and dilate properly.

### Spatiotemporal Protein Delivery Reduces LV Wall Thinning and MMP Activity

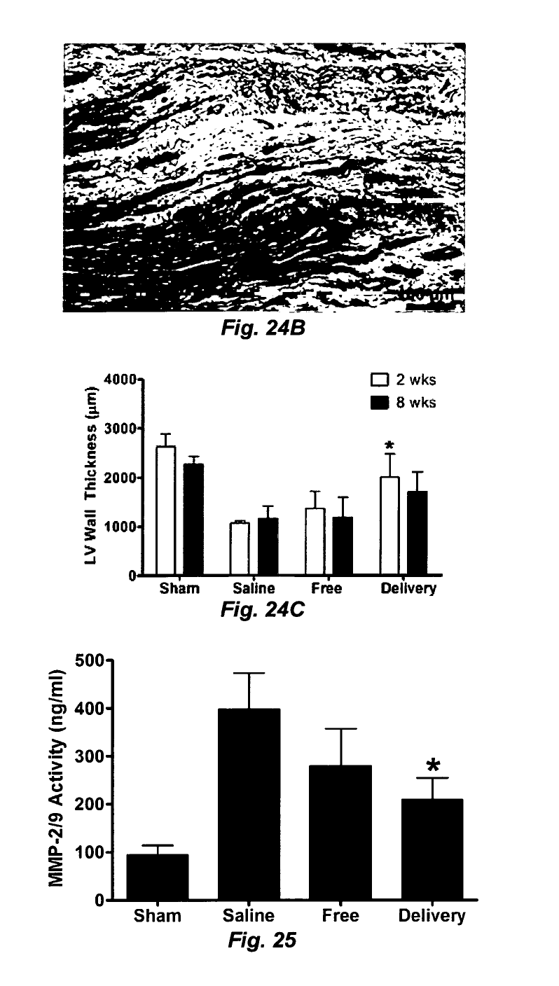

After functional evaluations, we performed investigations at the tissue level at 2 and/or 8 weeks. H&E stained tissue showed increased granulated scar tissue areas with thinner LV walls in the infarct and borderzone regions that exacerbated with time in infarcted groups but to a less extent in the delivery group (FIGS. 24A and 24B). The infarct scar tissue soon starts expanding from the infarct to non-infarct regions turning healthy tissue into collagenous granulated stiffer tissue, clearly evident in non-treated samples (FIG. 24B). LV wall thickness decreased considerably in saline and free groups as early as 2 weeks. In contrast, the delivery group significantly prevented LV wall thinning at 2 weeks compared to saline (FIG. 24C). At 8 weeks, there were no statistical differences in LV wall thickness between saline, free, and delivery groups, although the delivery group clearly maintained a thicker wall average (FIG. 24C).

At 8 weeks, we evaluated the activity of matrix metalloproteinases (MMPs) in the heart samples. MMP-2 and MMP-9 are important players implicated in many cardiovascular diseases and ECM degradation. By allowing activated MMP-2/9 in our samples to cleave a fluorogenic specific substrate, we were able to detect the activity level of MMP-2/9 in the study groups. All infarct groups showed a high level of MMP activity (FIG. 25). However, the delivery group showed significantly lower MMP activity compared to saline (p<0.01) and also lower activity than free group but not to a significant level (p>0.05) (FIG. 25). The enhanced reduction of MMP activity by the delivery group is likely due to the controlled delivery of TIMP-3 within the fibrin gel-coacervate composite, where TIMP-3 can form tight complex with MMP-2 and MMP-9 preventing their activation, and thereby reducing ECM degradation and ventricular dilation and remodeling.

### Spatiotemporal Protein Delivery Reduces Inflammation and Increases M2 Macrophages

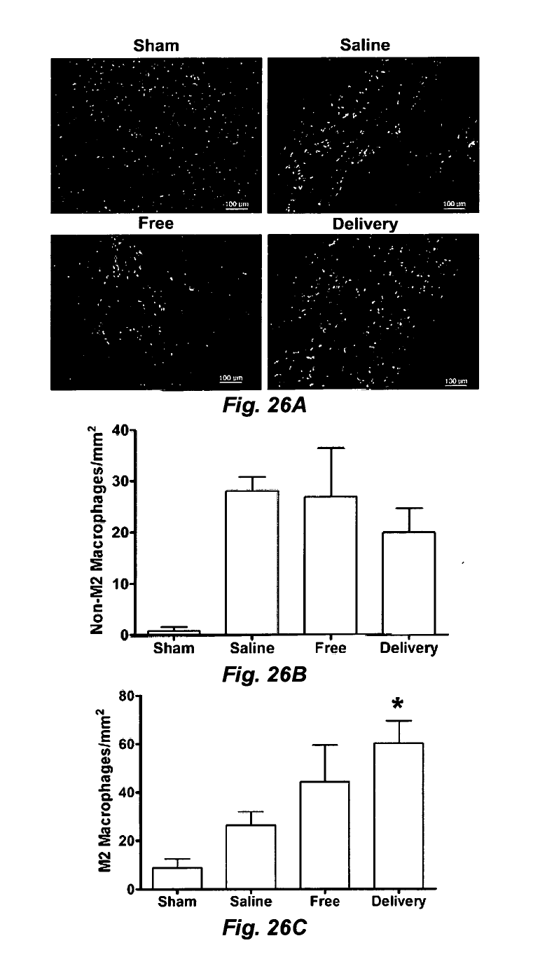

Modulating the inflammatory response after MI in which certain harmful aspects of inflammation are prevented, can be very beneficial for the treatment of the infarcted myocardium. In this study, we assessed inflammation by co-staining for F4/80, a pan-macrophage cell surface marker, and CD163, an M2 macrophage marker (FIG. 26A). Non-M2 macrophages, namely MI, have harmful effects promoting further inflammation, whereas M2 macrophages contribute to tissue repair and anti-inflammation. At 2 weeks post-MI, both saline and free groups showed high numbers of non-M2 macrophages (red in original), while the delivery group showed a trend towards decreasing the presence of such macrophages (p>0.05) (FIGS. 26A and 26B). On the other hand, the delivery group significantly increased the presence of beneficial M2 macrophages (yellow/green in original showing co-staining of F4/80 and CD163) in comparison to saline (p<0.01) (FIGS. 26A and 26C). Saline and free showed no statistical differences in their M2 macrophage numbers (p>0.05) (FIGS. 26A and 26C). These results are suggestive of the efficacy of the spatiotemporal delivery of complementary proteins TIMP-3, FGF-2, and SDF-1α in preventing the infiltration of harmful macrophages into the infracted myocardium or possibly forcing a change in the phenotype of present ones to become of M2 phenotype involved in tissue repair.

### Spatiotemporal Protein Delivery Supports Cardiomyocyte Survival and Reduces Apoptosis

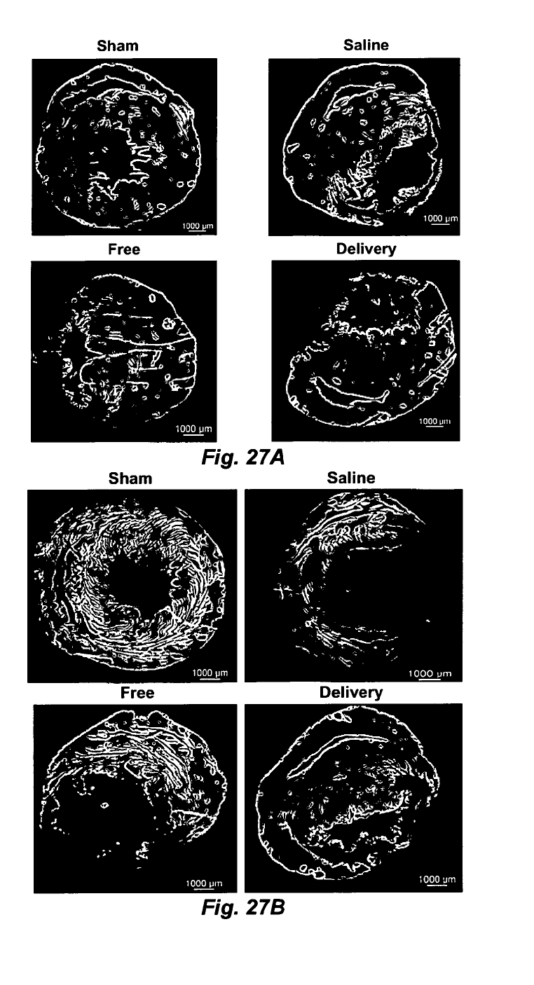

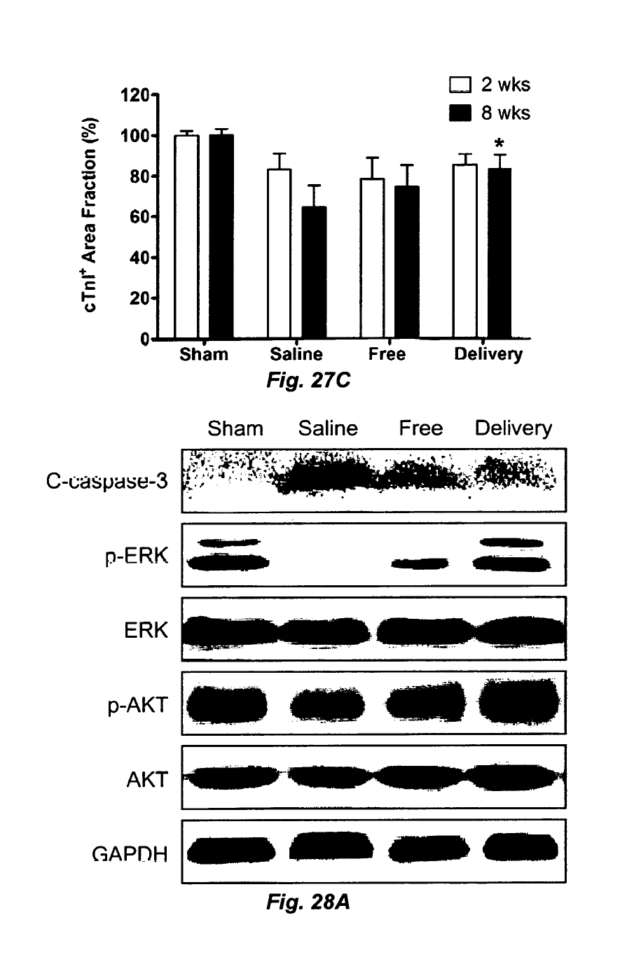

The viability of the cardiac muscle is crucial for the proper function of the heart. Cardiomyocytes are responsible for imparting proper and synchronized contractile ability to the heart for pumping blood. As MI and the pathologies developing after it trigger a massive death of cardiomyocytes, it is extremely beneficial to support the survival of these cells, prevent their apoptosis, and trigger the regeneration of a viable myocardium. To examine the viability of the cardiac muscle, we stained for the live cardiomyocyte marker cardiac troponin I (cTnI) (green) (FIGS. 27A and 12B). We observed a massive amount of non-viable myocardium in the saline followed by the free group, then by the delivery group which apparently preserved the live cardiomyocytes to a larger extent at 2 weeks (FIG. 27A) and at 8 weeks (FIG. 27B). Quantitative analysis of the area fraction of the viable cardiac muscle demonstrated a reduction in the amount of survived cardiomyocytes in all infarct groups at 2 weeks, with no statistical differences between them (p>0.05) (FIG. 27C). At 8 weeks, the viability of the cardiac muscle was reduced more in the saline group (64%), followed by the free group (75%) with no significant differences between them (p>0.05). In contrast, the delivery group was able to maintain the survival of the cardiac muscle (83%) significantly better than saline at 8 weeks (p<0.01) (FIG. 27C).

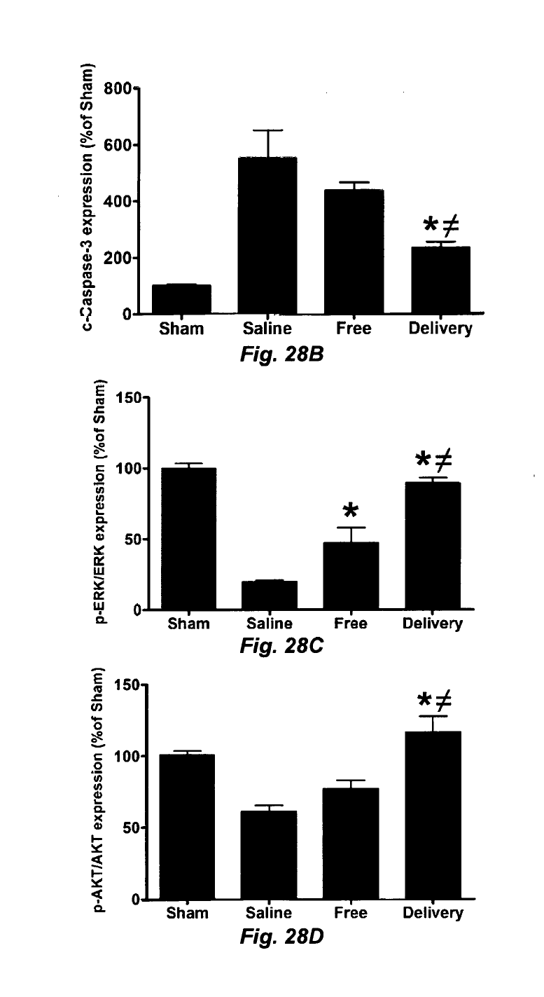

A number of molecular pathways and markers play important roles in inducing survival or apoptosis of the cardiomyocyte. The activated (phosphorylated) MAPK/ERK and Akt pathways have been showed to provide cardioprotective effects supporting the survival of cardiomyocytes after ischemia and preventing their apoptosis. We used western blot to detect the expression levels of cleaved caspase-3, a pro-apoptosis mediator, and pro-survival markers p-ERK1/2 and p-Akt in our groups at 8 weeks (FIG. 28A). As shown in our results, the intensity of the bands is clearly reduced in the delivery group for cleaved caspase-3 and increased in the cases of p-ERK1/2 and p-Akt (FIG. 28A). This was confirmed by quantifying the intensity of the bands (FIG. 28B,C,D). The free group was able to significantly increase p-ERK1/2 expression compared to saline (p<0.01) (FIG. 28B). However, the delivery group significantly reduced the expression of cleaved caspase-3 and increased the expression of p-ERK1/2 and p-Akt compared to both saline (p<0.001) and free (p<0.01) groups (FIG. 28). Taken together, these results demonstrate the effectiveness of the spatiotemporal delivery approach at supporting the long-term survival of cardiomyocytes, preventing their apoptosis, and providing overall cardioprotection after M1 through activation of the Akt and ERK1/2 signaling pathways and the suppression of caspase-3 apoptotic mediation.

### Spatiotemporal Protein Delivery Improves Angiogenesis

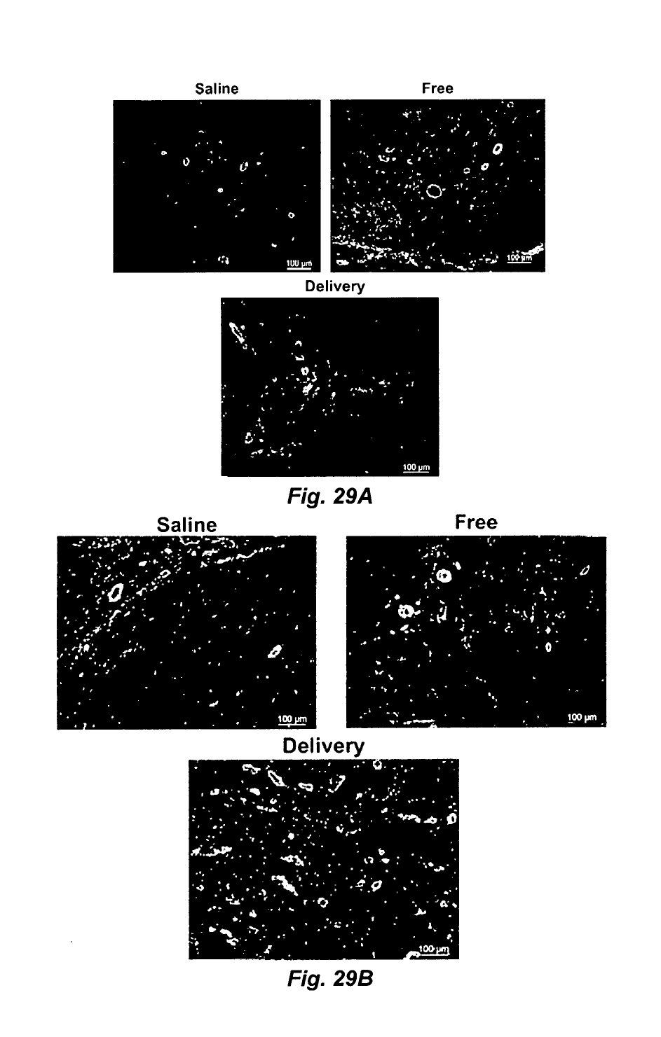

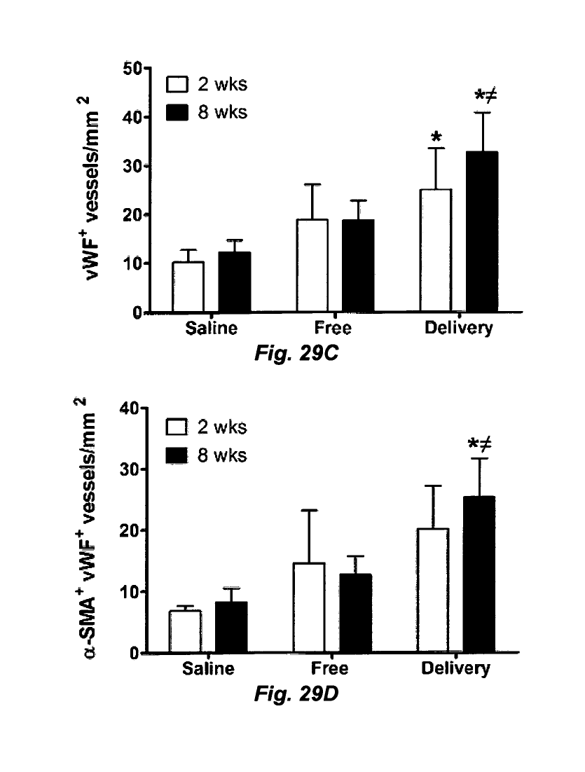

The revascularization of the ischemic myocardium is key to tissue regeneration and functional recovery. New blood vessel formation can help restore the blood, nutrient, and oxygen flow to the damaged myocardial regions, and thereby enhance the survival of cardiomyocytes, reducing the risk of chronic heart failure. To investigate the process of angiogenesis, we co-stained for vWF (red), an endothelial cell marker, and α-SMA (green), a pericyte marker (FIGS. 29A and 29B). Staining for α-SMA indicates higher maturity of a neovessels. At 2 weeks (FIG. 29A) and 8 weeks (FIG. 29B), we observed an increased formation of neovessels in the delivery group compared to saline and free. Quantitative analysis showed significantly higher number of vWF-positive vessels in delivery compared to saline at 2 weeks (p<0.05) (FIG. 29C). At 8 weeks, the delivery group showed a significantly higher number of vWF-positive vessels than both saline and free groups (p<0.01) (FIG. 29C). For the quantitative analysis of the formation of mature neovessels (Co-localized stain of vWF and α-SMA), we found no significant differences in the number of vWF-α-SMA-positive vessels among the infarct groups at 2 weeks (p>0.05) (FIG. 29D). However, at 8 weeks, the delivery group showed significantly higher presence of mature vWF-α-SMA-positive vessels than both saline and free groups (p<0.001) (FIG. 29D). Our results demonstrate the ability of the spatiotemporal delivery approach to induce a robust angiogenesis process capable of forming stable and mature neovasculature that is likely to participate in perfusion. This enhanced revascularization in the delivery group seems likely due to the sustained presence of the potent angiogenic factor FGF-2 being provided by the heparin-based coacervate within our composite gel.

### Spatiotemporal Protein Delivery Increases Stem Cell Homing to the Myocardium

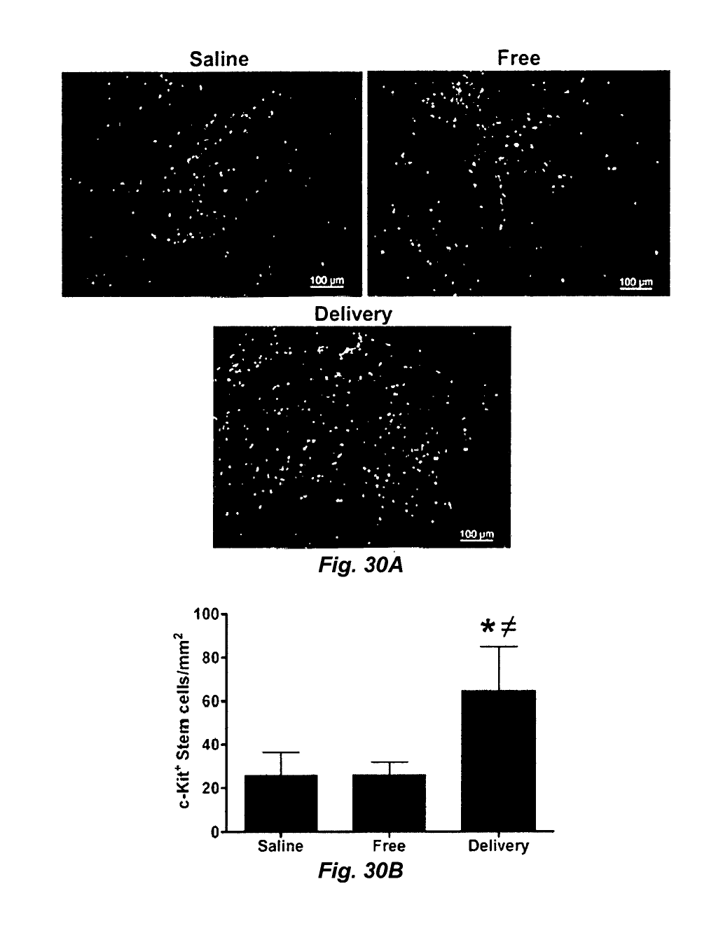

Stem cells, recruited to the infarcted myocardium, have the potential to differentiate into functional cells of cardiac lineages such as cardiomyocytes, vascular endothelial, and mural cells. Stem cells can also impart beneficial paracrine effects that activate repair and regeneration signaling. To examine the homing of stem cells to the infarcted myocardium, we stained for c-Kit, a stem cell marker (FIG. 30A).

At 8 weeks after MI, saline and free groups showed no significant differences in the number of c-Kit-positive cells present at the borderzone (p>0.05) (FIGS. 30A and 30B). In contrast, the delivery group showed a significantly greater presence of c-Kit-positive cells at the borderzone compared to both saline (p<0.01) and free groups (p<0.01) (FIGS. 30A and 30B). This result indicates the efficacy of the spatiotemporal delivery approach in recruiting stem cells to the infarct region and potentially contribute in the regeneration of the myocardium. The enhanced and long-term presence of stem cells in the delivery group is likely due to the sustained availability of the powerful chemoattractant SDF-1α within our composite gel, being released by the coacervate.

Spatiotemporal Protein Delivery Reduces Interstitial Fibrosis after MI

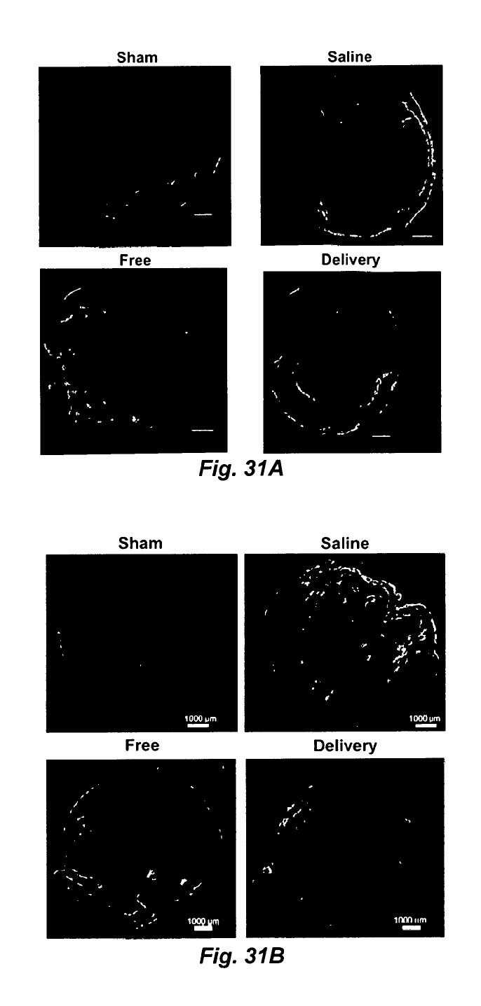

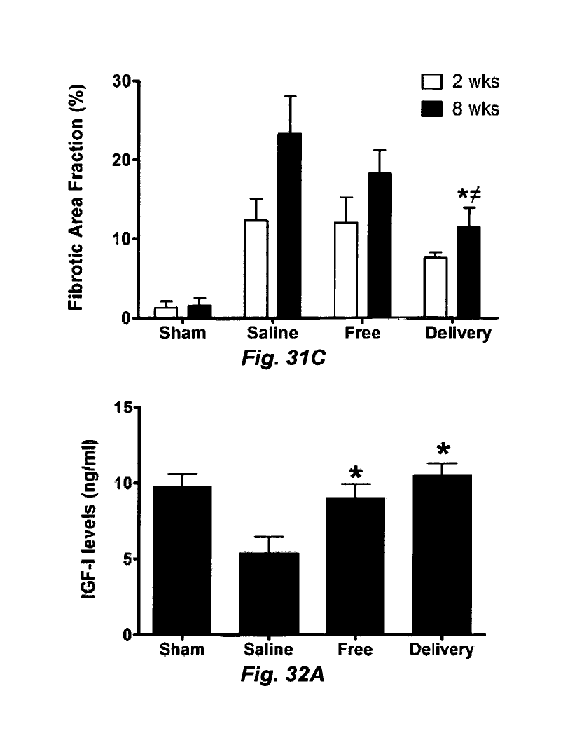

Interstitial fibrosis develops at the infarct region and extends to non-infarct areas due to the excessive and uncontrollable collagen deposition that takes place in later stages after MI. This increased collagen deposition leads to increased stiffness in the myocardium, leading to contractile dysfunction. The extent of fibrosis was assessed using picosirius red staining which stains collagen fibers with a metallic red color than are viewed under polarized light (FIGS. 31A and 31B). The saline group, and to a lesser degree the free group, showed extensive amount of fibrosis that extended from the infarct to non-infract regions, while the delivery group showed far less fibrosis that seemed limited to the infarct area at 2 weeks (FIG. 31A) and at 8 weeks (FIG. 31B). Collagen deposition was quantified as a positive fraction of the heart area and no statistical differences were found between the infarct groups at 2 weeks (p>0.05) (FIG. 31C). At 8 weeks, collagen deposition increased in all infarct groups, but it was found to be significantly less in the delivery group (11%) compared to both saline (23%) (p<0.01) and free (18%) groups (p<0.01) (FIG. 31C).

Spatiotemporal Protein Delivery Regulates Important Protein Signaling after MI

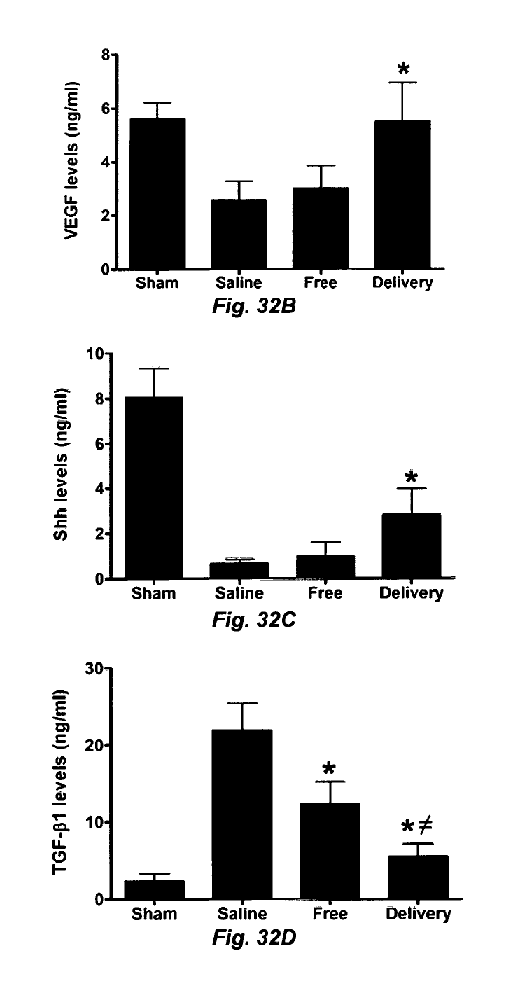

Certain proteins are involved in triggering cardiac repair mechanisms and others are implicated in advancing pathological changes post infarction. Therefore, regulation of the expression levels of such proteins represents an important aspect of effective therapies. The bioavailability and levels of proteins such as the ones in our complementary combination, TIMP-3, FGF-2, and SDF-1α, likely affects the signaling and expression levels of other proteins involved in the heart environment after MI. To investigate the effect of our spatiotemporal delivery approach on the levels of relevant proteins, we tested tissue lysates for the levels of insulin growth factor (IGF-1), vascular endothelial growth factor (VEGF), sonic hedgehog (Shh), and transforming growth factor (TGF-β1) at 8 weeks (FIG. 32). Quantitative analysis by ELISA showed significantly higher levels of IGF-1, an anti-apoptotic factor, in free (p<0.01) and delivery (p<0.001) groups compared to saline (FIG. 32A). Moreover, the delivery group significantly increased the expression levels of VEGF, a potent angiogenic factor, and Shh, a master cardiac morphogen, over saline (p<0.05), while the free group was statistically similar to saline (p>0.05) (FIGS. 32B and 32C). Lastly, the free group significantly decreased the levels of TGF-β1, a pro-fibrotic factor, compared to saline, but the delivery group reduced TGF-β1 levels even more and was significantly less than both saline (p<0.001) and free (p<0.05) groups (FIG. 32D). Taken together, these results suggest a high level of direct and indirect interaction between different proteins during response to tissue injury. The timed and controlled release of our complementary proteins augmented the presence of beneficial factors IGF-1, VEGF, and Shh that likely contributed to increased cardioprotection, revascularization, and signaling of repair mechanisms, while reduced levels of TGF-β1 likely contributed to reducing excessive collagen deposition and interstitial fibrosis. All these beneficial outcomes can aid in the prevention of heart failure and the restoration of heart function.

### CONCLUSIONS

We developed an optimized combination therapy for the repair and regeneration of infarcted myocardium using complementary proteins TIMP-3, FGF-2, and SDF-1α. This therapy is based on a composite of fibrin gel and heparin-based coacervates, where complementary proteins are embedded differently for achieving spatiotemporal release. TIMP-3 was embedded in the fibrin gel achieving early release, while FGF-2 and SDF-1α were encapsulated within heparin-based coacervates and distributed in the same gel achieving sustained release. We found this refined spatiotemporal delivery approach to significantly improve cardiac function up to 8 weeks after MI in rats. In addition, we reported significant improvements in myocardial elasticity, cardiomyocyte survival, angiogenesis, stem cell homing, activation of survival pathways, and important protein signaling. We also reported significant reductions in dilation, ventricular wall thinning, inflammation, MMP activity, fibrosis, and cell apoptosis. Taken together, we believe the more comprehensive a treatment strategy is where multiple crucial proteins are employed and delivered in a spatiotemporal manner, the more this therapy will be successful and effective at cardiac repair. Therefore, the spatiotemporal delivery approach of complementary proteins TIMP-3, FGF-2, and SDF-1α may serve as a new therapy to ameliorate MI injury and set the infarct myocardium on a path to full repair, regeneration, and functional recovery.

Non-limiting aspects or embodiments of the present invention will now be described in the following numbered clauses:

1. A composition comprising: a coacervate of a polycationic polymer, a polyanionic polymer, and a first active agent embedded within a bioerodible, biocompatible hydrogel or a precursor thereof comprising a second active agent. The first and second active agents are the same or different.
2. The composition of clause 1, wherein the polyanionic polymer is a biopolymer.
3. The composition of clause 2, wherein the biopolymer is a heparin or heparan sulfate.
4. The composition of clause 1, wherein the cationic polymer is a polymer composition comprising at least one moiety selected from the following:
(a) [—OC(O)—CH(NHY)—CH₂—C(O)O—CH₂—CH(O—R1)-CH₂—O—CH₂—CH₂—O—CH₂—CH(O—R2)-CH₂—]ₙ,
(b) [—OC(O)—CH₂—CH(NHY)—C(O)O—CH₂—CH(O—R1)-CH₂—O—CH₂—CH₂—O—CH₂—CH(O—R2)-CH₂—]ₙ,
(c) [—OC(O)—CH(NHY)—CH₂—CH₂—C(O)O—CH2-CH(O—R1)-CH₂—O—CH₂—CH₂—O—CH₂—CH(O—R2)-CH₂—]ₙ, and/or
(d) [—OC(O)—CH₂—CH₂—CH(NHY)—C(O)O—CH2-CH(O—R1)-CH₂—O—CH₂—CH₂—O—CH₂—CH(O—R2)-CH₂—]ₙ,
   wherein Y is —C(O)—CH(NH₃⁺)—(CH₂)₃—NH—C(NH₂)₂⁺ or —C(O)—CH(NH₃⁺)—(CH₂)₄—(NH₃)⁺, and R1 and R2 are the same or different and are independently selected from the group consisting of hydrogen, acetylcholine, a carboxy-containing group, an α, β unsaturated carboxylic acid, a cinnamic acid containing group, a p-coumaric acid containing group, a ferulic acid containing group, a caffeic acid containing group, an amine-containing group, a quaternary ammonium containing group, maleic acid, a peptide, maleate, succinate, a phosphate-containing group, and a halo-containing group.
   5. The composition of clause 4, in which cationic polymer is selected from the group consisting of poly(ethylene arginylaspartate diglyceride), poly(ethylene lysinylaspartate diglyceride), poly(ethylene arginylglutamate diglyceride), and poly(ethylene lysinylglutamate diglyceride).
   6. The composition any one of clauses 4-6, the cationic polymer having a polydispersity index of less than 3.0, optionally less than 2.0.
   7. The composition of any one of clauses 4-6, in which one or both of R1 and R2 are a phosphate-containing group, optionally a calcium phosphate selected from the group consisting of hydroxyapatite, apatite, tricalcium phosphate, octacalcium phosphate, calcium hydrogen phosphate, and calcium dihydrogen phosphate.
   8. The composition of any one of clauses 4-6, in which the moiety is complexed with heparin or heparan sulfate.
   9. The composition of any one of clauses 4-6, in which one or both of R1 and R2 are maleate or phosphate.
   10. The composition of any one of clauses 4-6, wherein Y is —C(O)—CH(NH₃⁺)—(CH₂)₄—(NH₃)⁺.
   11. The composition of any one of clauses 4-6, wherein Y is —C(O)—CH(NH₃⁺)—(CH₂)₃—NH—C(NH₂)₂⁺.
   12. The composition of any one of clauses 4-6, in which R1 is hydrogen.
   13. The composition of any one of clauses 4-6, in which one or both of R1 and R2 are charged.
   14. The composition of any one of clauses 1-13, in which the bioerodible, biocompatible hydrogel is a fibrin gel, and/or the bioerodible, biocompatible hydrogel precursor is fibrinogen.
   15. The composition of any one of clauses 1-13, wherein the hydrogel, or a precursor thereof is selected from the group consisting of, fibrin, collagen, gelatin, chitosan, alginate, hyaluronic acid, poly(ethylene glycol), starch, agarose, pectin, silica, PVA, a precursor thereof, and mixtures thereof.
   16. The composition of any one of clauses 1-15, in which the first and/or second active agent comprises a growth factor.
   17. The composition of any one of clauses 1-15, in which the first active agent is PDGF and the second active agent is VEGF.
   18. The composition of any one of clauses 1-15, in which the first and/or second active agent is a biologic, a protein or polypeptide, a growth factor, a chemoattractant, a binding reagent, an antibody or antibody fragment, a receptor or a receptor fragment, a ligand, or an antigen and/or an epitope.
   19. The composition of any one of clauses 1-18, in which the first and second active agents are independently selected from the group consisting of: VEGF, HGF, PDGF, TIMP-3, FGF-2, SDF-1α, IL-10, Ang1, Ang2, IGF-1, relaxin, Shh, FGF-1, NRG-1, and BMP-2.
   20. The composition of clause 1, in which the polycationic polymer is PEAD, the polyanionic polymer is heparin, the first active agent is PDGF, the bioerodible, biocompatible hydrogel or a precursor thereof is fibrin or fibrinogen, and the second active agent is VEGF.
   21. A composition comprising: a coacervate of a polycationic polymer, a polyanionic polymer, FGF-2, and SDF-1α embedded within a bioerodible, biocompatible hydrogel comprising TIMP3 or a precursor thereof.
   22. The composition of clause 21, wherein the hydrogel or a precursor thereof is fibrin or fibrinogen.
   23. The composition of clause 22, wherein the hydrogel, or a precursor thereof is selected from the group consisting of, fibrin, collagen, gelatin, chitosan, alginate, hyaluronic acid, polyethyleneglycol, starch, agarose, pectin, silica, PVA, a precursor thereof, and mixtures thereof.
   24. The composition of any of clauses 21-23, wherein the polyanionic polymer is heparin or heparan sulfate, the polycationic polymer is a polymer composition comprising at least one moiety selected from the following:
(a) [—OC(O)—CH(NHY)—CH₂—C(O)O—CH₂—CH(O—R1)-CH₂—O—CH₂—CH₂—O—CH₂—CH(O—R2)-CH₂—]ₙ,
(b) [—OC(O)—CH₂—CH(NHY)—C(O)O—CH₂—CH(O—R1)-CH₂—O—CH₂—CH₂—O—CH₂—CH(O—R2)-CH₂—]ₙ,
(c) [—OC(O)—CH(NHY)—CH₂—CH₂—C(O)O—CH2-CH(O—R1)-CH₂—O—CH₂—CH₂—O—CH₂—CH(O—R2)-CH₂—]ₙ, and/or
(d) [—OC(O)—CH₂—CH₂—CH(NHY)—C(O)O—CH2-CH(O—R1)-CH₂—O—CH₂—CH₂—O—CH₂—CH(O—R2)-CH₂—]ₙ,
   wherein Y is —C(O)—CH(NH₃⁺)—(CH₂)₃—NH—C(NH₂)₂⁺ or —C(O)—CH(NH₃⁺)—(CH₂)₄—(NH₃)⁺, and R1 and R2 are the same or different and are independently selected from the group consisting of hydrogen, acetylcholine, a carboxy-containing group, an α, β unsaturated carboxylic acid, a cinnamic acid containing group, a p-coumaric acid containing group, a ferulic acid containing group, a caffeic acid containing group, an amine-containing group, a quaternary ammonium containing group, maleic acid, a peptide, maleate, succinate, a phosphate-containing group, and a halo-containing group.
   25. The composition of any one of clauses 21-24, wherein the polycationic polymer is selected from the group consisting of poly(ethylene arginylaspartate diglyceride), poly(ethylene lysinylaspartate diglyceride), poly(ethylene arginylglutamate diglyceride), and poly(ethylene lysinylglutamate diglyceride).
   26. The composition of any one of clauses 21-25, further comprising IL-10 (Interleukin 10), optionally embedded in the hydrogel.
   27. The composition of any one of clauses 1-26, in which the zeta potential of the aggregated coacervate ranges from −15 mV to 15 mv, −10 mV to 10 mV, or −5 mV to 5 mV.
   28. A method of treating a myocardial infarct in which the composition of any one of clauses 21-27 is delivered, for example, injected, at or immediately adjacent to the site of the infarct.
   29. A method of delivering a plurality of active agents to a patient in need thereof, in a spatiotemporal pattern, comprising injecting or otherwise introducing the composition of any of clauses 1-27 into the patient.
   30. A method of treating ischemia and/or promoting bone generation or regeneration or tissue growth, comprising delivering into a site on a patient the composition of any of clauses 1-27.
   31. The method of clause 30, for treating a myocardial infarct, where the composition is delivered, for example, injected, at or immediately adjacent to the site of the infarct.
   32. A method of making a drug delivery composition, comprising:
a. mixing a polycationic polymer, a polyanionic polymer, and one or more first active agents in amounts effective to produce a coacervate;
b. mixing the coacervate into a bioerodible, biocompatible hydrogel or a precursor thereof and one or more second active agents, wherein the one or more first active agents is/are the same or different from the one or more second active agents.
   33. The method of clause 32, wherein the polyanionic polymer is a biopolymer.
   34. The method of clause 33, wherein the biopolymer is a heparin or heparan sulfate.
   35. The method of clause 32, wherein the cationic polymer is a polymer composition comprising at least one moiety selected from the following:
(a) [—OC(O)—CH(NHY)—CH₂—C(O)O—CH₂—CH(O—R1)-CH₂—O—CH₂—CH₂—O—CH₂—CH(O—R2)-CH₂—]ₙ,
(b) [—OC(O)—CH₂—CH(NHY)—C(O)O—CH₂—CH(O—R1)-CH₂—O—CH₂—CH₂—O—CH₂—CH(O—R2)-CH₂—]ₙ,
(c) [—OC(O)—CH(NHY)—CH₂—CH₂—C(O)O—CH2-CH(O—R1)-CH₂—O—CH₂—CH₂—O—CH₂—CH(O—R2)-CH₂—]ₙ, and/or
(d) [—OC(O)—CH₂—CH₂—CH(NHY)—C(O)O—CH2-CH(O—R1)-CH₂—O—CH2-CH₂—O—CH₂—CH(O—R2)-CH₂-]ₙ,
   wherein Y is —C(O)—CH(NH₃⁺)—(CH₂)₃—NH—C(NH₂)₂⁺ or —C(O)—CH(NH₃⁺)—(CH₂)₄—(NH₃)⁺, and R1 and R2 are the same or different and are independently selected from the group consisting of hydrogen, acetylcholine, a carboxy-containing group, an α, β unsaturated carboxylic acid, a cinnamic acid containing group, a p-coumaric acid containing group, a ferulic acid containing group, a caffeic acid containing group, an amine-containing group, a quaternary ammonium containing group, maleic acid, a peptide, maleate, succinate, a phosphate-containing group, and a halo-containing group.
   36. The method of clause 35, in which cationic polymer is selected from the group consisting of poly(ethylene arginylaspartate diglyceride), poly(ethylene lysinylaspartate diglyceride), poly(ethylene arginylglutamate diglyceride), and poly(ethylene lysinylglutamate diglyceride).
   37. The method of clause 35 or 36, the cationic polymer having a polydispersity index of less than 3.0, optionally less than 2.0.
   38. The method of any one of clauses 35-37, in which one or both of R1 and R2 are a phosphate-containing group, optionally a calcium phosphate selected from the group consisting of hydroxyapatite, apatite, tricalcium phosphate, octacalcium phosphate, calcium hydrogen phosphate, and calcium dihydrogen phosphate.
   39. The method of any one of clauses 35-37, in which the moiety is complexed with heparin or heparan sulfate.
   40. The method of any one of clauses 35-37, in which one or both of R1 and R2 are maleate or phosphate.
   41. The method of any one of clauses 35-37, wherein Y is —C(O)—CH(NH₃⁺)—(CH₂)₄—(NH₃)⁺.
   42. The method of any one of clauses 35-37, wherein Y is —C(O)—CH(NH₃⁺)—(CH₂)₃—NH—C(NH₂)₂⁺.
   43. The method of any one of clauses 35-37, in which R1 is hydrogen.
   44. The method of any one of clauses 35-37, in which one or both of R1 and R2 are charged.
   45. The method of any one of clauses 32-44, in which the bioerodible, biocompatible hydrogel is a fibrin gel, and/or the bioerodible, biocompatible hydrogel precursor is fibrinogen.
   46. The method of any one of clauses 32-44, wherein the hydrogel, or a precursor thereof is selected from the group consisting of, fibrin, collagen, gelatin, chitosan, alginate, hyaluronic acid, poly(ethylene glycol), starch, agarose, pectin, silica, PVA, a precursor thereof, and mixtures thereof.
   47. The method of any one of clauses 32-44, in which one or more of the first and/or second active agents is a growth factor.
   48. The method of any one of clauses 32-44, in which the one or more first active agents is PDGF and the one or more second active agents is VEGF.
   49. The method of any one of clauses 32-44, in which one or more of the one or more first and/or second active agents is a biologic, a protein or polypeptide, a growth factor, a chemoattractant, a binding reagent, an antibody or antibody fragment, a receptor or a receptor fragment, a ligand, or an antigen and/or an epitope.
   50. The method of any one of clauses 32-44, in which the one or more first and second active agents are independently selected from the group consisting of: VEGF, HGF, PDGF, TIMP-3, FGF-2, SDF-1α, IL-10, Ang1, Ang2, IGF-1, relaxin, Shh, FGF-1, NRG-1, and BMP-2.
   51. The method of clause 32, in which the polycationic polymer is PEAD, the polyanionic polymer is heparin, the one or more first active agents is PDGF, the bioerodible, biocompatible hydrogel or a precursor thereof is fibrin or fibrinogen, and the one or more second active agents is VEGF.
   52. The method of any one of clauses 32-46, wherein the one or more first active agents are FGF-2 and SDF-1α, and the one or more second active agents TIMP3.
   53. The method of any one of clauses 32-52, in which the zeta potential of the aggregated coacervate ranges from −15 mV to 15 mv, −10 mV to 10 mV, or −5 mV to 5 mV.

1. A composition comprising: a coacervate of a polycationic polymer, a polyanionic polymer, and a first active agent embedded within a bioerodible, biocompatible hydrogel or a precursor thereof comprising a second active agent.

2. The composition of claim 1, wherein the polyanionic polymer is a heparin or heparan sulfate.

3. The composition of claim 1, wherein the cationic polymer is a polymer composition comprising at least one moiety selected from the following:
(a) [—OC(O)—CH(NHY)—CH₂—C(O)O—CH₂—CH(O—R1)-CH₂—O—CH₂—CH₂—O—CH₂—CH(O—R2)-CH₂—]ₙ,
(b) [—OC(O)—CH₂—CH(NHY)—C(O)O—CH₂—CH(O—R1)-CH₂—O—CH₂—CH₂—O—CH₂—CH(O—R2)-CH₂—]ₙ,
(c) [—OC(O)—CH(NHY)—CH₂—CH₂—C(O)O—CH2-CH(O—R1)-CH₂—O—CH₂—CH₂—O—CH₂—CH(O—R2)-CH₂—]ₙ, and/or
(d) [—OC(O)—CH₂—CH₂—CH(NHY)—C(O)O—CH2-CH(O—R1)-CH₂—O—CH₂—CH₂—O—CH₂—CH(O—R2)-CH₂—]ₙ,
wherein Y is —C(O)—CH(NH₃⁺)—(CH₂)₃—NH—C(NH₂)₂⁺ or —C(O)—CH(NH₃⁺)—(CH₂)₄—(NH₃)⁺, and R1 and R2 are the same or different and are independently selected from the group consisting of hydrogen, acetylcholine, a carboxy-containing group, an α, β unsaturated carboxylic acid, a cinnamic acid containing group, a p-coumaric acid containing group, a ferulic acid containing group, a caffeic acid containing group, an amine-containing group, a quaternary ammonium containing group, maleic acid, a peptide, maleate, succinate, a phosphate-containing group, and a halo-containing group.

4. The composition of claim 1, in which cationic polymer is selected from the group consisting of poly(ethylene arginylaspartate diglyceride), poly(ethylene lysinylaspartate diglyceride), poly(ethylene arginylglutamate diglyceride), and poly(ethylene lysinylglutamate diglyceride).

5. (canceled)

6. The composition of any one of claim 1, wherein the hydrogel, or a precursor thereof is selected from the group consisting of, fibrin, collagen, gelatin, chitosan, alginate, hyaluronic acid, poly(ethylene glycol), starch, agarose, pectin, silica, PVA, a precursor thereof, fibrinogen, and mixtures thereof.

7. The composition of claim 1, in which the first and/or second active agents include one or more growth factors.

8. The composition of claim 1, wherein the first and second active agents are independently selected from the group consisting of: VEGF, HGF, PDGF, TIMP-3, FGF-2, SDF-1α, IL-10, Ang1, Ang2, IGF-1, relaxin, Shh, FGF-1, NRG-1, and BMP-2.

9. The composition of claim 1, in which the polycationic polymer is PEAD, the polyanionic polymer is heparin, the first active agent is PDGF, the bioerodible, biocompatible hydrogel or a precursor thereof is fibrin or fibrinogen, and the second active agent is VEGF.

10. The composition of claim 1, wherein the first active agent comprises FGF-2 and SDF-1α, and the second active agent is TIMP3.

11. The composition of claim 1, wherein the zeta potential of the aggregated coacervate ranges from −15 mV to 15 mv.

12. (canceled)

13. A method of delivering a plurality of active agents to a patient in need thereof, in a spatiotemporal pattern, comprising introducing the composition of claim 1 into the patient.

14. A method of treating ischemia and/or promoting bone generation or regeneration or tissue growth, comprising delivering into a site on a patient the composition of claim 1.

15. The method of claim 14, for treating a myocardial infarct, where the composition is delivered, at or immediately adjacent to the site of the infarct.

16. A method of making a drug delivery composition, comprising:
a. mixing a polycationic polymer, a polyanionic polymer, and one or more first active agents in amounts effective to produce a coacervate;
b. mixing the coacervate into a bioerodible, biocompatible hydrogel or a precursor thereof and one or more second active agents.

17. The method of claim 16, wherein the polyanionic polymer is a heparin or heparan sulfate.

18. The method of claim 16, wherein the cationic polymer is a polymer composition comprising at least one moiety selected from the following:
(a) [—OC(O)—CH(NHY)—CH₂—C(O)O—CH₂—CH(O—R1)-CH₂—O—CH₂—CH₂—O—CH₂—CH(O—R2)-CH₂—]ₙ,
(b) [—OC(O)—CH₂—CH(NHY)—C(O)O—CH₂—CH(O—R1)-CH₂—O—CH₂—CH₂—O—CH₂—CH(O—R2)-CH₂—]ₙ,
(c) [—OC(O)—CH(NHY)—CH₂—CH₂—C(O)O—CH2-CH(O—R1)-CH₂—O—CH₂—CH₂—O—CH₂—CH(O—R2)-CH₂—]ₙ, and/or
(d) [—OC(O)—CH₂—CH₂—CH(NHY)—C(O)O—CH2-CH(O—R1)-CH₂—O—CH₂—CH₂—O—CH₂—CH(O—R2)-CH₂—]ₙ,
wherein Y is —C(O)—CH(NH₃⁺)—(CH₂)₃—NH—C(NH₂)₂⁺ or —C(O)—CH(NH₃⁺)—(CH₂)₄—(NH₃)⁺, and R1 and R2 are the same or different and are independently selected from the group consisting of hydrogen, acetylcholine, a carboxy-containing group, an α, β unsaturated carboxylic acid, a cinnamic acid containing group, a p-coumaric acid containing group, a ferulic acid containing group, a caffeic acid containing group, an amine-containing group, a quaternary ammonium containing group, maleic acid, a peptide, maleate, succinate, a phosphate-containing group, and a halo-containing group.

19. The method of claim 16, in which cationic polymer is selected from the group consisting of poly(ethylene arginylaspartate diglyceride), poly(ethylene lysinylaspartate diglyceride), poly(ethylene arginylglutamate diglyceride), and poly(ethylene lysinylglutamate diglyceride).

20. (canceled)

21. The method of claim 16, wherein the hydrogel, or a precursor thereof is selected from the group consisting of, fibrin, collagen, gelatin, chitosan, alginate, hyaluronic acid, poly(ethylene glycol), starch, agarose, pectin, silica, PVA, a precursor thereof, fibrinogen, and mixtures thereof.

22-26. (canceled)

27. The method of claim 14, wherein the polycationic polymer is PEAD, the polyanionic polymer is heparin, the first active agent is PDGF, the bioerodible, biocompatible hydrogel or a precursor thereof is fibrin or fibrinogen, and the second active agent is VEGF.

28. The method of claim 14, wherein the first active agent comprises FGF-2 and SDF-1α, and the second active agent is TIMP3.


SEQUENCE LISTING (19 sequences)

SEQ ID NO: 1 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 2 - protein, 154 aa, Homo sapiens
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 4 - protein, 109 aa, Homo sapiens
SLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT

SEQ ID NO: 5 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 6 - DNA, 5496 bp, Homo sapiens
ctgtggcttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctgcaaacagcagatggcttcccatatcccagagagtaagaaccagagagagagagaaagagagagagtttgggtctttctcctctgtgcctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggcgcgccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagcggcgcagagcgggcagcaggcaggcggcgggcgctcagacggcttctcctcctcctcttgctcctccagctcctgctccttcgccgggaggccgcccgccgagtcctgcgccagcgccgaggcagcctcgctgcgccccatcccgtcccgccgggcactcggagggcagcgcgccggaggccaaggttgccccgcacggcccggcgggcgagcgagctcgggctgcagcagccccgccggcggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccatacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaacaaaaatgaaaaactactccatttgaggattgtaattcccacccctcttgcttcttccccacctcaccatctcccagaccctcttccctttgcccttctcctccaatacataaaggacacagacaaggaacttgctgaaaggccaaccatttcaggatcagtcaaaggcagcaagcagatagactcaaggtgtgtgaaagatgttatacaccaggagctgccactgcatgtcccaaccagactgtgtctgtctgtgtctgcatgtaagagtgagggagggaaggaaggaactacaagagagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatggggacataagctaatttttttacaggacacagaattctgttcaatgctgttaaatatgccaatagtttaatctcttctattttgttgtcgttgcttgtttgaagaaaatcatgacattccaagttgacatttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttgtagggtttctgttgtgtttttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcagttcattccactttaggaaacagagctgccaattgaaacagaagaagaaaaaaaaaaaaagcagcagacaacacactgtagagtcttgcacacacacaagtgcccaggcaaggtgcttggcagaaccgcagagtgggaagagagtaccggcatcgggtttccttgggatcaatttcattaccgtgtacctttcccattgtggtcatgccatttggcagggggagaatgggaggcttggccttctttgtgaggcagtgtgagcagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcagaaccacatccccatctgttaggccttggtgaggaggccctgggcaaagaagggtctttcgcaaagcgatgtcagagggcggttttgagctttctataagctatagctttgtttatttcacccgttcacttactgtataatttaaaatcatttatgtagctgagacacttctgtatttcaatcatatcatgaacattttattttgctaaatcttgtgtcatgtgtaggctgtaatatgtgtacattgtgtttaagagaaaaatgaaacccacatgccgccattttcctgaatcaaattctgcagtggaatggagaggaaaatacttctaggcaagcagctagactggtgaattgggggaaatagaaggaactagtaactgagactcctccagcctcctccctattggaatcccaatggctcctggagtaggaaaaaagtttaaactacattcatgttcttgttctgtgtcactcggccctgggtagtctaccatttacttcaccccaagtcctgctgcccatccagttgggaagccatgattttcctaagaatccagggccatgggagatacaattccaagttctcgcttcctcctttgggcatctcttctgcctcccaatcaaggaagctccatgctcaggctctcagctctcgggccagtgctctgctctgtccagggtaggtaatactgggagactcctgtcttttaccctcccctcgttccagacctgcctcatggtggcaacatggttcttgaacaattaaagaaacaaatgactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttcactttttatagtgtgtcctttattctaaacagtaaagtggttttatttctatcacaaaaaaaaaaa

SEQ ID NO: 7 - DNA, 1940 bp, Homo sapiens
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggtataaacgtcctactgtatgtgggaatttatttacctgaaataaaattcattagttgttagtgatggagcttaaaaaaaa

SEQ ID NO: 8 - protein, 5 aa, Homo sapiens
IKVAV

SEQ ID NO: 9 - protein, 4 aa, Homo sapiens
RGDS

SEQ ID NO: 10 - protein, 6 aa, Homo sapiens
KQAGDV

SEQ ID NO: 11 - protein, 6 aa, Homo sapiens
VAPGVG

SEQ ID NO: 12 - protein, 6 aa, Homo sapiens
APGVGV

SEQ ID NO: 13 - protein, 6 aa, Homo sapiens
PGVGVA

SEQ ID NO: 14 - protein, 5 aa, Homo sapiens
GVGVA

SEQ ID NO: 15 - protein, 4 aa, Homo sapiens
VAPG

SEQ ID NO: 16 - protein, 6 aa, Homo sapiens
VGVAPG

SEQ ID NO: 17 - protein, 4 aa, Homo sapiens
VGVA

SEQ ID NO: 18 - protein, 5 aa, Homo sapiens
VAPGV

SEQ ID NO: 19 - protein, 6 aa, Homo sapiens
GVAPGV